(19) United States
(12) Patent Application Publication
(10) Pub. No.: US 2008/0152640 A1
(43) Pub. Date: Jun. 26, 2008

(54) Means and Methods For Treating a Disease Which is Associated With an Excess Transport of Hyaluronan Across a Lipid Bilayer

(76) Inventor: Peter Prehm, Nottuln (DE)

Correspondence Address: PERKINS COIE LLP, P.O. BOX 2168, MENLO PARK, CA, 94026, US

(21) Appl. No.: 10/566,145

(22) PCT Filed: Jul. 29, 2004
(86) PCT No.: PCT/EP04/08547

§ 371 Date: Dec. 20, 2007

(30) Foreign Application Priority Data
Jul. 29, 2003 (EP) 03016615.1
Jul. 31, 2003 (EP) 03017374.4
Oct. 31, 2003 (EP) 03025102.9
May 25, 2004 (EP) 04012369.7

(51) Int. Cl. A61K 31/4422; A61K 31/135; A61K 38/13; A61K 31/473; A61K 31/454; A61K 31/4965; A61K 31/405; A61K 31/519; A61P 19/02; G01N 33/53; A61K 31/70; A61K 31/155; A61K 31/19; A61K 31/40; A61K 31/495; A61K 31/439; A61K 39/395

(52) U.S. Cl. 424/130.1; 514/654; 514/9; 514/297; 514/305; 514/356; 514/255.03; 514/322; 514/428; 514/353; 514/562; 514/420; 514/635; 514/249; 514/44; 435/7.92; 435/7.1

(57) ABSTRACT

The present invention relates to the use of at least one inhibitor of at least one ABC-transporter capable of transporting hyaluronan across a lipid bilayer, such as verapamil or valspodar, for the preparation of a pharmaceutical composition for the treatment of a disease which is associated with an excess transport of hyaluronan across a lipid bilayer, e.g. arthritis. Furthermore, the present invention relates to a method for screening a compound which is suitable for the treatment of a disease which is associated with an excess transport of hyaluronan across a lipid bilayer, e.g. arthritis. The present invention also relates to a method for screening a compound which reduces the transport of hyaluronan mediated by (an) ABC-transporter(s). Furthermore, the present invention relates to a method for identifying a subject at risk for a disease which is associated with an excess transport of hyaluronan across a lipid bilayer, e.g. arthritis as well as to a method of screening for a compound which is suitable for the treatment of a disease which is associated with an excess transport of hyaluronan across a lipid bilayer, e.g. arthritis in a subject. In addition, the present invention relates to a method of preventing, ameliorating and/or treating the symptoms of a disease which is associated with an excess transport of hyaluronan across a lipid bilayer, e.g. arthritis in a subject.

The present invention relates to the use of at least one inhibitor of at least one ABC-transporter capable of transporting hyaluronan across a lipid bilayer, for the preparation of a pharmaceutical composition for the treatment of a disease which is associated with an excess transport of hyaluronan across a lipid bilayer, e.g. arthritis. Furthermore, the present invention relates to a method for screening a compound which is suitable for the treatment of a disease which is associated with an excess transport of hyaluronan across a lipid bilayer, e.g. arthritis. The present invention also relates to a method for screening a compound which reduces the transport of hyaluronan mediated by (an) ABC-transporter(s). Furthermore, the present invention relates to a method for identifying a subject at risk for a disease which is associated with an excess transport of hyaluronan across a lipid bilayer, e.g. arthritis as well as to a method of screening for a compound which is suitable for the treatment of a disease which is associated with an excess transport of hyaluronan across a lipid bilayer, e.g. arthritis in a subject. In addition, the present invention relates to a method of preventing, ameliorating and/or treating the symptoms of a disease which is associated with an excess transport of hyaluronan across a lipid bilayer, e.g. arthritis in a subject.

A variety of documents is cited throughout this specification. The disclosure content of said documents (including any manufacturer's specifications, instructions etc.) is herewith incorporated by reference; however, there is no admission that any document cited is indeed prior art as to the present invention.

Arthritis is a very common disease: its prevalence is increasing with age and is 9% at an age of 20 years, 17% at 35 and 90% over 65. Symptoms are common at an age between 50 and 60. The pathogenesis is favoured by several risk factors: genetical disposition, joint injury, obesity, joint deformity, local biomechanical factors and inflammation. The total annual costs for medial treatment and economical loss caused by disability has been calculated for Germany to amount to 12 billion [symbol] . No other disease has a larger pharmaceutical market.

Arthritis is accompanied with a loss of cartilage at the joint surface. The cartilage goes through different stages during pathogenesis. At first chondrocytes try to replace loss of cartilage by increased synthesis and proliferation; simultaneously lacunae of edema and increased water binding occurs. Increased water binding leads to softening of the cartilage matrix. The cause of edema and water accumulation is known form other systems: an increased hyaluronan production. These phenomena are observed before fibrillation or cartilage erosion. At the second stage new cartilage production cannot compensate for the loss and at the third stage loss of cartilage is complete.

The cartilage matrix consists of two main components: type II collagen and high molecular weight aggrecan. Aggrecan is a proteoglycan and is composed of a core-protein that binds many molecules of chondroitin sulfate and keratan sulfate. These proteoglycans decorate a backbone of hyaluronan like a bristles of a bottle brush. Hyaluronan itself is anchored in the membrane of chondrocytes at the membrane integrated synthase. It is further bound by the cell surface receptor CD44.

It is known that the CD44 receptor transmits signals into the cell upon binding to oligosaccharides of hyaluronan and causes increased production of metalloproteases [19]. Therefore it appears likely that an enhanced hyaluronan concentration in osteoarthritic cartilage has a similar effect and also induces protease production.

In spite of this knowledge, academic and industrial research spends enormous efforts for the development of protease inhibitors for treatment of arthritis. Since the discovery of collagenase 1962 almost every large pharmaceutical company has had a protease inhibitor program. Although there were some partial benefits in animal models [20], there is still not a single approved drug and the therapeutic success of protease inhibitors were very sobering [21]. This is not surprising, because protease inhibitors do not inhibit the primary process.

The consequence of joint injury has some similarity with the swelling of a lump after contusion. Also here hyaluronan production is the primary process accompanied by water accumulation that precedes the activation of proteases and other inflammatory reactions. The swollen tissue enables the invasion of leukocytes and inflammation.

Up to now no therapy exists that can alter the course of the disease or can repair existing damages. Treatment is confined to pain relieve by physiotherapy, analgetic or anti-inflammatory drugs or intraarticularly applied hyaluronan. Intra-articular administration of hyaluronan has been used in animals and man. In man, hyaluronan is being used to relieve pain and improve joint mobility in the treatment of arthritis with intra-articular injections of Hyalgan® (Sanofi Pharmaceuticals), Orthovisc® (Anika Therapeutics), and SynVisc® (Biomatrix, now Genzyme). It has also been proposed for several degenerative joint diseases as an alternative to the traditional steroid therapy [32-34]. However, the benefit of hyaluronan injections remains controversial [1].

Most injuries are followed by inflammation and hyaluronan overproduction that may lead to severe health problems. Excess hyaluronan production is observed after organ transplantation that may lead to tissue rejection, and after a heart infarct. It is also associated with alveolitis, pancreatitis, pulmonary or hepatic fibrosis, radiation induced inflammation, Crohn's disease, myocarditis, scleroderma, psoriasis, sarcoidosis [119-135]. Also many human tumors are characterized by an overproduction of hyaluronan such as melanoma [89], mesothelioma [117] or colon carcinoma [118]. Because hyaluronan production is correlated with cell proliferation [86], inhibition of hyaluronan transport will also reduce tumour growth. Overproduction of hyaluronan is also the cause of lump formation after contusion or insect bites, therefore it will be possible to inhibit swelling by the inhibitors of hyaluronan transport.

Increased hyaluronan production and accumulation in the interstitium can cause dangerous edema that can impair organ function. Thus increased hyaluronan production around blood vessels will compress the vessels and cause increased blood pressure and decreased oxygen supply. This may lead to irreversible damage, tissue rejection and life threatening situations. There is an urgent requirement for drugs that suppress hyaluronan synthesis. Although there are many patented procedures and agents that inhibit edema formation, there is no drug that is directed against the cause of edema formation: increased hyaluronan production. Edema were reduced by treatment with hyaluronidases (WO9808538; WO9603497), but this procedure is far from optimal, because the hyaluronidase can cause allergic and immune reactions.

Thus, the technical problem underlying the present invention was to provide means and methods for treating and/or preventing a disease which is associated with an excess transport of hyaluronan across a lipid bilayer, e.g. arthritis.

The solution to said technical problem is achieved by providing the embodiments characterized in the claims.

Hyaluronan is present in all vertebrates and also in the capsule of some pathogenic bacteria such as Streptococci and Pasteurella. It is a component of extracellular matrices in most tissues and in some tissues it is a major constituent. The concentration of hyaluronan is particularly high in rooster comb (7.5 mg/ml), in the synovial fluid (3-4 mg/ml), in umbilical cord (3 mg/ml), in the vitreous of the eye (0.2 mg/ml) and in skin (0.5 mg/ml). In other tissues that contain less hyaluronan, it forms an essential structural component of the matrix. In cartilage it forms the aggregation centre for aggrecan, the large chondroitin sulfate proteoglycan, and retains this macromolecular assembly in the matrix by specific hyaluronan-protein interactions. It also forms a scaffold for binding of other matrix components around smooth muscle cells on the aorta and on fibroblasts in the dermis of skin. The largest deposit of hyaluronan resides in the skin with about 8 g of an adult human. Hyaluronan has also been detected intracellularly in proliferating cells.

Hyaluronan consists of basic disaccharide units of D-glucuronic acid and D-N-acetylglucosamine. They are linked together through alternating beta-1,4 and beta-1,3 glycosidic bonds. The number of repeat disaccharides, in a completed hyaluronan molecule can reach 10,000 or more and a molecular mass of ˜4 million daltons (each disaccharide is ˜400 daltons). In a physiological solution, the backbone of a hyaluronan molecule is stiffened by a combination of the chemical structure of the disaccharide, internal hydrogen bonds, and interactions with solvent. A hyaluronan molecule assumes an expanded random coil structure in physiological solutions which occupies a very large domain. The actual mass of hyaluronan within this domain is very low, and ˜0.1% molecules would overlap each other at concentrations of 1 mg hyaluronan per ml or higher.

Many different functions have been assigned to hyaluronan. They can be grouped into cellular, physiological and pathological functions. Most of the functions are determined by the physical properties or by interactions with hyaluronan, binding proteins. A prominent physiological function of hyaluronan is the creation of hydrated pathways that allow the cells to penetrate cellular and fibrous barriers. Such hydrated pericellular matrices are not only required for cell rounding in mitosis, but also for cell migration during morphogenesis and wound healing. In cartilage hyaluronan is the key element that holds the proteoglycans together to form large aggregates. A hyaluronan binding domain at the C-terminus of aggrecan is responsible for this aggregation.

Hyaluronan synthesis in mammalian cells differs from other polysaccharides in many aspects. It is elongated at the reducing end by alternate transfer of UDP-hyaluronan to the substrates UDP-GlcNac and UDP-GlcA liberating the UDP-moiety [22]. Other glucosaminoglycans grow at the non-reducing end and require a protein backbone. Hyaluronan is synthesized at plasma membranes and it was speculated that nascent chains are directly extruded into the extracellular matrix [3]. In contrast, other glucosaminoglycans are made in the Golgi. Chain initiation does not require a protein backbone as for proteoglycans, nor preformed oligosaccharides as starters, only the presence of the nucleotide sugar precursors are sufficient to initiate new chains. During elongation the chain is retained on the membrane integrated synthase. This mechanism of synthesis operates for the synthesis in vertebrates and in gram-positive Streptococci.

Research on hyaluronan synthesis is greatly facilitated by the use of streptococci due to the ease of cultivation and the quantity of material. Hyaluronan is produced by group A and C streptococci and deposited in a capsule [35]. The hyaluronan capsule is the major virulence factor of such pathogenic streptococci [36-38]. It protects the bacteria from phagocytosis [39] and from oxygen damage [40]. Group A and C streptococci differ in their capacity to retain hyaluronan as a coat on their cell surface [41]. In group C streptococci a 56 kDa hyaluronan receptor was closely associated to the synthase. This protein had an intrinsic kinase activity that performed autophosphorylation in response to extracellular ATP. Autophosphorylation of the 56 kDa protein led to a reduction of hyaluronan binding and increased shedding of the hyaluronan capsule. Simultaneously, the synthase increased its activity to replace the lost hyaluronan chains. A large hyaluronan chain thus appears to inhibit its own elongation, when it was retained in the vicinity of the synthase. The hyaluronan synthase is a membrane protein that could be purified by a new method in active form [42].

Three genes comprising the has operon have been shown to encode enzymes that are involved into the synthesis of hyaluronan in Streptococci: hasA for the hyaluronan synthase; hasB for the UDP-glucose dehydrogenase, which synthesize glucuronic acid from UDP-glucose; hasC for the UDP-glucose phosphorylase, which forms UDP-glucose from UTP and glucose-1-phosphate. Most bacterial polysaccharides studied so far exit cells by specialized transporters. Genes encoding these transporter proteins are often found in the next vicinity of the operon that encodes enzymes utilized in the synthesis of the polysaccharide. The upstream chromosomal region of S. pyogenes flanking the has operon was partially analysed [43;44]. However, although the authors found a gene cluster containing an ABC transporter they did not inactivated the ABC transporter itself and postulated that hasA and hasB were sufficient for the capsule formation by streptococci. In another investigation irradiation inactivation was employed to calculate the size of the capsule producing proteins [45]. From this study the authors concluded that the size excluded the participation of proteins other than the synthase itself. The aim of our work was to further investigate the hyaluronan export from Streptococci.

Construction of a S. pyogenes Mutant Library

We constructed a mutant library by chromosomal integration of the plasmid pGhost9:ISS1 harbouring an insertion element ISS1 for gene inactivation [64]. We used the Streptococcus pyogenes M49 strain CS101 as a host that produced high amounts of hyaluronan and that possessed large mucoid colonies when cultured on blood agar.

### Generation of Hyaluronan Deficient Mutants

Isolation of hyaluronan deficient mutants is performed by visual appearance of the colonies. Glossy colonies produce the hyaluronan capsule while opaque colonies lack such capsules [65]. For group A streptococci this difference is only seen on blood agar plates, because whole blood contains components that repress the csrR/csrS regulators responsible for inhibition of has gene expression [66-68]. Among the clones with reduced mucoid appearance we found one with an unaffected synthase activity, but with reduced hyaluronan release into the medium (FIG. 2) and capsule production (FIG. 3). This mutant was selected for further characterization.

The bacterial viability of the wildtype and mutant strains was tested by vital staining. The number of dead cells after 1 and 2 hrs were 10% and 20%, respectively, and remained constant for the next 4 hrs (data not shown). This result suggested that also residual hyaluronan production of the mutant could be due to leakiness of dead bacteria.

### Isolation and Characterization of Insertion Mutants

The site of integration was determined by cloning the insertion site in E. coli and sequence analysis. The sequences were compared with the known sequence of Streptococcus pyogenes M1 (strain SF370) using the data base of the University of Oklahoma Advanced Center for Genome Technology. All the genes identified in the mutant strain were at least 99% homologous to those of the sequenced M1 strain. Among mutants with inserts into the has gene cluster, we found a mutant with an insert into a gene that displayed strong homology to ABC transporters. The homologous gene cluster within the Streptococcus pyogenes M1 (strain SF370) chromosome was located in the immediate vicinity of the has gene cluster (FIG. 4). This insertion site of the vector was confirmed by PCR using primers annealing to vector sequences and genomic nucleotide sequences upstream or downstream of the hax locus followed by the sequencing of the PCR products. The cistron contains seven open reading frames (ORFs) that are all transcribed in the same direction in opposition to the transcription direction of the has gene cluster: ORF 1 encodes an unknown protein with some homology to RecA protein; ORFs 2 and 3 encode for proteins with some homology to zinc proteases; ORF 4 encodes a CDP-phosphatidylglycerol-glycerophosphate transferase; the next two ORFs, ORF 5 and 6 (where the transposon insertion was found) belong to members of an ABC transporter family (ATP binding proteins); ORF 7 encodes a putative integral membrane protein with five potential transmembrane regions; ORF 8 was found to have homology with a secretory protein SAI-B from Staphylococcus aureus and also to contain a putative membrane spanning region.

### Rescue of Hyaluronan Release by Streptococci by Genetic Complementation

A 3.8 kb chromosomal fragment comprising haxA to haxD was amplified by PCR and subcloned into pAT28 and the construct pAT28hax was obtained. When pAT28hax was transfected into the hax mutant, colonies displayed the mucoid phenotype on agar plates, while bacteria transfected with pAT28 only resembled the original mutant colonies. The rate of hyaluronan release of the cells transformed with pAT28hax almost reached the level of the wild type cells (FIGS. 2 and 3). The results indicated that an ABC transporter was required for hyaluronan capsule production in intact streptococci. We have named the genes encoded in ORF 5 to ORF8 haxA, haxB, haxC and haxD.

### Conclusion

In contrast to the existing paradigm, we have identified an ABC transporter that is required for hyaluronan extrusion through the cell membrane in group A Streptococcus pyogenes. The involved proteins are encoded at a chromosomal region immediately adjacent to the has genes responsible for hyaluronan synthesis. They include four ABC transporter proteins that we have named haxA, haxB, haxC and haxD. Both haxA and haxB contain an ATP binding cassette, the C-motif and a Walker-A motif that are characteristic for ABC-transporter. Insertional mutagenesis produced a mutant that was not mucoid. The mucoid phenotype was rescued by transfection with DNA encoding the four transporter genes.

To much of our surprise, we discovered that the bacterial export system showed high homology to human ABC transporters, among which the multidrug resistance transporter (MDR or ABCB) and the multidrug resistant associated proteins (MRP of ABCC) are prominent members. FIG. 5 shows the phylogenetic relationship of Hax-A (also referred to as Spy2194) and Hax-B (also referred to as Spy2195) with human ABC transporters as calculated by the Custal method. HaxA and haxB are related to each other and to the human ABCB transporter, followed by ABCC (MPR) transporter.

Human ABC transporter are a protein family of 49 transporter that are responsible for the transport of many substrates. Many of them have a very broad substrate specificity. They are grouped in 7 subfamilies: ABCA or ABC1; ABCB or MDR; ABCC or MRP; ABCD or ALD; ABCE or OABP; ABCF or GCN20; ABCG or White (FIG. 1). The most important and best studies member is the P-glycoprotein that is expressed in many tissues. It exports chemostatic drugs out of tumour cells and thus makes the cells resistant towards drug treatment. However, the physiological functions of most transporters is still elusive [48].

The pharmaceutical industry spends much effort to develop improved inhibitors for these transporters [49], and therefore many inhibitors for ABC-transporters are available [50-54]. A classical inhibitor of the first generation is Verapamil that often serves as a reference compound. It is a drug that specifically inhibits the slow Na—Ca-channel and is applied under conditions of myocardial infarct and disturbances of heart rhythm (supraventricular tachycardy) that is caused by arteriosclerosis or impaired heart function. It influences Ca-influx and electromechanical coupling of smooth muscles and can induce a rapid drop in blood pressure. It has also a long lasting dilatory effect. It lowers arterial blood pressure and protects from increased permeability in the microvascular system [55-57]. Similar effects have been described for other inhibitors of the first generation: Quinidine; Chlomipramine; Chloroquine; Quinine; Emitine; Dilthiazem; Nicardipine; Nifedipine; Bepridil; Amiloride; Cyclosporin; Rapamycin, Reserpin. In all cases the mechanisms of action is unknown. An inhibition of hyaluronan production by these drugs has not been described.

The application of Verapamil and other MDR inhibitors of the first generation in oncology was not very successful. Therefore many companies search for improved inhibitors [53;58]. A series of such drugs are currently in clinical trails, e.g. Valspodar or PSC833 from Novartis. It is a member of the immuno suppressive drugs such as Cyclosporin A and Rapamycin. The inhibitors such as Valspodar and Verapamil primarily inhibit MDR (ABC-B1) transporter, but also act on MRP transporter [54]. Their effects on ABCA transporters are rather low. DIDS (4,4-diisothiocyanatostilbene-2,2-disulfonic acid) is a specific inhibitor of the ABC-A1 transporter and it does not inhibit the ABCB1 transporter [60]. Another specific ABCA transport inhibitor is glyburide [52]. The ABCC (MRP) transporter are reknown as organic anion transporter [50;51;54;61;62]. It can be inhibited by the general anion inhibitor benzbromarone or by the specific ABCC1 inhibitor MK-571.

We performed a series of experiments to investigate whether ABC transporters are also responsible for the export of hyaluronan from eukaryotic cells. ABC transporter inhibitors were employed to assay for inhibition of hyaluronan production in cell culture. Because hyaluronan synthesis is also required for detachment during mitosis and growth of the human HT1080 cell line [69], we also analysed the effect on cell proliferation.

### ABCB1 (MRP) Inhibitors

Human skin fibroblasts were grown in cell culture in the presence of increasing concentrations of the ABCB1 inhibitors Verapamil and Valspodar. The amount of hyaluronan in culture medium and the cell number were determined after 3 days. FIG. 6 shows that both Verapamil and Valspodar reduced the hyaluronan transport as well as cell growth. Valspodar reduced hyaluronan transport more efficiently than Verapamil. The effective concentrations were similar to those used for the inhibitions of multidrug resistance.

Because the inhibition of hyaluronan synthesis could also be caused indirectly through the inhibition of growth, the effect of Verapamil and Valspodar was also studied on human synovial fibroblasts that were expected to produce hyaluronan also under non-proliferating conditions. FIG. 7 shows that Verapamil inhibited growth as well as hyaluronan production. In contrast, Valspodar only inhibited hyaluronan production, but not cell proliferation.

Since the effect of Verapamil and Valspodar could also be due to toxic effects on cellular metabolism, their influence on the hyaluronan transport in isolated membranes from human skin fibroblasts was analysed. FIG. 8 shows that both inhibitors blocked the hyaluronan transport in a concentration dependent manner in membranes of human skin fibroblasts.

Furthermore, we used a monoclonal antibody against P-glycoprotein (C219 from Calbiochem) [70] in order to verify the participation of the MDR transporter in hyaluronan export by another method. Membranes from this cell line were incubated with and without the antibody and then assayed for hyaluronan transport. The antibody decreased the hyaluronan transport activity by 20%.

The above results suggested that the ABCB1 (MRP) transporter was involved in hyaluronan export. This finding was confirmed by comparing the effect of Verapamil on hyaluronan transport in the human colon carcinoma cell lines HT29 and HT29-mdr which differ only in the expression of the multidrug resistance protein P-glycoprotein [71;72]. FIG. 9 shows that increasing concentrations of Verapamil more efficiently blocked hyaluronan transport in HT29 than in HT29-mdr.

Furthermore, Verapamil was tested for its effect on the hyaluronan transport activity in isolated membranes from HT29 und HT29-mdr. FIG. 10 shows that Verapamil does not inhibit the transport in membranes from the drug resistant cell line. In the sensitive cell line, inhibition is not complete. It is therefore possible that further transporters are involved in hyaluronan export. This hypothesis was investigated by the following experiments.

### ABCA Inhibitors

DIDS (4,4-diisothiocyanatostilbene-2,2-disulfonic acid) is a specific inhibitor of the ABC-A transporter and it does not inhibit the ABCB transporters [60]. Its effect was assayed on membranes from a human fibroblast cell line. It reduced the hyaluronan transport activity by 70%.

Another specific ABCA transport inhibitor is glyburide [52]. Its efficiency was compared with Valspodar for the hyaluronan production in a human fibroblast cell line. FIG. 11 shows that glyburide partially inhibited hyaluronan production, but not as efficient as Valspodar. This experiment also indicated that the inhibitory effect of Valspodar on the hyaluronan production in intact cells was stronger than on the hyaluronan transport in membranes.

### ABC-C (MRP)

MRP transporter can be inhibited by benzbromarone or MK-571. These drugs were analysed for their effects on the hyaluronan transport activity in membranes from human fibroblasts and compared with Valspodar. FIG. 12 shows that benzbromarone almost completely inhibited the hyaluronan synthase activity, whereas Valspodar and MK-571 were less efficient.

The results described above lead to the conclusion that more than one transporter could be involved in hyaluronan secretion from human cells and may function simultaneously in a given cell: ABC-B (MDR) transporter, ABCA transporter, and ABCC (MRP) transporter. Accordingly, in a preferred embodiment, said human ABC-transporter(s) is(are) a member of the human ABC-B (MDR)-subfamily, the ABC-A subfamily and/or the human ABC-C (MRP)-subfamily.

The hypothesis of multiple transporters for hyaluronan may appear surprising at first glance; however, comparing the streptococcal proteins haxA and haxB with their human counterpart also indicated that they have great homology to two different human ABC transporters ABCB and ABCC, respectively.

These results demonstrate for the first time that ABC transporter inhibitors are able to inhibit hyaluronan secretion as mediated by ABC-transporter(s) in eukaryotic cells. Many of such inhibitors have already been used and tested as drugs to treat human cancer patients (described herein elsewhere). But there exists no report on inhibition of hyaluronan production. Overproduction of hyaluronan is, however, a central problem in many diseases (i.e. in diseases which are associated with an excess transport of hyaluronan across a lipid bilayer as described herein), in particular in ischemic or inflammatory edema or in arthritis. Also many human tumors are characterized by an overproduction of hyaluronan such as melanoma [89], mesothelioma [117] or colon carcinoma [118]. Because hyaluronan production is corellated with cell proliferation [86], inhibition of hyaluronan transport will also reduce tumor growth. Overproduction of hyaluronan is also the cause of lump formation after contusion or insect bites, therefore is will be possible to inhibit swelling by the inhibitors of hyaluronan transport. In fact, most injuries are followed by inflammation and hyaluronan overproduction that may lead to severe health problems such as organ transplantation and tissue rejection, hyaluronan production after a heart infarct, alveolitis, pancreatitis, pulmonary or hepatic fibrosis, radiation induced inflammation, Crohn's disease, myocarditis, scleroderma, psoriasis, sarcoidosis [119-135]. In particular, the unbalanced hyaluronan and proteoglycan synthesis by chondrocytes in arthritis appears to be the first biochemical event that eventually leads to complete joint destruction.

Accordingly, it has to be understood that the present invention, in general, relates to the use of at least one inhibitor of at least one ABC-transporter capable of transporting hyaluronan across a lipid bilayer, for the preparation of a pharmaceutical composition for the treatment of a disease which is associated with an excess transport of hyaluronan across a lipid bilayer. The term “excess transport” in this context means that the transport of hyaluronan as mediated by ABC-transporter(s), exceeds the transport level as compared with a normal/natural state of a comparable control-cell/subject. In this regard, “exceeds the transport level” does not only mean that the actual transport rate is increased but also that the respective ABC-transporter(s) which is(are) responsible for this excess transport are improperly regulated. For example the ABC-transporter(s) are active (i.e. they transport hyaluronan across a lipid bilayer) although they should be silent/inactive. It is, for example, possible that the hyaluronan transport as mediated by the respective ABC-transporter(s) is not terminated after induction, i.e the ABC-transporter(s) go on to transport hyaluronan to the exterior of a cell. In all such cases it is necessary to effect the ABC-transporter(s) with the inhibitors as described herein. The term “normal/natural state of a comparable control-cell/subject” is explained herein elsewhere. Thus, it is now possible to treat/ameliorate and/or prevent diseases which are associated with an excess transport (mediated by ABC-transporter(s)) of hyaluronan. The skilled person is well aware which specific diseases are characterized by an excess level of hyaluronan at the exterior of cells and, provided with the teaching and disclosure of the present invention, can easily test for such an excess hyaluronan transport, e.g. by use of the methods as disclosed herein. Thus, it is now possible to identify a subject at risk for a disease which is associated with an excess transport of hyaluronan across a lipid bilayer or to diagnose a disease which is associated with an excess transport of hyaluronan across a lipid bilayer, inter alia by the identification of an over-expression (nucleic acid level or protein level) of ABC-transporter(s) and/or the identification of an increased transport of hyaluronan as mediated by ABC-transporter(s). In accordance with this embodiment, the diagnosis can, e.g., be effected by isolating cells from an individual. Such cells can be collected from body fluids, skin, hair, biopsies and other sources as described herein elsewhere.

It is envisaged that all the uses and/or methods described herein below and/or above for arthritis (which is a disease within the meaning of the present invention, i.e. which is associated with an excess transport of hyaluronan across a lipid bilayer) apply mutatis mutandis to other diseases/conditions which are associated with an excess transport of hyaluronan across a lipid bilayer. Such diseases/conditions can be exemplified as follows: ischemic or inflammatory edema; tumors which are characterized by an overproduction of hyaluronan such as melanoma [89], mesothelioma [117] or colon carcinoma [118]; lump formation after contusion or insect bites; injuries/conditions which are followed by inflammation and hyaluronan overproduction like heart infarct, alveolitis, pancreatitis, pulmonary or hepatic fibrosis, radiation induced inflammation, Crohn's disease, myocarditis, scleroderma, psoriasis, sarcoidosis [119-135]. Suppression of hyaluronan production can be beneficial for the diseases mentioned above.

Ischemic edema are caused by increased hyaluronan production around blood vessels that leads to vessel constriction and reduced oxygen supply. This is thought to be the main cause of death after an heart attack. Therefore immediate application of drugs inhibiting hyaluronan production will improve these conditions. Tissue necrosis will lead to inflammation with increased hyaluronan production that in turn causes edema. To break this vicious cycle inhibitors of hyaluronan transport will expand the choice for medical treatments.

Lump formation after contusion or insect bites can be life threatening, if for instance a bee-sting occurred in the throat. The swelling may suffocate the victim. Many metastatic tumors overproduce hyaluronan or stimulate the surrounding stroma to produce hyaluronan. The swollen tissue enables the metastatic tumor cells to easily invade the tissue. Therefore inhibition of hyaluronan transport will decrease the metastatic potential [136-140]. In addition hyaluronan production is also required for proliferation of fibroblasts [69]. Therefore inhibition of hyaluronan transport will also reduce the growth of tumors.

Thus, the present invention relates e.g. to the use of at least one inhibitor of at least one ABC-transporter capable of transporting hyaluronan across a lipid bilayer, for the preparation of a pharmaceutical composition for the treatment of a disease which is associated with an excess transport of hyaluronan across a lipid bilayer, e.g. arthritis.

It must be noted that as used herein, the singular forms “a”, “an”, and “the”, include plural references unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

The term “inhibitor” defines in the context of the present invention a compound or a plurality of compounds which interact(s) with one or more ABC-transporter(s) such that the hyaluronan transport mediated by such ABC-transporter(s) is reduced. It is envisaged that the inhibitors of the present invention are capable to reduce or abolish the transport of hyaluronan mediated by one or more ABC-transporter(s) across a lipid bilayer in a cell/subject in a way which is sufficient to reduce the hyaluronan-transport to at least about the same level as compared to a normal/natural state of a comparable control-cell/subject. The meaning of the “comparable control-cell/subject” is further defined herein below. The term “plurality of compounds” is to be understood as a plurality of substances which may or may not be identical. The plurality of compounds may preferably act additively or synergistically. Said compound or plurality of compounds may be chemically synthesized or microbiologically produced and/or comprised in, for example, samples, e.g., cell extracts from, e.g., plants, animals or, microorganisms. Furthermore, said compound(s) may be known in the art but hitherto not known to be capable of reducing the transport of hyaluronan mediated by at least one ABC-transporter.

The interaction of the inhibitor with one or more ABC-transporter(s) such that the hyaluronan transport mediated by such ABC-transporter(s) is reduced can, in accordance with this invention e.g. be effected by a reduction of the amount of the ABC-transporter(s) in cells, in tissues comprising said cells or subjects comprising said tissues or cells for example by aptamers, antisense oligonucleotides, iRNA or siRNA which specifically bind to the nucleotides sequences encoding said ABC-transporter(s) or by ribozymes which specifically degrade polynucleotides which encode ABC-transporter(s)); by blocking the binding site of the ABC-transporter(s) for hyaluronan; by competitive or allosteric inhibition of the hyaluronan transport mediated by the ABC-transporter(s) or by otherwise reducing or preventing the transport of hyaluronan mediated by one or more of the ABC-transporter(s), for example by directing antibodies and/or aptamers to such ABC-transporter(s) as defined herein and thereby reducing or preventing the hyaluronan transport mediated by said ABC-transporter(s). Thus, an example of an inhibitor of this invention is an antibody, preferably an antibody the binding of which interferes with the transport of hyaluronan mediated by the ABC-transporters of this invention; an antisense construct, iRNA, siRNA or ribozyme constructs directed against a transcript or the coding nucleotide sequence of the ABC-transporter(s) of the invention; nucleotide sequences encoding such constructs and compounds which inhibit the transport of hyaluronan mediated by the ABC-transporter(s) as defined herein, for example by blocking or disrupting the binding site of the ABC-transporter(s) for hyaluronan or by allosteric or competitive inhibition of the hyaluronan transport mediated by the ABC-transporter(s) are defined herein.

Such inhibitors as mentioned herein are explained and discussed in more detail throughout the specification. Furthermore, the present invention provides numerous screening methods, test-systems and assays which allow the skilled person to screen for inhibitors as defined herein and to test the effect/effectiveness of such inhibitors. Such test systems are explained in great detail e.g. in the appended examples.

The term “reduced” or “reducing” as used herein defines the reduction of the hyaluronan transport across a lipid bilayer, preferably to at least about the same level as compared to a normal/natural state of a comparable control-cell/subject. Accordingly, it is understood that the reduction mediated by the inhibitor aims at “normalizing” the transport activity of ABC-transporter(s) of hyaluronan across a lipid bilayer, which does, however, not exclude that the inhibitor as defined herein might also reduce the hyaluronan transport across a lipid bilayer to a lower level as compared to a normal/natural state of a comparable control-cell/subject. It is also envisaged that the inhibitor totally abolishes the transport of hyaluronan when compared to a normal/natural state of a comparable control-cell/subject. The term “normal/natural state of a comparable control-cell/subject” means the transport-rate of hyaluronan as mediated by one or more ABC-transporter(s) in a control-cell which is preferably of the same nature as the test-cell (e.g. both cell are chondrocytes) but which is derived from a different source. “A different source” includes e.g. a cell/tissue sample obtained from a healthy subject which does not suffer from a disease which is associated with an excess transport of hyaluronan across a lipid bilayer, e.g. arthritis (or other diseases described herein) or a cell/tissue sample obtained from a distinct joint of the same subject wherein said different joint appears to be free from associated symptoms of a disease which is associated with an excess transport of hyaluronan across a lipid bilayer, e.g. arthritis. Assays and histological methods to classify a disease which is associated with an excess transport of hyaluronan across a lipid bilayer, e.g. arthritis (cartilage destruction associated with arthritis) are well-known to the skilled person and in addition outlined in the appended examples. However, even in cases where the inhibitor will not reduce the hyaluronan-transport across a lipid-bilayer to the normal/natural state of a comparable control-cell/subject but actually reduces the hyaluronan transport when compared to the transport rate before the addition of said inhibitor, it will be appreciated that said inhibitor has a beneficial effect on the cell/tissue/subject in question.

For medical treatment it is preferable to use inhibitors that act in a reversible manner and do not block biochemical processes completely, because such drugs can be applied in a dosage that complies with the desired effect.

Accordingly, it is envisaged that the inhibitor of the invention at least reduces the hyaluronan synthesis/transport rate as mediated by at least one ABC-transporter to 5%, 10%, 20%, 30%, 40%, 50%, 60%, 70%, 80%, 90% or even 100% when compared to synthesis/transport the transport rate that is achieved without the addition of said inhibitor. One specific screening assay for the hyaluronan transporter is based on the extrusion of labelled hyaluronan oligosaccharides from intact cells in monolayer culture. Said assay is further explained herein below as well as in the appended examples (e.g. Example 8 or Example 11). In such cases it is sufficient to analyse the effect of the inhibitor e.g. on a cell comprising one or more ABC-transporter(s), i.e. one compares the hyaluronan-transport before and after the addition of the inhibitor and thereby identifies inhibitors which reduce the transport-rate of hyaluronan across a lipid bilayer. Thus, inhibitors which exert a reduction of the hyaluronan transport across a lipid bilayer are within the scope of this invention as long as they exert a beneficial effect which means e.g. a reduction of the overall transport of hyaluronan across a lipid-bilayer; reduction of the destruction of cartilage; maintenance of the actual state of destruction of cartilage; prevention of a further destruction of the cartilage etc. restoring the healthy condition after an injury or damage. There are a number of techniques that enable the assessment of efficiency. These techniques can be grouped into “process” measures and “outcome” measures. Products of both synthesis and degradation of cartilage can be assayed in the synovial fluid. Joint physiology can be imaged using techniques such as scintigraphy, which reflect regional blood flow and bone activity, and MRI, which will reveal the water content of the tissue. The outcome measures can be divided into four main categories—anatomical change (plane radiograph, other images), pain and stiffness, joint function (range of movement, stability), disability [90].

The reduction will also depend on the dosage and on the way of administration of the inhibitor. The dosage regimen utilizing the inhibitor of the present invention is therefore selected in accordance with a variety of factors including type, species, age, weight, sex and medical condition of the patient; the severity of the condition to be treated; the route of administration; and the particular compound employed. It will be acknowledged that an ordinarily skilled physician or veterinarian can easily determine and prescribe the effective amount of the compound required to prevent, counter or arrest the progress of the condition. Test-systems which are suitable for such purposes, i.e. which allow to measure the effect of an inhibitor on the hyaluronan-transport are described herein.

It is preferred that for the ABC-transporter(s) as described herein, the inhibitors of the invention have an IC50 between about 10 nanomolar and about 300 micromolar.

The term “capable of transporting hyaluronan across a lipid bilayer” means that the ABC-transporter(s) as described herein is(are) able to transport hyaluronan as defined herein from one compartment to another compartment, wherein both compartments are separated by a lipid bilayer. The term “lipid bilayer” is well-known to the skilled person [91] and denotes e.g. biological membranes or liposomes. Assay and test-systems which allow the determination of hyaluronan-transport across a lipid bilayer are explained in the appended examples in great detail. It will be understood that the term “capable of transporting hyaluronan across a lipid bilayer” defines in the context of cells or tissues comprising said cells, the transport of hyaluronan to the exterior of the cell (e.g. the extracellular milieu).

It has to be understood that in the context of the present invention, the term “at least one inhibitor” or “at least one ABC-transporter(s)” comprises at least one, at least two, at least three, at least four, at least five, at least six . . . etc. inhibitor(s)/ABC-transporter(s). In some cases it will be sufficient if the inhibitor interacts with, and thereby inhibits only one ABC-transporter which is present in a cell/tissue in question and which is able to transport hyaluronan across a lipid bilayer, although other ABC-transporter(s) are present (regardless whether these additional ABC-transporter(s) are able to transport hyaluronan or not). It will be understood that in such particular cases, the inhibition of one ABC-transporter is sufficient to reduce the transport of hyaluronan across a lipid bilayer to such an extent that the overall transport of said cell is reduced to the same/or below the level as compared to a normal/natural state of a comparable control-cell/subject or at least to such an extent which exerts a beneficial effect to the cell/tissue/subject (beneficial effect can be e.g. reduction of the overall transport of hyaluronan across a lipid-bilayer; reduction of the destruction of cartilage; maintenance of the actual state of destruction of cartilage; prevention of a further destruction of the cartilage etc; prevention of fluid accumulation in edema, prevention of tissue softening that is required for invasion of inflammatory cells or for metastasis, reduction of cell growth of tumors).

In other cases, it is envisaged that the inhibitor interacts with and thereby inhibits more than one ABC-transporter which is present in the cell/tissue/subject in question and which are able to transport hyaluronan across a lipid bilayer. In such cases it might be desirable to use e.g. at least two inhibitors (or more) which might act additive or synergistically. Said two or more inhibitors will be selected in accordance with the specific conditions which are to be treated, i.e. it might be desirable to combine two or more inhibitors which are distinct with respect to the hydrophobicity, molecular mass, antigenic characteristics, route of administration, half-life in blood or serum etc. Alternatively, the inhibitor itself can be constructed to encompass two or more different entities which are able to inhibit the transport of hyaluronan across a lipid bilayer (by way of inhibiting one or more ABC-transporter(s)) and which are linked for example via peptide bonds or other suitable linkers like, e.g. chemical bonds. Suitable methods/linkers to connect two or more compounds are well-known in the art. In such a particular case one inhibitor (which actually consists e.g. of two different inhibiting entities) is e.g. able to inhibit e.g. two or more different ABC-transporter(s) which are able to transport hyaluronan across a lipid bilayer. It will be understood that the number of inhibitors and or the number of ABC-transporter(s) which are to be inhibited by said inhibitors will be selected on a case to case basis in order to provide a suitable treatment for the cell/tissue/subject. In this context, “suitable” means that the treatment with the respective inhibitor(s) of the invention exerts a beneficial effect, e.g. it prevents, counters or arrests the progress of the condition (e.g. reduction of the overall transport of hyaluronan across a lipid-bilayer; reduction of the destruction of cartilage; maintenance of the actual state of destruction of cartilage; prevention of a further destruction of the cartilage etc.). The number of inhibitors and or the number of ABC-transporter(s) which are to be inhibited by said inhibitors by utilizing the inhibitors of the present invention for treating or preventing a disease which is associated with a disease which is associated with an excess transport of hyaluronan across a lipid bilayer, e.g. arthritis (described elsewhere in this specification), is selected in accordance with a variety of factors including type, species, age, weight, sex and so on.

In a preferred embodiment of the use or the methods of the present invention said inhibitor(s) specifically reduce(s) the transport of hyaluronan across a lipid bilayer mediated by at least one of said ABC-transporter(s). The term “specifically reduce(s)” used in accordance with the present invention means that the inhibitor specifically causes a reduction of the transport of hyaluronan as mediated by ABC-transporter(s) but has no or essentially has no significant effect on other cellular proteins or enzymes. It will be understood that the inhibitor(s) of the present invention may also have an influence on the hyaluronan-synthase, e.g. the activity of the hyaluronan-synthase is reduced or abolished by the respective inhibitor(s). Alternatively, the inhibitor has no or essentially no influence on the hyaluronan-synthase, which means that the hyaluronan-synthase activity is not reduced or only reduced by 5%, 10%, 15%, 20%, 25%, 30%, 35% or 40% and so on. The specificity of the inhibitor, e.g. with respect to the hyaluronan-synthase or the ABC-transporter(s) can be measured e.g. by expression of the synthase or ABC-transporter in a suitable cell which, as such, is deficient of the corresponding hyaluronan-synthase or ABC-transporter (e.g. a bacterial cell). The inhibitors can also be discriminated by virtue of their binding to the respective proteins. Synthase inhibitors such as peroidate oxidized nucleotide sugars will bind to the synthase and inhibitors of hyaluronan transport will bind to ABC-transporter(s) [24,25]. Methods have been described that assay the binding of inhibitors to ABC transporters [92;93]. Indirectly acting inhibitors that change the functional activity of the synthase or the transporter will bind to neither one, but to intracellular signaling factors. One specific screening assay for the hyaluronan transport as mediated by the ABC-transporter(s) is based on the extrusion of labeled hyaluronan oligosaccharides from intact cells in monolayer culture (see e.g. Example 11). Alternatively, liposomes can be employed which encompass one or more ABC-transporter(s) in the lipid bilayer. For this assay, test-compounds as described herein elsewhere, e.g. labeled hyaluronan oligosaccharides can be introduced into the cytosol of cells or into the liposomes. Because these test-compounds will normally not transverse the plasma membranes/lipid bilayer, they are introduced e.g. by osmotic lysis of pinocytotic vesicles according to a method that has already successfully been applied for the introduction of periodate oxidized nucleotide sugars [25]. Alternatively, it is possible to introduce the test-compounds by other suitable methods like electro-chemical-poration; lipofection; bioballistics or microinjection (these methods are well-known in the art). Hyaluronan oligosaccharides are prepared from commercially available hyaluronan by digestion with hyaluronidase and sized fractionation by gel filtration as described [102]. Appropriate oligosaccharide fractions having a length between 2 and 50 disaccharide units are labeled by incorporation of a biotin, radioactivity, or a fluorescent probe. These methods are routine published procedures [87,99-101,103]. For example the cells are seeded into multiwell microtiter plates to a density of at least 4×10⁴ cells/cm². When the cells are attached to the plastic surface after a few hours, they are washed with phosphate buffered saline and incubated with the labeled hyaluronan dissolved in medium for osmotic lysis of pinocytotic vesicles (growth medium such as Dulbeccos medium containing 1 M sucrose, 50% poly(ethylene glycol)-1000) for at least 5 min up to several hours at 37° C. During this time the cells will pinocytose this hyperosmotic medium and the labeled hyaluronan. The above medium is substituted by a mixture of Dulbeccos medium and water (3:2) for 2 min. This causes the intracellular pinocytotic vesicles to lyse and to liberate the contents into the cytosol without damaging the cells. The cells can be subjected to this incubation sequence several times. The cells are washed thoroughly several times with phosphate buffered saline or growth medium to remove extracellular labeled hyaluronan and are then ready for the assay. They are incubated in growth medium containing the compound to be tested in different concentrations for several hours. During this time the labeled hyaluronan will be transported back into the medium. The amount of labeled hyaluronan oligosaccharide in the medium can be determined by a biotin-related assay, by radioactivity or by fluorescence intensity.

It will be understood that the above method is only an exemplary method. Other methods which are also within the gist of the present invention are described herein below as well as in the appended examples.

In a preferred embodiment, said inhibitor “specifically reduces” only such ABC-transporter(s) which are able to transport hyaluronan across a lipid-bilayer but does not or essentially does not inhibit other ABC-transporter(s), which are not able to transport hyaluronan across a lipid bilayer. Test-systems and methods for measuring the specific transport of hyaluronan across a lipid bilayer as well as methods for analyzing the transport of hyaluronan mediated by an ABC-transporter are well known to the skilled person and also provided in the appended examples. For example ABC transporters can specifically be identified as described above with the MDR transporter in HT29 and HT29-mdr cells. The gene for the transporter is transfected and the protein is expressed in a given cell line and the hyaluronan transport activities are compared (of the parent and the transfected cells) towards the responsiveness of the inhibitors.

Only those inhibitors that inhibit the ABC-transporter(s) of interest (i.e. ABC-transporter(s) which are able to transport hyaluronan across a lipid bilayer) but do not or do not essentially bind to any of the other ABC-transporter(s) which are preferably expressed by the same cell or tissue are considered to be specific for the ABC-transporter(s) of interest (i.e. ABC-transporter(s) which are able to transport hyaluronan across a lipid-bilayer).

In another embodiment of the uses of the present invention, said ABC-transporter(s) is(are) a mammalian ABC-transporter(s).

In a preferred embodiment said mammalian ABC-transporter(s) is(are) a human ABC-transporter(s).

The ABC superfamily is one of the largest families of proteins. The most recent annotation of the human genome sequence revealed 49 genes for ABC proteins. The ABC-transporters were grouped into seven sub-classes, ranging from ABCA to ABCG [see e.g.: http://nutrigene.4t.com/humanabc.htm] based on genomic organization, order of domains and sequence homology. The known 49 human ATP-transporter(s) including their Accession numbers are depicted below as well as in FIG. 1.

49 HumanATP-Binding Cassette Transporters

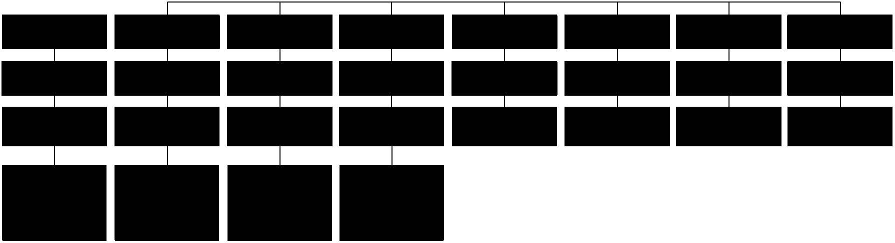

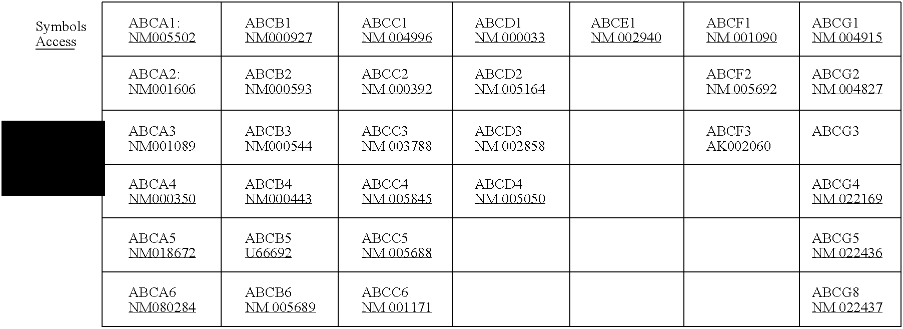

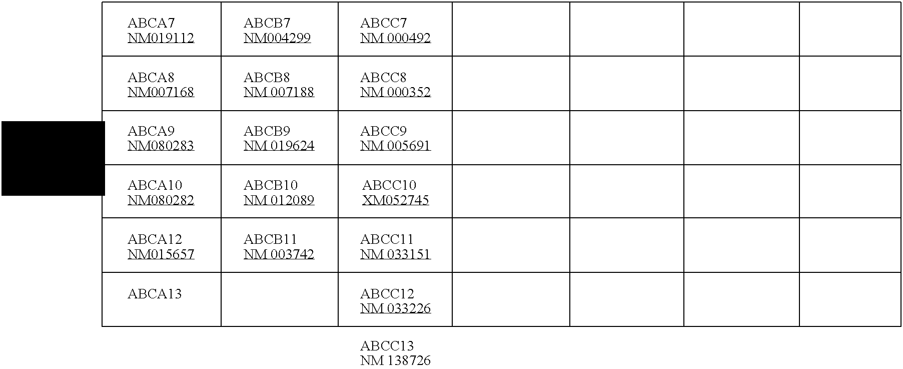

It will be understood that the sequences of the indicated ABC-transporter(s) may also deviate from the above indicated accession sequences. It is therefore envisaged that also ABC-transporter(s) which share a homology (level of nucleic acid sequence) of at least 70%, preferably 80%, more preferred 90% and even more preferred above 95% identity with the above outlined ABC-transporter(s) are within the scope of the present invention, as long as these ABC-transporter(s) are able to transport hyaluronan across a lipid-bilayer. Other ABC-transporter(s), e.g. from other species or from the same species but from different sources can easily be identified, e.g. by sequence analysis using known programs like BLAST or by screening methods based on hybridizing ABC-transporter probes (nucleic acid probes for the identification of species homologues and the like). Such methods are well-known to the skilled person.

It is also envisaged that the term “ABC transporter” includes a superfamily of genes found in many organisms including humans (Higgins CF, “ABC transporters: from microorganisms to man”, Annu Rev Cell Biol 8:67-113 (1992)) characterized by a highly conserved region known as the ATP binding cassette (Hyde et al., “Structural model of ATP-binding proteins associated with cystic fibrosis, multidrug resistance and bacterial transport”, Nature 346:362-365 (1990); Mimura et al., “Structural model of the nucleotide binding conserved component of periplasmic permeases”, Proc Natl Acad Sci USA 88:84-88 (1991)). These characteristic features provide a means of identifying unknown members of the ABC transporter superfamily using various database searching techniques, e.g. BLAST (Altschul et al., “Basic local alignment search tool,” J Mol Biol 215:403-410 (1990)). Using the N-terminal ATP-binding domain of MDR1 as a conserved region of the superfamily of ABC transporters, this strategy has been utilized to identify a large family of ESTs corresponding to putative ABC transporters (Allikmets et al., “Characterization of the human ABC superfamily: isolation and mapping of 21 new genes using the Expressed Sequence Tags database”, Human Mol Genetics 5:1649-1655 (1996)). Accordingly, such new and not yet identified ABC-transporters are also within the gist of the present invention. Suitable methods for isolating/screening such new ABC-transporter(s) and for isolating homologues of such ABC-transporter(s) (i.e. homologues from other species like other mammals or the like) are well known to the skilled person (see for example; Sambrook et al, loc. cit). Such homologues as well as orthologs and analogs are for example detailed in WO9848784 which is also incorporated herein by reference.

In a preferred embodiment said human ABC-transporter(s) is(are) a member of the human ABCB (MDR)-subfamily, the ABCA subfamily and/or the human ABC-C (MRP)-subfamily.

A comprehensive recent review on ABC transporters is [143]. The web-site http://www.nutrigene.4t.com/humanabc.htm also contains valuable information. The ABC-A1 transporter is a major regulator of cellular cholesterol and phospholipid homeostasis. It mediates e.g. the efflux of phospholipids and cholesterol from macrophages to apoA-I, reversing foam cell formation. ABC-A1 deficiency observed in Tanger disease disrupts apoA-I-mediated cholesterol efflux from cell and leads to HDL deficiency and foam cell formation. It is also responsible for the intracellular transport of lipids between vesicles and plasma membrane.

The MDR1 (P-glycoprotein) transports many cytotoxic anticancer drugs and has broad substrate specificity. The substrates have few common structural features. They are usually organic molecules from 200 Da to 1900 Da. Many contain aromatic and uncharged or weakly basic groups, and are amphipathic and quite hydrophobic. MDR1 is mainly present in epithelial cells, where it localizes to the apical membrane. Typical sites are the blood-brain-, blood-testis-, blood-nerve-, and the foetal-maternal barrier. It is also abundant in the bile canalicular membrane of hepatocytes and in the apical membrane of small and large intestine.

MRP1 functions mainly as a transporter for amphipathic organic anions. It can transport hydrophobic drugs or other compounds that are conjugated or complexed to the anionic tripeptide glutathione, to glucuronic acid or to sulfate. It is found in most tissues, but relatively low in liver. Single MRP1 or double MRP1/MDR1 knockout mice are viable and fertile. The existence of double knock out mice for mdr1 and mrp1 suggested that there are compensatory mechanisms by which the loss of the transporters can be functionally offset by other transporters [144].

MRP2 deficient patients suffer from the Dubin-Johnson syndrome. MRP2 deficient rat have also been studied. From these studies it is known that MRP2 functions as a transporter for glucuronidated bilirubin in the canalicular membrane. The substrates are very similar to MRP1 including glutathione-, glucuronide-, and sulfate conjugates, but there is not a complete overlap. There is also an extensive overlap in tissue distribution between MRP2 and MDR1.

MRP3 is most closely related to MRP1 and has also very broad substrate specificity for organic anions with considerable overlap to MRP1 and MRP2, but it did not appear to be an efficient transporter for glutathione conjugates. This transporter is strongly upregulated in the liver of MRP2-deficient patients and rats.

MRP4 is strongly expressed in lung, kidney, bladder, gall bladder, tonsil, and prostate and moderately in lung, skeletal muscle, pancreas, spleen, thymus, testis, ovary, and small intestine. It can transport organic anions such as antiviral AMP analogues, cAMP, cGMP. However, it does not transport glutathione conjugates, although glutathione conjugates can inhibit the transport of cGMP. MRP4 catalysed the time- and ATP-dependent uptake of prostaglandin E1 (PGE1) and PGE2.

MRP5 analysis is still in its infancy. It is an organic anion transporter and most closely related to MRP4. MRP5 transports the fluorescent dye fluorescein diacetate [145], cAMP and cGMP and of glutathione conjugated compounds such as S-(2,4-dinitrophenyl)glutathione [146], but not leukotriene C4, 17β-glucuronosyl-estradiol, calcein, GSSG, and PGE1 or PGE2 [147]. It can be expressed in several splicing variants [148]. Knockout mice do not display obvious abnormalities. MRP5 is found in all tissues analysed thus far [141;142].

From the inhibitory profile of the drugs (see appended Example 9) and inhibitory-experiments with MRP5-specific RNAi (see Example 10) it is evident that MRP5 is the most likely hyaluronan transporter of human fibroblasts. This conclusion does not imply that MRP5 transports hyaluronan exclusively, as MRP5 knock out mice are viable thus indicating that alternative transport systems within cells can compensate for the lack of MRP5. These transporters should be ABCC11 or ABCC12 due to their close phylogenetic relationship [see FIG. 5].

In summary, the MRP1-5 transporters are known to transport organic anions and are therefore predisposed to function as hyaluronan transporter.

In a preferred embodiment the ABC-transporter of the present invention is a member of the MRP family as specified above. Preferably it is MRP5 having a sequence as deposited under Accession number NM005688, ABCC11 having a sequence as deposited under NM033151 and/or ABCC12 having a sequence as deposited under NM033226. In a most preferred embodiment it is MRP5 having a sequence as deposited under Accession number NM005688-[gi:5032100] or NM 018672-[gi:17865629].

Chondrocytes represent only 5% of the tissue. They are responsible for synthesizing and controlling the matrix. The biosynthesis of hyaluronan and proteoglycans have different mechanisms and occur in different compartments [2,3]: Proteoglycans are synthesized in the Golgi and exocytosed by vesicles. Hyaluronan is polymerized at the inner side of plasma membranes and exported by ABC-transporters. Both components aggregate in the extracellular matrix. In osteoarthritic cartilage the matrix appears more swollen and amorphous [4].

Chondrocytes alter the rates of syntheses in osteoarthritic cartilage. Hyaluronan production is increased compared to proteoglycans [5], the CD44-receptor is down regulated and enhanced synthesis of proteases and collagenases causes dissolution of collagen fibrils. Finally chondrocytes go into apoptosis. These events are triggered by cellular mediators, such as interleukin-1, and can also be observed in cell cultures. Limited interleukin-1 treatment increases proteoglycan synthesis and its concentrations returns to normal without significant over production. Thus chondrocytes appear to have a rudimentary memory. They sensor matrix loss and respond with repair. The sensors could be the CD44 receptors, because they can trigger an intracellular reaction cascade upon hyaluronan binding at the cell surface [6-10]. Osteoarthritic cartilage has lost this memory [11]. In addition to the above-described trigger via cellular mediators, almost any disturbance of the cellular homeostasis results in stimulation of hyaluronan production.

The rates of synthesis and degradation are similar in healthy cartilage. Hyaluronan can be endocytosed by CD44 and proteoglycans leave the cartilage by diffusion [12]. The well synchronized equilibrium between catabolic and anabolic processes is disturbed by cytokines and proteases. It has been demonstrated in cell cultures that interleukin-1 stimulates hyaluronan and protease synthesis and inhibits aggrecan and CD44 synthesis [9;10;13-15]. Increased hyaluronan synthesis is the primary process and occurs before the stimulation of protease synthesis [16;17]. For a long time it was thought that proteolytic degradation of collagen and aggrecan was responsible for cartilage dissolutions. However, the inefficiency of proteinase inhibitors converts this hope into an illusion [18].

It is known that the CD44 receptor transmits signals into the cell upon binding to oligosaccharides of hyaluronan- and causes increased production of metalloproteases [19]. Therefore it appears likely that an enhanced hyaluronan concentration in osteoarthritic cartilage has a similar effect and also induces protease production. But is also possible that overproduction hyaluronan is alone sufficient to desintegrate cartilage. The equilibrium between proteoglycans and hyaluronan is disturbed and the strongly swelling hyaluronan looses aggrecan decoration like a bottle brush without bristles. Permanent new synthesis of hyaluronan pushes intact proteoglycan aggregates away from chondrocytes to soften the cartilage matrix.

Therefore the primary aim of treatment must include a restriction of hyaluronan production and transport in activated chondrocytes.

Accordingly, in a further embodiment of the present invention said ABC-transporter(s) is(are) comprised in a chondrocyte cell, preferably a human chondrocyte cell. Chondrosarcoma cell lines such as CRL-7891 or HTB-94 from the American Type Culture Collection are particularly suitable for in vitro investigations on hyaluronan transport, because they produce large amounts of hyaluronan binding proteoglycan in cell culture [107-109] and constitutively express P-glycoprotein [110-112]. The respective ABC-transporter(s) which is(are) comprised in such a chondrocyte cell or cell-line can be easily detected, e.g. by FACS-analysis, Northern-Blot; Western-Blot, RT-PCR and the like. The respective nucleic acid sequences encoding the ABC-transporter(s) are described elsewhere in this specification (e.g. in FIG. 1). With regard to FACS-analysis and other protein-based assays, it is e.g. possible to employ specific antibodies which specifically detect the different ABC-transporter(s). Examples of such antibodies have been described elsewhere in this specification.

It is also known that human chondrocytes can express the ABCB1-(MDR-) gene product P-glycoprotein under certain conditions [94;95]. As mentioned before, there are several methods that allow the identification of ABC transporters in chondrocytes. First, specific antibodies can be used in histochemical immunofluorescence or in ELISA assays to detect the corresponding antigen. Second, the level of transcription can be measured by quantitative RT-PCR. Third, in situ hybridization with specific oligonucleotide cDNA probes. Fourth, microarray chip technology can be employed, wherein the expression pattern of the ABC-transporter(s) in the cell is detected via a predetermined number of given ABC-transporter-sequences on said chip. All the aforementioned methods are well-established and of course well-known to the skilled artisan.

The action of the MDR inhibitor Valspodar on hyaluronan synthesis was first investigated in a temperature sensitive human chondrocyte cell line that proliferates at 32° C. and differentiates to chondrocytes at 39° C. These chondrocytes respond to interleukin in a similar, way as chondrocytes obtained brom biopsy [73]. The chondrocytes were cultured for 5 days at 39° C. in alginate beads and then incubated in the presence of increasing concentrations of Valspodar. After 3 days the media were withdrawn and the beads were solubilized with citrate buffer. The chondrocytes were harvested by centrifugation. The concentrations of hyaluronan and proteoglycans were determined in the media, the alginate and the cells. [35S]Sulfate incorporation into proteoglycans was determined in a parallel series of experiments during the last 24 hours of incubation. The hyaluronan concentration was determined by an ELISA assay [74]. The proteoglycan concentration was measured by a colour reaction [75], and sulfate incorporation into proteoglycans was determined by radioactivity [76].

FIG. 13 shows that increasing Valspodar concentrations cause a drop in the hyaluronan concentration in interleukin induced chondrocytes and in the alginate beads to comparable levels of interleukin free cultures. The dramatic increase of hyaluronan production in alginate verifies previous results [14]. In alginate the hyaluronan does not disappear completely. This could be due to the fact that residual concentrations of hyaluronan remained in alginate from the preceding incubation that were not washed out by the media change.

FIG. 14 shows that increasing Valspodar concentrations do not cause any change in the proteoglycan synthesis rate and its concentration in alginate. The dramatic decrease of proteoglycan synthesis verifies previous results [14]. However, Valspodar causes a drop in the proteoglycan concentration on the cells. This result can be explained by the assumption that the limited hyaluronan concentration was not sufficient to retain proteoglycans.

These results show that Valspodar selectively inhibits hyaluronan synthesis in humane chondrocytes and does not influence proteoglycan synthesis. Many other ABC transporter inhibitors are acting in a similar way as Valspodar and have the same target [53]. Therefore they will exert a similar inhibitory effect on hyaluronan production. Several inhibitors have been tested to prove this hypothesis (the respective results are described herein below).

The main hyaluronan producing cells in the body are fibroblasts, sarcomas, carcinomas, smooth muscle cells, endothelial cells, endodermal cells, liver stellate cells, mesothelioma cells, melanoma cells, oligodendroglial cells, glioma cells, Schwann cells, synovial cells, myocaridal cells, trabecular-meshwork cells, cumulus cells, liver adipocytes (Ito cells), keratinocytes, epithelial cells. Thus, in a further embodiment of the present invention said ABC-transporter(s) is(are) comprised in fibroblasts, sarcomas, carcinomas, smooth muscle cells, endothelial cells, endodermal cells, liver stellate cells, mesothelioma cells, melanoma cells, oligodendroglial cells, glioma cells, Schwann cells, synovial cells, myocaridal cells, trabecular-meshwork cells, cumulus cells, liver adipocytes (Ito cells), keratinocytes, epithelial cells, preferably of human origin. The respective cell lines are available, e.g. from the American Type Culture Collection (ATCC) or from the DSMZ. Such cell lines are particularly suitable for in vitro investigations on hyaluronan transport. The respective ABC-transporter(s) which is(are) comprised in such cells or cell-lines can be easily detected, e.g. by FACS-analysis, Northern-Blot; Western-Blot, RT-PCR and the like. The respective nucleic acid sequences encoding the ABC-transporter(s) are described elsewhere in this specification (e.g. in FIG. 1). With regard to FACS-analysis and other protein-based assays, it is e.g. possible to employ specific antibodies which specifically detect the different ABC-transporter(s). Examples of such antibodies have been described elsewhere in this specification.

Accordingly, in a preferred embodiment of the present invention said inhibitor(s) is(are) selected from the group consisting of:
(a) an inhibitor of a member of the ABCB (MDR)-subfamily selected from Verapamil, Valspodar (PSC833), Elacridar (GF-120918), Bericodar (VX-710), Tariquidar (XR-9576), XR-9051, S-9788, LY-335979, MS 209, R101933; OC-144-093; Quinidine, Chloripramine, Nicardipine, Nifedipine, Amlodipine, Felodipine, Manidipine, Flunarizine, Nimodipine, Pimozide, Lomerizine, Bepridil, Amiloride, Almitrine, Amiodarone, Imipramine, Clomiphene, Tamoxifen, Toremifene, Ketocanazole, Terfenadine, Chloroquine, Mepacrin, Diltiazem, Niguldipine, Prenylamine, Gallopamil, Tiapamil, Dex-Verapamil, Dipyridamole, Pimozide, Haloperidol, Chlorpromazine, Trifluoperazine, Fluphenazine, Reserpin, Clopenthixol, Flupentixol, N-acetyldaunorubicin, Vindoline, N276-14, N276-17, B9309-068, BIBW-22, Carvedilol, Clofazimine, Ketoconazole, Lovastatin, N-Norgallopamil, Simvastatin, Troleandomycin, Vinblastin, Itraconazole, Econazole, Oligomycine, Cyclbsporin and Rapamycin; and/or
(b) an inhibitor of a member of the ABCA subfamily selected from Glyburide, DIDS (4,4-diisothiocyanatostilbene-2,2-disulfonic acid), Bumetanide, Furosemide, Sulfobromophthalein, Diphenylamine-2-carboxylic acid and Flufenamic acid; and/or
(c) an inhibitor of a member of the human ABC-C (MRP)-subfamily selected from MK-571, Benzbromaron, PAK-104P, Probenecid, Sulfinpyrazone, Indomethacin, Merthiolate and Ethacrynic acid; and/or
(d) (an) antibody(ies) or functional fragments thereof which is(are) specifically recognizing one or more ABC-transporter(s) capable of transporting hyaluronan across a lipid bilayer; and/or
(e) (an) antisense oligomere(s), iRNA and/or siRNA directed against one or more ABC-transporter(s) capable of transporting hyaluronan across a lipid bilayer; and/or
(f) (an) aptamer(s) directed against one or more ABC-transporter(s) capable of transporting hyaluronan across a lipid bilayer.

The meaning and scope of the terms N276-14, N276-17, B9309-068, is well-described in the following references, which are therefore incorporated herein by reference. The compound B9309-068 (3,4-dihydro-2,7,dimethyl-3-[2-(4-morpholinyl)ethyl]-5-(3-nitrophenyl)-4-oxo-6-[1-oxo-1]-(4,4-diphenyl-1-piperidinyl)undecyl)-p-yrido[2,3-d]pyrimidine-dihydrochloride) is described on pages 157-168 of Beck, J. F., Buchholz, F., Ulrich, W. R., Boer, R., Sanders, K. H., Niethammer, D., & Gekeler, V. (1998) Rhodamine 123 efflux modulation in the presence of low or high serum from CD56+hematopoietic cells or CD34+leukemic blasts by B9309-068, a newly designed pyridine derivative. Cancer Lett., 129, 157-163. The compounds terms N276-14, N276-17 are described on pages 123-132 of Naito, S., Koike, K., Ono, M., Machida, T., Tasaka, S., Kiue, A., Koga, H., & Kumazawa, J. Development of novel reversal agents, imidazothiazole derivatives, targeting MDR1- and MRP-mediated multidrug resistance. Oncol. Res. 10, 123-132. 1996.

The term/compound LY-335979 is for example described in (Dantzig et al., (1996) Cancer Research 56:4171-4179) as well as in WO9917757.

Further references for some specific inhibitors which are also within the scope of the present invention are exemplified below:

XR9576: Martin C; Berridge G; Mistry P; Higgins C; Charlton P; Callaghan R The molecular interaction of the high affinity reversal agent XR9576 with P-glycoprotein. BRITISH JOURNAL OF PHARMACOLOGY (1999 September), 128(2), 403-11.

Ryder, Hamish; Ashworth, Philip Anthony; Roe, Michael John; Brumwell, Julie Elizabeth; Hunjan, Sukhjit; Folkes, Adrian John; Sanderson, Jason Terry; Williams, Susannah; Maximen, Levi Michael; et al. Anthranilic acid derivatives as multi drug resistance modulators. PCT Int. Appl. (1998), 203 pp. CODEN: PIXXD2 WO 9817648 A1 19980430 CAN 128:321568 AN 1998:268489

R101933: Van Zuylen, Lia; Sparreboom, Alex; Van der Gaast, Ate; Van der Burg, Maria E. L.; Van Beurden, Vera; Bol, Cornelis J.; Woestenborghs, Robert; Palmer, Peter A.; Verweij, Jaap. The orally administered P-glycoprotein inhibitor R101933 does not alter the plasma pharmacokinetics of docetaxel. Clinical Cancer Research (2000), 6(4), 1365-1371

OC-144-093: Mjalli, Adnan M. M.; Zhang, Chengzhi. Preparation of imidazole derivatives as MDR modulators. U.S. (1998), 56 pp., Cont.-in-part of U.S. Ser. No. 890,911, abandoned. CODEN: USXXAM U.S. Pat. No. 5,840,721 A 19981124 CAN 130:25072 AN 1998:774230

Newman, Michael J.; Rodarte, Jennifer C.; Benbatoul, Khalid D.; Romano, Suzanne J.; Zhang, Chengzhi; Krane, Sonja; Moran, Edmund J.; Uyeda, Roy T.; Dixon, Ross; Guns, Emma S.; Mayer, Lawrence D. Discovery and characterization of OC144-093, a novel inhibitor of P-glycoprotein-mediated multidrug resistance. Cancer Research (2000), 60(11), 2964-2972

Quinidine: Rudner, Bernard. Quinuclidine derivatives. (1959), US 2892832 19590630 CAN 54:7355 AN 1960:7355

Chloripramine: Protiva, M.; Hnevsova-Seidlova, V.; Jirkovsky, U.; Novak, L.; Vejdelek, Z. J. Synthetic ataractics. Ill. 2′-Substituted 2,3:6,7-dibenzosuberans with 3-dimethylaminopropane residue in 1-position. Cesko-Slovenska Farmacie (1962), 10M 506-15.

Villani, Frank J. 5-(3-Dimethylamino-2-methylpropyl)dibenzocycloheptenes. U.S. (1968), 10 pp. CODEN: USXXAM U.S. Pat. No. 3,409,640 19681105 CAN 70:68009 AN 1969:68009

Nicardipine: Iwanami, Masaru; Murakami, Masuo; Takahashi, Kozo; Fujimoto, Masaharu; Shibanuma, Tadao; Kawai, Ryutaro; Taken'aka, Toichi. 1,4-Dihydropyridine-3,5-dicarboxylic acid aminoalkyl ester derivatives. Jpn. Kokai Tokkyo Koho (1974), 15 pp. CODEN: JKXXAF JP 49109384 19741017 Showa. CAN 82:170692 AN 1975:170692

Nifedipine: Bossert, Friedrich; Vater, Wulf. 4-Aryl-1,4-dihydropyridine. S. African (1968), 25 pp. CODEN: SFXXAB ZA 6801482 19680807 CAN 70:96641 AN 1969:96641

Amlodipine: Campbell, Simon Fraser; Cross, Peter Edward; Stubbs, John Kendrick. Dihydropyridine anti-ischemic and antihypertensive agents and pharmaceutical compositions containing them. Eur. Pat. Appl. (1983), 55 pp. CODEN: EPXXDW EP 89167 A2 19830921 CAN 100:6351 AN 1984:6351

Felodipine: Berntsson, Peter Bernhard; Carlsson, Stig Ake Ingemar; Gaarder, Jan Ornulf; Ljung, Bengt Richard. 2,6-Dimethyl-4,2,3-disubstituted phenyl-1,4-dihydropyridine-3,5-dicarboxylic acid-3,5-asymmetric diesters having hypotensive properties. Eur. Pat. Appl. (1980), 44 pp. CODEN: EPXXDW EP 7293 19800123 CAN 93:26283 AN 1980:426283

Manidipine: Meguro, Kanji; Nagaoka, Akinobu. Dihydropyridine derivatives. PCT Int. Appl. (1983), 44 pp. CODEN: PIXXD2 WO 8304023 A119831124 CAN 100:120900 AN 1984:120900

Flunarizine: Ritzmann, Goetz; Etschenberg, Eugen; Ghyczy, Miklos. Parenterally applicable aqueous pharmaceutical solutions of diphenylmethylpiperazine derivatives. Ger. Offen. (1981), 5 pp. CODEN: GWXXBX DE 2949707 19810619 CAN 95:68059 AN 1981:468059

Nimodipine: Meyer, Horst; Bossert, Friedrich; Kazda, Stanislav; Hoffmeister, Friedrich. Cerebral therapeutic agent: 1,4-dihydro-2,6-dimethyl-4-(3′-nitrophenyl)-pyridine-3-(□-methoxyethyl ester)-5-(isopropyl ester). Brit. UK Pat. Appl. (1979), 7 pp. CODEN: BAXXDU GB 2018134 19791017 CAN 92:220691 AN 1980:220691

Pimozide: Janssen, C. Benzimidazolinylpiperidines and -tetrahydropyridines. (1963), 27 pp. BE 633495 19631211 CAN61:11373 AN 1964:411373

Lomerizine: Ohtaka, Hiroshi; Kanazawa, Toshiro; Ito, Keizo; Tsukamoto, Goro. piperazine derivatives, and pharmaceutical compositions comprising these compounds as active ingredients. Eur. Pat. Appl. (1985), 32 pp. CODEN: EPXXDW EP 159566 A1 19851030 CAN 104:155975 AN 1986:155975

Bepridil: Mauvernay, Roland Yves; Busch, Norbert; Simond, Jacques; Monteil, Andre; Moleyre, Jacques. 3-Isobutoxy-2-pyrrolidino-N-phenyl-N-benzylpropylamine. Ger. OfFen. (1978), 12 pp. CODEN: GWXXBX DE 2802864 19780727 CAN 89:179852 AN 1978:579852

Amiloride: Cragoe, Edward J., Jr. Substituted guanidines. (1964), 99 pp. BE 639386 19640430 CAN 62:82636 AN 1965:82636

Almitrine: de Castro J; Andrieu S; Clerckx A The use of almitrine following potentialized analgesic. Anesthesia based upon alfentanil and flunitrazepam or etomidate. ACTA ANAESTHESIOLOGICA BELGICA (1979), 30 Suppl 135-49.

Amiodarone: Grain, Claude; Jammot, Fernand. Aluminum chloride addition compounds with benzoylbenzofurans. Fr. Demande (1986), 27 pp. CODEN: FRXXBL FR 2583754 A1 19861226 CAN 108:112215 AN 1988:112215

Imipramine: Haefliger, Franz; Schindler, Walter. Tertiary-aminoalkyliminodibenzyls. (1951), US 2554736 19510529 CAN46:17775 AN 1952:17775

Clomiphene: Allen, Robert E.; Palopoli, Frank P.; Schumann, Edward L.; Van Campen, Marcus G., Jr. 1-[p-(□-Diethylaminoethoxy)phenyl]-1,2-diphenyl-2-chloroethylene. (1963), 3 pp. DE 1155436 19631010 CAN 60:52528 AN 1964:52528

Tamoxifen: Harper, Michael J. K.; Richardson, Dora Nellie; Walpole, Arthur L. Substituted-aminoalkoxyphenylalkene isomer separations. Brit. (1967), 7 pp. CODEN: BRAAAA GB 1064629 19670405 CAN 67:90515 AN 1967:490515

Toremifene: Ioivola, Reijo Juhani; Karjalainen, Ario Johannes; Kurkela, Kauko Oiva Antero; Soderwall, Marja Liisa; Kangas, Lauri Veikko Matti; Blanco, Guillermo Luis; Sunduiqst, Hannu Kalervo. Triphenylalkane and -alkene derivatives and their use. Eur. Pat. Appl. (1983), 82 pp. CODEN: EPXXDW EP 95875 A2 19831207 CAN 101:6807 AN 1984:406807

Ketocanazole: Antimicrob Agents Chemother. 1980 June; 17(6):922-8. In vitro and in vivo effects of the antimycotic drug ketoconazole on sterol synthesis. Van den Bossche H, Willemsens G, Cools W, Cornelissen F, Lauwers W F, van Cutsem J M.

Terfenadine: Carr, Albert Anthony; Kinsolving, Clyde R. Antihistaminic and broncholytic piperidinoalkanols. Ger. Offen. (1973), 25 pp. CODEN: GWXXBX DE 2303306 19730802 CAN 79:105083 AN 1973:505083

Chloroquine: Yoshida, Shin-ichiro. 7-Chloro-4-(4-diethylamino-1-methylbutylamino)quinoline. (1949), JP 179272 19490606 CAN 46:8743 AN 1952:8743

Mepacrine: Cowdrey, Wm. A.; Murray, Arthur G. Mepacrine dihydrochloride. (1946), US 2410406 19461105 CAN 41:10014 AN 1947:10014

Diltiazem: Pharmaceuticals containing histaminic H2 receptor antagonists. Jpn. Kokai Tokkyo Koho (1980), 3 pp. CODEN: JKXXAF JP 55043036 19800326 Showa. CAN 93:173742 AN 1980:573742

Niguldipine: Ulrich, Wolf Ruediger; Amschler, Hermann; Flockerzi, Dieter; Klemm, Kurt; Kohl, Bernhard; Eistetter, Klaus; Eltze, Manfrid; Kolassa, Norbert; Sanders, Karl; Schudt, Christian. Preparation of 1,4-dihydropyridine-3,5-dicarboxylate enantiomers as antihypertensives and cardiovascular agents. PCT Int. Appl. (1988), 62 pp. CODEN: PIXXD2 WO 8807525 A119881006 CAN 110:173094 AN 1989:173094

Prenylamine: Ehrhart, Gustav; Lindner, Ernst; Ott, Heinrich. Diphenylmethane derivs. with basic substituents. CAN 56:45811 AN 1962:45811

Gallopamil: Einig, Heinz; Buehler, Volker; Koch, Winfried; Schwarz, Joachim Arthur. Sustained-release oral pharmaceuticals. Ger. Offen. (1982), 11 pp. CODEN: GWXXBX DE 3113901 A1 19821028 CAN98:59901 AN 1983:59901

Tiapamil: Ramuz, Henri. Dithiane derivatives. Ger. Offen. (1975), 68 pp. CODEN: GWXXBX DE 2460593 19750703 CAN 83:179076 AN 1975:579076

Dex-Verapamil: Treiber, Hans Joerg; Raschack, Manfred; Dengel, Ferdinand. d-2-Isopropyl-5-(N-methyl-N-phenethylamino)-2-phenylvaleronitriles. Ger. Offen. (1972), 27 pp. CODEN: GWXXBX DE 2059985 19720615 CAN 77:101248 AN 1972:501248

Dipyridamole: Derivatives of pyrimido[5,4-d]pyrimidine. (1959), GB 807826 19590121 CAN 53:67806 AN 1959:67806

Pimozide: Janssen, C. Benzimidazolinylpiperidines and -tetrahydropyridines. (1963), 27 pp. BE 633495 19631211 CAN61:11373 AN 1964:411373

Haloperidol: Janssen, P. A. J. 1-Aroylalkyl-4-arylpiperidin-4-ol derivatives. (1959), BE 577977 19590515 CAN 54:23229 AN 1960:23229

Chlorpromazine: Charpentier, Paul. 10-Dialkylaminoalkyl-2- or 4-chlorophenothiazines. (1953), US 2645640 19530714 CAN 49:16278 AN 1955:16278

Trifluoperazine: 10-(Aminoalkyl)trifluoromethylphenothiazine derivatives. (1959), GB 813861 19590527 CAN 54:7371 AN 1960:7371

Fluphenazine: Perfluoroalkylphenothiazines. (1960), GB 829246 19600302 CAN 54:91813 AN 1960:91813

Reserpin Reserpine derivatives and the manufacture of reserpine and derivatives. (1960), GB 846482 19600831 CAN 55:38153 AN 1961:38153

Clopenthixol: Lassen, Niels. 2-Chloro-9-[3-[N-(2-hydroxyethyl)piperazino]propylidene]thioxanthene □-isomers, its esters and salts for pharmaceutical preparations. Ger. Offen. (1975), 25 pp. CODEN: GWXXBX DE 2429101 19750130 CAN 82:156372 AN 1975:156372

Flupentixol: Trifluoromethylxanthene and -thiaxanthene derivatives. (1963), 9 pp. GB 925538 19630508 CAN 59:69194 AN 1963:469194

N-acetyldaunorubicin: Skovsgaard T Circumvention of resistance to daunorubicin by N-acetyldaunorubicin in Ehrlich ascites tumor. CANCER RESEARCH (1980 April), 40(4), 1077-83.

Vindoline: Kutney, James P. Synthesis of vinblastine, vincristine, and related compounds. U.S. Pat. Appl. (1975), 19 pp. Avail. NTIS. CODEN: XAXXAV US 582373 19750530 CAN 85:160409 AN 1976:560409

Carvedilol: Wiedemann, Fritz; Kampe, Wolfgang; Thiel, Max; Sponer, Gisbert; Roesch, Egon; Dietmann, Karl. Carbazolyl-4-oxypropanolamine derivatives. Ger. Offen. (1979), 27 pp. CODEN: GWXXBX DE 2815926 19791018 CAN 92:128716 AN 1980:128716

Clofazimine: Medlen, Constance Elizabeth; Anderson, Ronald; O'Sullivan, John Francis Gladore. Use of a riminophenazine for treating MDR resistance. Eur. Pat. Appl. (1995), 27 pp. CODEN: EPXXDW EP 676201 A2 19951011 CAN 124:250909 AN 1996:200111

Ketoconazole: Heeres, Jan; Backx, Leo J. J.; Mostmans, Joseph H. Fungicidal and bactericidal 1-(1,3-dioxolan-2-ylmethyl)-1H-imidazoles and -1H-1,2,4-triazoles and their salts. Ger. Offen. (1978), 72 pp. CODEN: GWXXBX DE 2804096 19780803 CAN 89:180014 AN 1978:580014

Lovastatin: Endo, Akira. Monacolin K and pharmaceutical composition containing it. Ger. Offen. (1980), 17 pp. CODEN: GWXXBX DE 3006216 19800904 CAN 93:184283 AN 1980:584283

N-Norgallopamil: Mutlib A E; Nelson W L Synthesis and identification of the N-glucuronides of norgallopamil and norverapamil, unusual metabolites of gallopamil and verapamil. JOURNAL OF PHARMACOLOGY AND EXPERIMENTAL THERAPEUTICS (1990 February), 252(2), 593-9.

Simvastatin: Willard, Alvin K.; Smith, Robert L.; Hoffman, William F. 6(R)-[2-(8-acyloxy-2-methyl-6-methyl (or hydrogen)-polyhydro-1-naphthyl)ethyl]-4(R)-hydroxy-3,4,5,6-tetrahydro-2H-pyran-2-ones, the hydroxy acid forms of these pyranones, salts and esters thereof, and a pharmaceutical antihypercholesterolemic composition containing them. Eur. Pat. Appl. (1981), 54 pp. CODEN: EPXXDW EP 33538 19810812 CAN 95:219968 AN 1981:619968

Troleandomycin: Rodionovskaya, E. I.; Trakhtenberg, D. M.; Kleiner, G. I. Triacetyloleandomycin. U.S.S.R. (1969), CODEN: URXXAF SU 224754 19690212 CAN 71:39394 AN 1969:439394

Vinblastin: Vincaleukoblastine. (1961), GB 870723 19610621 CAN 55:127250 AN 1961:127250

Itraconazole: Gallardo Carrera, Antonio. 1-Phenacyl-1,2,4-triazole ketals. Span. (1985), 7 pp. CODEN: SPXXAD ES 539139 A119851116 CAN 106:67318 AN 1987:67318

Econazole:Godefroi, Erik F.; Heeres, Jan. 1-(□-Arylethyl)imidazoles with fungistatic and bactericidal effects. Ger. Offen. (1970), 71 pp. CODEN: GWXXBX DE 1940388 19700226 CAN 72:90466 AN 1970:90466

Oligomycine: Mechetner, Eugene; Roninson, Igor B. Methods and reagents for preparing and using immunological agents specific for P-glycoprotein. U.S. (1999), 56 pp., Cont.-in-part of U.S. Pat. No. 5,891,654. CODEN: USXXAM U.S. Pat. No. 5,994,088 A 19991130 CAN 132:10503 AN 1999:761466

Rapamycin: Sehgal, Surendra N.; Blazekovic, Teodora M.; Vezina, Claude. Rapamycin, a new antibiotic. Ger. Offen. (1974), 24 pp. CODEN: GWXXBX DE 2347682 19740411 CAN 81:24166 AN 1974:424166

Glyburide: Method for the preparation of sulfonylureas and sulfonylsemicarbazides. Neth. Appl. (1966), 11 pp. CODEN: NAXXAN NL 6603398 19660919 CAN 66:65289 AN 1967:65289.

DIDS (4,4-diisothiocyanatostilbene-2,2-disulfonic acid): Cardin, Alan D.; Tyms, Stanley A. Sulfonic stilbene derivatives in the treatment of viral diseases. Eur. Pat. Appl. (1992), 22 pp. CODEN: EPXXDW EP 498095 A119920812 CAN 117:208840 AN 1992:608840

Bumetanide: Feit, Peter W. Antiedematous and antihypertensive 3-butylamino-4-phenoxy-5-sulfamoylbenzoic acid. Ger. Offen. (1970), 32 pp. CODEN: GWXXBX DE 1964504 19700709 CAN 73:55831 AN 1970:455831

Furosemide: Sturm, Karl; Siedel, Walter; Weyer, Rudi. Sulfamoylanthranilic acids. (1962), DE 1122541 19620125 CAN56:73016 AN 1962:73016

Sulfobromophthalein: Miyatake, Kazuo; Suzuki, Keiichi. Sodium tetrabromophenolphthaleindisulfonate. (1951), JP 26005341 19510915 Showa. CAN 47:55042 AN 1953:55042

Diphenylamine-2-carboxylic acid: Goldberg, Alan A.; Kelly, Wm. Diphenylaminecarboxylic acid derivatives. (1948), GB 600640 19480414 CAN 42:34402 AN 1948:34402

Flufenamic acid: N-(3-Trifluoromethylphenyl)anthranilic acid antiinflammatory drug. (1962), 9 pp. FR M1341 19620702 CAN 58:59558 AN 1963:59558

MK-571: Young, Robert N.; Zamboni, Robert; Leger, Serge. Preparation of 2-substituted quinoline dioic acids as leukotriene antagonists and inhibitors of their biosynthesis. Eur. Pat. Appl. (1987), 44 pp. CODEN: EPXXDW EP 233763 A2 19870826 CAN 109:37743 AN 1988:437743

Benzbromaron:Substituted coumaranones. (1957), BE 553621 19570621 CAN 53:122289 AN 1959:122289.

PAK-104P: Shudo, Norimasa; Mizoguchil, Tetsuro; Kiyosue, Tatsuto; Arita, Makoto; Yoshimura, Akihiko; Seto, Kiyotomo; Sakoda, Ryozo; Akiyama, Shinichi. Two pyridine analogs with more effective ability to reverse multidrug resistance and with lower calcium channel blocking activity than their dihydropyridine counterparts. Cancer Research (1990), 50(10), 3055-61

Probenecid: Sulfamylbenzoic acids. (1952), GB 674298 19520618 CAN 47:28834 AN 1953:28834.

Sulfinpyrazone: Klemm, Kurt; Langenscheid, Erhard. 1,2-Diphenylpyrazolidine-3,5-diones. Ger. Offen. (1970), 18 pp. CODEN: GWXXBX DE 1814649 19700625 CAN 73:45507 AN 1970:445507

Indomethacin: Winter, Charles A. Antiinflammatory compositions containing indole derivatives. (1963), 40 pp. BE 621313 19630211 CAN 59:62164 AN 1963:462164

Merthiolate: Uckun, Fatih M. Preparation and pharmaceutical use of 4-(4′-hydroxyphenyl)amino-6,7-dimethoxyquinazoline to prevent the development of colorectal cancer. PCT nt. Appl. (2001), 19 pp. CODEN: PIXXD2 WO 0136394 A1 20010525 CAN 134:366897 AN 2001:380561

Ethacrynic acid: Schultz, Everett M.; Sprague, James M. 4-(□-Alkylideneacyl)-3-halophenoxyacetic acids. (1962), 162 pp. BE 612755 19620717 CAN 59:68942 AN 1963:468942

Pharmacologic profiles for several of these drugs and preferred drugs to be used in accordance with the invention are specified further in Example 9.

As mentioned before, several inhibitors of ABC-transporter(s) have already been used in the past in the context of a reduction of the so-called multi-drug-resistance (MDR) in tumors. The known multidrug resistance inhibitors have particularly been designed and optimized to block the export of chemostatic drugs in human tumours by ABC transporters. But these drugs are not the physiological substrates for the transporters. Our work demonstrated for the first time that hyaluronan is a physiological substrate and that its production can be inhibited by the known inhibitors. It is therefore a great advantage that these inhibitors and the knowledge that has accumulated with the use of multidrug resistance inhibitors can now be utilized to inhibit hyaluronan production for the treatment of human diseases (as described herein). Accordingly, it is envisaged that the use of such MDR inhibitors is encompassed by the present invention (this also relates to such inhibitors which are not mentioned expressis verbis herein—however, the skilled artisan has access to such ABC-transporter inhibitors, e.g. by searching the internet, review articles, textbooks and the like which relate to and/or disclose ABC-transporter inhibitors). Reference may be made to the literature for sources of further appropriate ABC-transporter inhibitors to use according to the invention. Thus, it has to be understood that the inhibitor(s) of the invention are not limited to the specific inhibitor(s) mentioned herein. The following inhibitor(s) merely represent an preferred selection of suitable inhibitor(s) within the meaning of the present invention.

Furthermore, it has to be understood that the specific inhibitor(s) specified for example herein, can be further modified to achieve (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmakinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophylic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetales, or (xiv) N-acetylation to amides, phenylcarbamates, or (xv) synthesis of Mannich bases, imines, or (xvi) transformation of ketones or aldehydes to Schiff's bases, oximes, acetales, ketales, enolesters, oxazolidines, thiozolidines or combinations thereof.

Several MDR modulators of the third generation have recently been developed using structure-activity relationships and combinatorial chemistry approaches targeted against specific MDR mechanisms. These agents exhibit effective inhibiting concentrations in the nanomolar range. These agents are better tolerated.

In a preferred embodiment said inhibitor is selected from the group consisting of Verapamil, Vaispodar (PSC833), Elacridar (GF-120918), Bericodar (VX-710), Tariquidar (XR-9576), XR-9051, S-9788, LY-335979, MS 209, Laniquidar (R101933); OC-144-093; BIBW-22 Zaprinast® and Lysodren®. Said compounds are, for example, described in U.S. Pat. No. 3,261,859 (Verapamil); in WO 94/07858 (Bericodar); in U.S. Pat. No. 5,405,843 or EP 0 363 212 B1 (MS 209); in EP-B1 0 296 122 (Valspodar (PSC833)), in WO 98/17648 (Tariquidar (XR-9576)); in EP-B1 0 466 586 (S-9788); in U.S. Pat. No. 5,756,527 (OC-144-093); in EP-A1 0 494 623 (Elacridar (GF-120918)); DE 4225353 (BIBW-22). Zaprinast® and Lysodren® are well-known brand names.

The following compounds illustrate inhibitors which are to be used as inhibitors of the present invention.

Verapamil; U.S. Pat. No. 3,261,859:

1. The compound of the formula

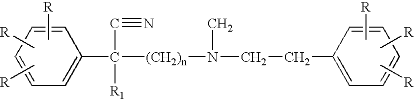

in which
R₁ is a member selected from the group consisting of lower alkyl, cyclohexyl, and phenyl,
R is hydrogen and a least one of the following; chlorine, lower alkoxy, lower alkyl, and n is on integer from 2 to 3, inclusive,
   or its pharmaceutically acceptable acid addition salts.

Further derivatives as well as methods for producing said compound are detailed in U.S. Pat. No. 3,261,859 which is, therefore, included herein in its entirety by reference.

### Laniquidar (R101 933);

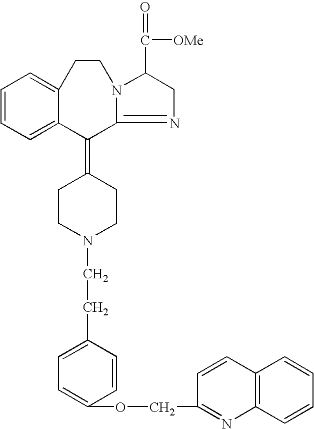

C₃₇H₃₆N₄O₃. Methyl 6,11-dihydro-11-[1-[2-[4-(−2-quinolylmethoxy)phenyl]ethyl]-4-piperidinylidene]-5H-imida-zo[2,1-b][3]benzazepine-3-carboxylate. CA 278798-78-0

### Bericodar (VX-710); WO 94/07858

1. A compound of formula (I):

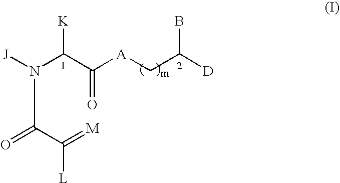

wherein A is CH₂, oxygen, NH or N—(C1-C4 alkyl);
wherein B and D are independently
(i) Ar, (C1-C10C)-straight or branched alkyl, (C2-C10C)-straight or branched alkenyl or alkynyl, (C5-C7)-cycloalkyl substituted (C1-C6)-straight or branched alkyl (C2-C6)-straight or branched alkenyl or alkynyl, (C5-C7)-cycloalkenyl substituted (C1-C6)-straight or branched alkyl, (C2-C6)-Straight or branched alkenyl or alkynyl, or Ar substituted (C1-C8)-straight or branched alkyl, (C2-C₆)-straight or branched alkenyl or alkynyl wherein, in each case, any one of the CH₂ groups of said alkyl, alkenyl or alkynyl chains may be optionally replaced by a heteroatom selected from the group consisting of O, S, SO, SO₂, N, and NR, wherein R is selected from the group consisting of hydrogen (C1-C4)-straight or branched alkyl, (C2-C4)-straight or branched alkenyl or alkynyl, and (C1-C4) bridging alkyl wherein a bridge is formed between the nitrogen and a carbon atom of said heteroatom-containing chain to form a ring, and wherein said ring is optionally fused to an Ar group; or

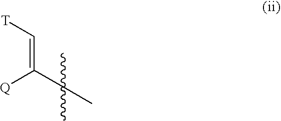

wherein Q is hydrogen, (C1-C6)-straight or branched alkyl or (C2-C6)-straight or branched alkenyl or alkynyl;

wherein T is Ar or substituted 5-7 membered cycloalkyl with substituents at positions 3 and 4 which are independently selected from the group consisting of oxo, hydrogen, hydroxyl, O—(C1-C4)-1- or O—(C2-C4)-alkenyl;

provided that at least one of B or D is independently selected from the group consisting of (C2-C10)-straight or branched alkynyl, (C2-C7)-cycloalkyl substituted (C2-C6)-straight or branched alkynyl, (C5-C7)-cycloakenyl substituted (C2-C6)-straight or branched alynyl, and Ar substituted (C2-C₆)-straight or branched alkynyl;

wherein Ar is a carbocylic aromatic group selected from the group consisting of phenyl, 1-naphthyl, 2-naphthyl, indenyl, azulenyl, fluorenyl, and anthracenyl; or a

heterocyclic aromatic group selected front the group consisting of 2-furyl, 3-furyl, 2-thienyl, 3-thienyl, 2-pyridyl, 3-pyridyl, 4-pyridyl, pyrrolyl oxazolyl, thiazolyl, imidazolyl, pyrazolyl, 2-pyrazolinyl, oxadiazolyl, 1,2,3-triazolyl 1,3,4-thiadiazolyl, pyridazinyl, pyrimidinyl, pyrazinyl, 1,3,5-triazinyl, 1,3,5-trithianyl Indolizinyl, indolyl, isoindolyl, 3H-indolyl, indolnyl, benzo[b]furanyl, benzo[b]thiophenyl, 1H-indazolyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-napthyridinyl, pteridinyl, carbazolyl, acridinyl, phenazinyl, phenothiazinyl, and phenoxazinyl;

wherein Ar may contain one to three substituents which are independently selected from the group consisting of hydrogen, halogen, hydroxyl, nitro, trifluoromethyl, trifluoromethoxy, (C1-C6)-straight or branched alkyl (C2-C6)-straight or branched alkyl, O—(C1-C4)-straight or branched alkyl, O—(C2-C₄)-straight or branched alkenyl, O-benzyl, O-phenyl, 1,2-methylenedioxy, amino, carboxyl, N-(C1-C5-straight or branched alkyl or alkenyl) carboxamides, N,N-di-(C1-C5)-straight or branched alkyl or C2-C5-straight or branched alkenyl) carboxamides, N-morpholinocarboxamide, N-benzylcarboxamide O—X, CH₂—(CH₂)_{q}—X, O—(CH₂)_{q}—X, (CH₂)_{q}—O—X, and CH═CH—X

wherein X is 4-methoxyphenyl 2-pyridyl, 3-pyridyl, 4-pyridyl, pyrazyl, quinolyl 3,5-dimethylisoxazoyl, isoxazoyl, 2-methylthiazoyl, thiazoyl, 2-thienyl, 3-thienyl, and pyrimidyl, and q is 0-2;

wherein L is either hydrogen or U; M is either oxygen or CF-U, provided that if L is hydrogen, then M is CH-U or if M is oxygen, then L is U;
wherein U is hydrogen, O-(C1-C4)-straight or branched alkyl or O—(C2-C₄)straight or branched alkenyl, (C1-C6)-straight or branched alkyl or (C2-C6)-straight or branched alkenyl, (C₅-C₇)-cycloalkyl or (C5-C7)-cycloalkenyl substituted with (C1-C4)-straight or branched alkyl or (C2-C₄)-straight or branched alkenyl, [(C1-C4)-alkyl or (C2-C₄)-alkenyl]-Y or Y;

wherein Y is a carbocyclic aromatic group selected from the group consisting of phenyl, 1-naphthyl, 2-naphthyl indenyl, azulenyl, fluorenyl, and anthracenyl; or a

heterocyclic aromatic groups as defined above;

wherein Y may contain one to three substituents which are independently selected from the group consisting of hydrogen, halogen, hydroxyl, nitro, trifluoromethyl, trifluoromethoxy, C1-C6)-straight or branched alkyl, (C1-C6) straight or branched alkenyl, O—(C1-C4)-straight or branched alkenyl, O—(C2-C₄)-straight or branched alkenyl, O-benzyl, O-phenyl, 1,2-methylenedioxy, amino, and carboxyl;

wherein J is hydrogen, (C1-C2) alkyl or benzyl; K is (C1-C4)-Straight or branched alkyl, benzyl or cyclohexylmethyl, or wherein J and K may be taken together to for a 5-7 membered heterocyclic ring which may contain a heteroatom selected from the group consisting of O, S, SO and SO₂; and

wherein m is 0-3.

Further derivatives as well as methods for producing said compound are detailed in WO 94/07858 which is, therefore, included herein in its entirety by reference.

XR-9051 (3-[(3Z, 6Z)-6 Benzylidene-1-methyl-2,5-dioxopiperazin-3-ylidenemethyl]-N-[4-[2-(6,7-dimethoxy-1,2,3,4-tetrahydroisoquinolin-2-yl)ethyl]phenyl]benzamide hydrochloride, [AC # 57-22-7]).

### Valspodar; EP-B1 0 296 122:

A compound (i) of formula II

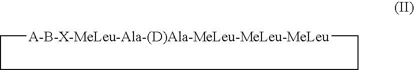

wherein
A is -3′-O-acetyl-MeBmt-
B is -αAbu-, -Thr-, -Val- or Nva-; and

when
B is -αAbu-,
X is -(D)Ala- and Y is -Val-;

when
B is -Thr- or -Val-,
X is -Sar- and Y is -Val-; or

when
B is -Nva-,
X is -Sar- and Y is -Val-, or
X is -(D)Ala- and Y is -Val-; or

wherein
A is -3′-O-acetyl-dihyrod-MeBmt or -cis-MeBmt-,
B is -αAbu-, X, is -Sar- and Y is -Val-; or

ii) of formula II′

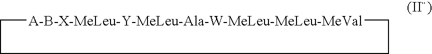

wherein
A is -3′-O-acyl-MeBmt- or -3′-O-acyl-dihydro-MeBmt-residue,
B is -αAbu-, -Thr-, -Val-, -Nva-, or the residue of a β-O-acyl-α-amino acid,
X is -Sar- or the residue of an optically active α-N-methylated α-amino acid residue having the (D)-configuration,
Y is -Val- or additionally, when B is -Nva-, -Nva-, and
W is the residue of a β-hydroxy- or β-O-acyl-α-amino acid having the (D) configuration, or

iii) wherein the residue at the position 1-position is an -8′-C₁₋₈alkoxy-cis-MeBmt- or -dihydro-MeBmt- or -3′-O-acyl-B′—C₁₋₈alkoxy-cis-MeBmt- or dihyrdo-MeBmt-residue; a -3′-O-acyl-cis-MeBmt-residue; a -7′-desmethyl-7′-hydrocarbyl- -MeBmt- or -cis-MeBmt or -3′-O-acyl-7′-desmethyl-7′-hydrocarbyl- -MeBmt- or cis-MeBmt-reside wherein a hydrocarbyl moiety comprises at least two carbon atoms; or a -7′-desmethyl-7′-hydrocarbyl-dihydro-MeBmt- or -3′-O-acetyl-7′-desmethyl-7′-hydrocarbyl-dihydro-MeBmt-residue wherein the hydrocarbyl moiety comprises at least two carbon atoms and wherein any aliphatic group or moiety as or comprising said hydrocarbyl moiety is saturated; or

(iv) wherein the 3′-carbon atom of the residue at the 1-position is oxo, C₁₋₄alkoxyamino, azidoalkylcarbonyloxy or alkoxycarbonyloxy substituted, or wherein the β-carbon atom of the residue at the 2-position is β-oxo substituted or the residue at the 2-position is an (L)-isoleucyl residue; or

(v) of formula XI

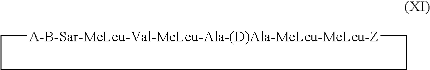

wherein
A is -N-desmethyl-dihydro-MeBmt-, B is -Thr- and Z is -MeVal-, or
A is -dihydro-MeBmt-, B is Thr- and Z is -Val-, or
A is -MeLeu-, B is -aAbu- and Z is -Val-; or which is

(vi) a dicarboxylic acid di-ester of a cyclosporin having a β-hydroxy-(L)-α-amino acid residua at the 2-position.

Further derivatives as well as methods for producing said compound are detailed in EP-B1 0 296 122 which is, therefore, included herein in its entirety by reference.

### Tariquidar (XR-9576); WO 98/17648

1. A compound which is an anthranilic acid derivative of formula (I):

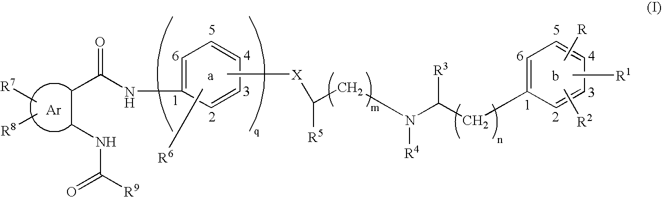

wherein
each of R, R¹ and R² which are the same or different, is H, C₁-C₆ alkyl, OH, C₂-C₆ alkoxy, halogen, nitro, or N(R¹⁰R¹¹) wherein each of R¹⁰ and R¹¹ which are the same or different, is H or C₁-C₆ alkyl, or R¹ and R², being attached to adjacent positions of ring b, together form a methylenedioxy or ethylenedioxy group;
R³ is H or C₁-C₆ alkyl
R⁴ is C₁-C₆ alkyl or R⁴ represents —CH₂— or —CH₂CH₂— which is attached either (i) to position 2 of ring b to complete a saturated 5- or 6-membered nitrogen-containing ring fused to ring b, or (ii) to the position in ring a adjacent to that to which X, being a single bond, is linked, thereby completing a saturated 5- or 6-membered nitrogen-containing ring fused to ring a;
R⁵ is H, OH or C₁-C₆ alkyl;
X is a direct bond O, S, —S—(CH₂)ₚ— or —O— (CH₂)ₚ— wherein p is an integer of 1 to 6;
R₆ is H, C₁-C₆ alkyl or C₁-C₆ alkoxy;
q is 0 or 1;
Ar is an unsaturated carbocyclic or heterocyclic group;
each of R⁷ and R⁸, which are the same or different, is II, C₁-C₆ alkyl which is unsubstituted or substituted, C₁-C₆ alkoxy, hydroxy, halogen, phenyl, —NHOH, nitro, a group N(R¹⁰R¹¹) as defined above or a group SR¹² wherein R¹² is H or C₁-C₆ alkyl; or R⁷ and R⁸, when situated on adjacent carbon atoms, form together with the carbon atoms to which they are attached a benzene ring or a methylenedioxy substituent;
R⁹ is phenyl or an unsaturated heterocylic group, each of which is unsubstituted or substituted by C₁-C₆ alkyl, OH, C₁-C₆ alkoxy, halogen, C₁-C₆ cycloalkyl, phenyl, benzyl, trifluoromethyl, nitro, acetyl, benzoyl or N(R¹⁰R¹¹) as defined above, or two substituents on adjacent ring positions of the said phenyl or heterocyclic group together complete a saturated or unsaturated 6-membered ring or form a methylenedioxy group;

n is 0 or 1; and

m is 0 or an integer of 1 to 6;

or a pharmaceutically acceptable salt thereof.

Further derivatives as well as methods for producing said compound are detailed in WO 98/17648 which is, therefore, included herein in its entirety by reference.

However, a compound of the following formula is preferred:

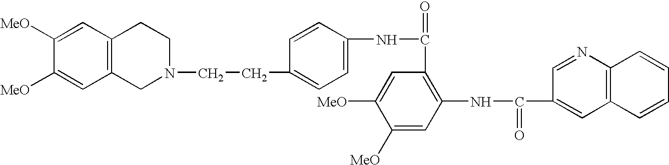

### S-9788; EP-B1 0 466 586

Polymethyleneimines of the general formula I;

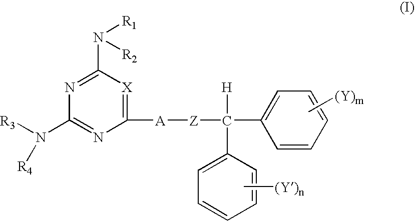

wherein

a) X represents the group CH or a nitrogen atom;

b) A represents a polymethyleneimines group of the formula

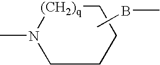

wherein:
B is a sulphur atom or a radical NR′ wherein R′ represent a hydrogen atom or a methyl radical,
and
q is 2;

c) each of R₁, R₂, R₃ and R₄, which are identical or different, represents:
a hydrogen atom, an allyl radical or a propyl radical, with the proviso that at least one of the groups

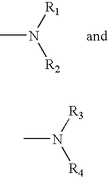

represents allylamino radical;

d) Z represents a or ethylene radical;

e) each of Y and Y′, which are identical or different, represents a hydrogen or fluorine atom or a methyl or methoxy radical;

f) each of m and n, which are identical or different, represents the integer 1 or 2;

and the corresponding diastereoisomers and enantiomers.

Further derivatives as well as methods for producing said compound are detailed in EP-B1 0 466 586 which is, therefore, included herein in its entirety by reference.

OC-144-093; U.S. Pat. No. 5,756,527

Imidazol derivatives having formula 1

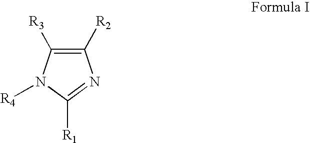

wherein:
R₁ is selected from the group consisting of: mono- di- and tri-substituted phenyl or thienyl, the substituents are selected from the group consisting of:
   (i) substituted C₁₋₆ alkyl, substituted C₂₋₆ alkyloxy, wherein the substituents are selected from the group consisting of hydrogen or C₁₋₆ alkoxy;
   (ii) C₁₋₁₁ CO₂R₅, trans-CH═CHCO₂R₅, wherein R₅ is C₁₋₁₁ alkyl, or phenyl C₁₋₁₁ alkyl;
R₂ and R₃ are mono-, di, and tri-substituted phenyl wherein the substituents are independently selected from:
   (i) halo;
   (ii) C₁₋₆ allyl-amino, or di(C₁₋₆ alkyl)amino, and R₄ is hydrogen.

Further derivatives as well as methods for producing said compound are detailed in U.S. Pat. No. 5,756,527 which is, therefore, included herein in its entirety by reference.

### Elacridar (GF-120918); EP-A1 0 494 623

A compound of formula (I)

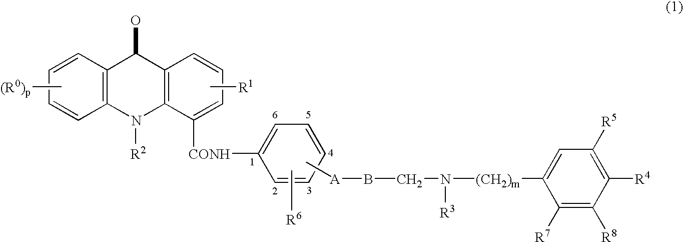

wherein R^{o} represents a hydrogen or halogen atom or a C₁₋₄alkyl, C₁₋₄alkoxy, C₁₋₄alkylthio, amino or nitro group;
p represents 1; or when R^{o} represents C₁₋₄alkoxy may also represent 2 or 3;
R¹ represents a hydrogen or halogen atom, or a C₁₋₄alkyl, C₁₋₄alkoxy or C₁₋₄alkylthio group;
R² represents a hydrogen atom or a C₁₋₄alkyl group
A represents an oxygen or a sulphur atom, a bond or a group (CH₂)₁ NR⁹ (where 1 represents zero or 1 and R⁸ represents a hydrogen atom or a methyl group);
B represent a C₁₋₄ alkylene chain optionally substituted by a hydroxyl group except that the hydroxyl group and moiety A cannot be attached in the same carbon atom when A represents an oxygen or sulphur atom or a group (CH₂)ₘNR⁸, or when A represents a bond B may also represent a C₂₋₄alkenylene chain;
R³ represents a hydrogen atom or a C₁₋₄alkyl group;
m represents 1 or 2;
R⁴ represents a hydrogen atom or a halogen atom, or a C₁₋₄alkyl, C₁₋₄alkoxy or C₁₋₄alkylthio group;
R⁵ represents a hydrogen atom or a C₁₋₄alkoxy group;
R⁶ represents a hydrogen atom or a C₁₋₄alkyl or C₁₋₄alkoxy group;
R⁷ represents a hydrogen atom or R³ and R⁷ together form a group —(CH₂)ₘ— where n represents 1 or 2;
R³ represents a hydrogen atom or a C₁₋₄alkoxy group; the group

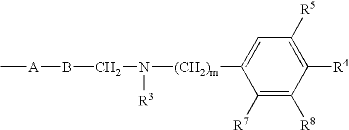

is attached at the benzene ring 3 or 4 position relate to the carboxamide substituent provided that when the group is attached at the benzene ring 3 position then R⁵ must be attached at the benzene ring 6 position;
and salts and solvates thereof.

Further derivatives as well as methods for producing said compound are detailed in EP-A1 0 494 623 which is, therefore, included herein in its entirety by reference.

MS 209; U.S. Pat. No. 5,405,843; EP 0 363 212 B1

A compound of the general formula [I]

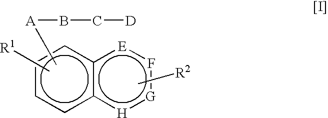

in which A represents an oxygen or sulfur atom or a methylene amino or —NR³ group, which is bound to any available position on the condensed benzene ring; B represents —(CH₂)ₙ—,

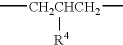

or —CO(CH₂)ₙ—; C represents

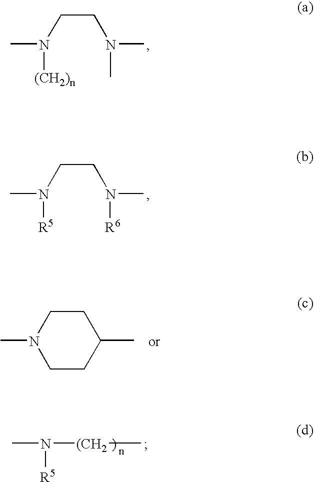

D represents

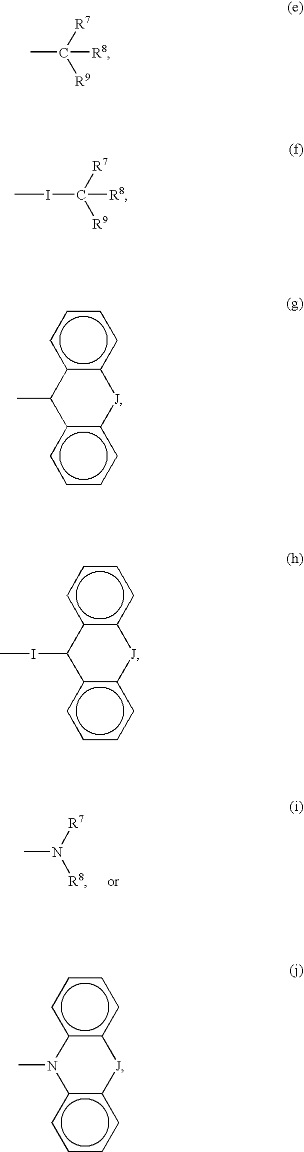

C and D can together form

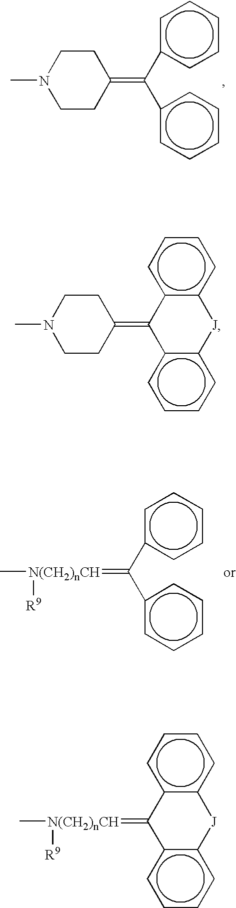

E, F, G, and H each independently represent a carbon or nitrogen atom, provided that either one or two of them is nitrogen, R¹ and R² each independently represent a hydrogen or halogen atom, a C₁₋₄ alkyl, amino group, substituted amino group, a C₁₋₄ alkoxy, C₁₋₄ alkylthio, C₁₋₄ alkylsulfonyl, trifluoromethyl, cyano, nitro, amino or hydroxy group, wherein R¹ and R² may be on any position available on the condensed ring or one each on each of the rings or both on the same ring of which the condensed ring is formed, R³ represents a hydrogen atom or a C₁₋₄ alkyl or acyl group; R⁴ represents a hydroxyl lower alkylamine (where alkyl) is C₁₋₄), C₁₋₄ alkoxyl or C₁₋₂ acyloxy group, R⁵ and R⁶ each independently represent a hydrogen atom or a C₁₋₄ alkyl or hydroxyalkyl group; R⁷, R⁸ and R⁹ each independently represent a hydrogen atom or a hydroxy, phenyl, pyridyl or substituted phenyl group, I represents an oxygen atom,

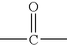

or a nitrogen atom;
J represents —(OH₂)₂—, —CH═CH—, —OCH₂— or an oxygen atom; n represents an integral number in the range between 1 and 10, and m represents an integral number 0, 1 or 2, or a pharmaceutically acceptable salt thereof;
   with the proviso that if C represents (a) or (b) than D does not represent (i) or (j) and I does not represent a nitrogen atom.

Further derivatives as well as methods for producing said compound are detailed in U.S. Pat. No. 5,405,843 or EP 0 363 212 B1 which is, therefore, included herein in its entirety by reference.

### BIBW-22; (DE 4225353)

A compound of the formula:

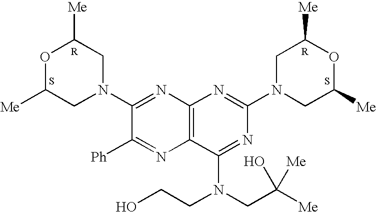

Further derivatives as well explanations of the indicated residues and methods for producing said compound are detailed in DE 4225353 which is, therefore, included herein in its entirety by reference.

### Lysodren®

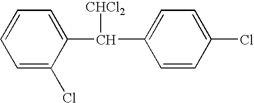

### 1,1-dichloro-2-(o-chlorophenyl)-2-(p-chlorophenyl)ethane

### Zaprinast®

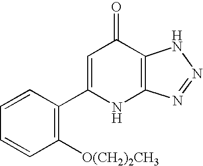

### 1,4-Dihydro-5-(2-propoxyphenyl)-7H-1,2,3-triazolo[4,5-d]pyrimidine-7-one

Other inhibitors which are within the scope of the present invention are detailed e.g. in WO 98/48784 which is therefore incorporated herein by reference. It has to be understood that some of the inhibitor(s) mentioned before are identical with the following list of inhibitors which is exemplary and provides sufficient information concerning the chemical nature of the inhibitor(s) of the invention. In particular, WO 98/48784 discloses desmethoxyVerapamil, quinine, chinchonidine, primaquine, tamoxifen, dihydrocyclosporin, yohimbine, corynanthine, reserpine, physostigmine, acridine, acridine orange, quinacrine, trifluoroperazine chlorpromazine, propanolol, atropine, tryptamine, forskolin, 1,9-dideoxyforskolin, cyclosporin, (USPatent 4,117,118 (1978)), PSC-833 (cyclosporin D, 6-[(2S, 4R, 6E)-4-methyl-2 (methylamino)-3-oxo-6-octenoic acid]-(9CI)), [U.S. Pat. No. 5,525,590] [ACS121584-187], Keller et al., “SDZ PSC 833, a non-immunosuppressive cylcosporine: its potency in overcoming p-glycoprotein-mediated multidrug resistance of murine leukemia”, Int J Cancer 50:593-597 (1992)), RU-486 (17-hydroxy-11 p-[4-dimethylaminophenyl]-17a prop-1-ynyl estra-4,9-dien-3 one), RU-49953 (17P-hydroxy-11P, 17a-[4-dimethylaminophenyl]-17a prop-1-ynyl estra-4, 9 dien-3 one), S9778 (6-{4-[2,2-di( ) ethylamino]-1-piperidinyl}-N,N′, di-2-propenyl-1,3,5-triazine-2,4-diamine, bismethane sulfonate, [U.S. Pat. No. 5,225,411; EP 466586] [ACS # 140945-01-3]; Dhainaut et al., “New triazine derivatives as potent modulators of multidrug resistance,” J Medicinal Chemistry 35:2481-2496 (1992)), MS-209 (5-[3-[4-(2,2-diphenylacetyl)piperazin-1-yl]2-hydroxypropoxy]quinoline sesquifumarate, [U.S. Pat. No. 5,405,843 (continuation of U.S. Pat. No. 5,112,817)], [ACS # It 158681-49-3], Sato et al., “Reversal of multidrug resistance by a novel quinoline derivative, MS-209, Cancer Chemother Pharmacol 35:271-277 (1995)), MS-073 (Fukazawa et al., European Patent Application 0363212 (1989)), FK-506 (Tanaka et al., M. Physicochemical properties of FK-506, a novel immunosuppressant isolated from Streptomyces tsukubaensis” Transplantation Proceedings. 19(5 Suppl 6):11-6, (1987); Naito et al., “Reversal of multidrug resistance by an immunosuppressive agent FK-506,” Cancer Chemother & Pharmacol. 29:195-200 (1992); Pourtier-Manzanedo et al., “FK-506 (fujimycin) reverses the multidrug resistance of tumor cells in vitro,” Anti-Cancer Drugs 2:279-83 (1991); Epand & Epand, “The new potent immunosuppressant FK-506 reverses multidrug resistance in Chinese hamster ovary cells,” Anti-Cancer Drug Design 6:189-93 (1991)), VX-710 (2-peperidinecarboxylic acid, 1-[oxo(3,4,5-trimethoxyphenyl)acetyl]-3-(3-pyridinyl)-1-[3-(3-pyridinyl)propyl]butyl ester [ACS159997-94-1] [U.S. Pat. No. 5,620,971]Germann et al., “Chemosensitization and drug accumulation effects of VX-710, Verapamil, cyclosporin A, MS-209 and GF120918 in multidrug resistance-associated protein MRP” Anti-Cancer Drugs 8, 141-155 (1997); Germann et al., “Cellular and biochemical characterization of VX-710 as a chemosensitizer: reversal of Pglycoprotein-mediated multidrug resistance in vitro” Anti-Cancer Drugs 8, 125-140 (1997)), VX-853 ([U.S. Pat. No. 5,543,423] [ACS It 190454-58-1), AHC-52 (methyl 2-(N-benzyl-N-methylamino)ethyl-2,6-dimethyl-4-(2-isopropylpyrazolo[1,5-a]pyridine-3-yl)-1,4-dihyropyridine-3,5-dicarboxylate; [Japanese Patent 63-135381; European Patent 0270926] [ACS 119666-09-0] Shinoda et al., “In vivo circumvention of vincristine resistance in mice with P388 leukemia using a novel compound, AHC-52,” Cancer Res 49:1722-6 (1989)), GF-120918 (9,10-dihydro-5-methoxy-9-oxo-N-[4-[2-(1,2,3,4-tetrahydro-6,7-dimethoxyisoquinol-2-yl)ethyl]phenyl]-4 acridinecarboxamide,[U.S. Pat. No. 5,604,237] [ACS It 143664-11-3] Hyafil et al., “In vitro and in vivo reversal of multidrug resistance by GF 120918, an acridonecarboxamide derivative,” Cancer Res 53:4595-4602 (1993)), and XR-9051 (3-[(3Z,6Z)-6-Benzylidene-1-methyl-2,5-dioxopiperazin-3-ylidenemethyl]-N-[4-[2-(6,7-dimethoxy-1,2,3,4-tetrahydroisoquinolin-2-yl)ethyl]phenyl]benzamide hydrochloride, [AC Slt57-22-7]). Other inhibitors are for example listed in WO 99/17757, which is therefore incorporated herein by reference. As mentioned above, reference may be made to the literature for sources of appropriate ABC-transporter inhibitors to use according to the invention.

Thus, it has to be understood that also other ABC-transporter inhibitors which fulfill the above criteria (i.e. which are capable of reducing the transport of hyaluronan across a lipid bilayer as mediated by ABC-transporter(s)) are within the scope of the present invention, as are their derivatives e.g. functional derivatives and analogues, and any isomers (e.g. stereoisomers and/or enantiomers) thereof. Also included are the salts of the inhibitor(s) as mentioned herein, including both organic and inorganic salts (e.g. with alkali and alkaline earth metals, ammonium, ethanolamine, diethanolamine and meglumine, chloride, hydrogen carbonate, phosphate, sulphate and acetate counterions). Appropriate pharmaceutically acceptable salts, are well described in the pharmaceutical literature. In addition, some of these salts may form solvates with water or organic solvents such as ethanol. Such solvates are also included within the scope of this invention.

In another aspect of the methods and uses of the present invention, the inhibitor is an antibody, preferably an antibody the binding of which interferes with the transport of hyaluronan mediated by the ABC-transporters of this invention. It is envisaged that the antibody specifically recognizes the ABC-transporter to whom it is directed to, i.e. the antibody shows no or essentially no cross-reactivity to other proteins and/or other ABC-transporter(s). For example a monoclonal antibody against P-glycoprotein (C219 from Calbiochem) [70] was used to verify the participation of the MDR transporter in hyaluronan export. Membranes from this cell line were incubated with and without the antibody and then assayed for hyaluronan transport activity. The antibody decreased the hyaluronan transport activity by 20%. Other antibodies which are within the gist of the present invention are well known to the skilled person and are exemplified by the following non-limiting selection: Anti-P-Glycoprotein (4E3), Human (Mouse); Anti-P-Glycoprotein (C219), Hamster and Human (Mouse); Anti-P-Glycoprotein (C494), Hamster and Human (Mouse); Anti-P-Glycoprotein (JSB-1), Hamster (Mouse); Anti-P-Glycoprotein (4E3), Human (Mouse); Anti-P-Glycoprotein (C219), Hamster and Human (Mouse); Anti-P-Glycoprotein (C494), Hamster and Human (Mouse); Anti-P-Glycoprotein (JSB-1), Hamster (Mouse); Anti-P-Glycoprotein, Human (Rabbit). All the above listed antibodies are manufactured by Calbiochem. Other antibodies which are specific for an ABC-transporter can be easily produced by methods well-known in the art. For example it is possible to use cell lines secreting antibody to essentially any desired substance that produces an immune response. RNA encoding the light and heavy chains of the immunoglobulin can then be obtained from the cytoplasm of the hybridoma. The 5′ end portion of the mRNA can be used to prepare cDNA to be inserted into an expression vector. The DNA encoding the antibody or its immunoglobulin chains can subsequently be expressed in cells, preferably mammalian cells. Depending on the host cell, renaturation techniques may be required to attain proper conformation of the antibody. If necessary, point substitutions seeking to optimize binding or stability of the antibody may be made in the DNA using conventional cassette mutagenesis or other protein engineering methodology such as is disclosed herein. Furthermore, antibodies or fragments thereof to the aforementioned ABC-transporter(s) can be obtained by using methods which are described, e.g., in Harlow and Lane “Antibodies, A Laboratory Manual”, CSH Press, Cold Spring Harbor, 1988. Other suitable antibodies as well as methods for testing the effectivness of such antibodies are detailed in WO02071 061.

For the production of antibodies in experimental animals, various hosts including goats, rabbits, rats, mice, and others, may be immunized by injection with polypeptides of the present invention or any fragment or oligopeptide or derivative thereof which has immunogenic properties. Techniques for producing and processing polyclonal antibodies are known in the art and are described in, among others, Mayer and Walker, eds., “Immunochemical Methods in Cell and Molecular Biology”, Academic Press, London (1987). Polyclonal antibodies also may be obtained from an animal, preferably a mammal, previously infected with the virus of the invention. Methods for purifying antibodies are known in the art and comprise, for example, immunoaffinity chromatography. Depending on the host species, various adjuvants or immunological carriers may be used to increase immunological responses. Such adjuvants include, but are not limited to, Freund's, complete or incomplete adjuvants, mineral gels such as aluminium hydroxide, and surface active substances such as lysolecithin, pluronic polyols, polyanions, peptides, oil emulsions and dinitrophenol. An example of a carrier, to which, for instance, a peptide of the invention may be coupled, is keyhole limpet hemocyanin (KLH). When derivatives of said antibodies are obtained by the phage display technique, surface plasmon resonance as employed in the BIAcore system can be used to increase the efficiency of phage antibodies which bind to an epitope of the peptide or polypeptide of the invention (Schier, Human Antibodies Hybridomas 7 (1996), 97-105; Malmborg, J. Immunol. Methods 183 (1995), 7-13). In many cases, the binding phenomena of antibodies to antigens is equivalent to other ligand/anti-ligand binding.

The production of chimeric antibodies is described, for example, in WO89/09622. A further source of antibodies to be utilized in accordance with the present invention are so-called xenogenic antibodies. The general principle for the production of xenogenic antibodies such as human antibodies in mice is described in, e.g., WO 91/10741, WO 94/02602, WO 96/34096 and WO 96/33735.

The production of recombinant antibodies is described, for example, in R. Kontermnn, S. Dujbel: Antibody Engineering, Springer Lab Manual 2001.

In a preferred embodiment, the antibody of the invention has an affinity of at least about 10⁻⁷ M, preferably at least about 10⁻⁸ M more preferably at least about 10⁻⁹ M and most preferably at least about 10⁻¹⁰ M.

The antibody which is used in accordance with the uses or methods of the invention may be a monoclonal or a polyclonal antibody (see Harlow and Lane, “Antibodies, A Laboratory Manual”, CSH Press, Cold Spring Harbor, USA, 1988) or a derivative of said antibody which retains or essentially retains its binding specificity. Preferred derivatives of such antibodies are chimeric antibodies comprising, for example, a mouse or rat variable region and a human constant region.

The term “specifically recognizing” in connection with the antibody etc (functional fragments and so on) used in accordance with the present invention means that the antibody etc. does not or essentially does not cross-react with polypeptides of similar structures. Cross-reactivity of a panel of antibodies etc. under investigation may be tested, for example, by assessing binding of said panel of antibodies etc. under conventional conditions to the polypeptide of interest as well as to a number of more or less (structurally and/or functionally) closely related polypeptides. Only those antibodies that bind to the polypeptide of interest (i.e to an ABC-transporter which transports hyaluronan across a lipid bilayer) but do not or do not essentially bind to any of the other polypeptides which are preferably expressed by the same tissue as the polypeptide of interest (e.g. in a chondrocyte or in cells which are comprised in cartilage and which are responsible for the hyaluronan synthesis) are considered specific and are, therefore, selected for further studies in accordance with the invention.

The term “functional fragment” as used herein refers to fragments of the antibodies as specified herein which retain or essentially retain the binding specificity of the antibodies like, separated light and heavy chains, Fab, Fab/c, Fv, Fab′, F(ab′)2. The term “antibody” also comprises bifunctional antibodies and antibody constructs, like single chain Fvs (scFv) or antibody-fusion proteins. The term “scFv fragment” (single-chain Fv fragment) is well understood in the art and preferred due to its small size and the possibility to recombinantly produce such fragments. It is also envisaged in context of this invention that the term “antibody” comprises antibody constructs which may be expressed in cells, e.g. antibody constructs which may be transfected and/or transduced via, inter alia, viruses or vectors. It is in particular envisaged that such antibody constructs specifically recognize the polypeptides of the present invention. It is, furthermore, envisaged that said antibody construct is employed in gene therapy approaches.

In a particularly preferred embodiment of the method of the invention, said antibody or antibody binding portion is or is derived from a human antibody or a humanized antibody. The term “humanized antibody” means, in accordance with the present invention, an antibody of non-human origin, where at least one complementarity determining region (CDR) in the variable regions such as the CDR3 and preferably all 6 CDRs have been replaced by CDRs of an antibody of human origin having a desired specificity. Optionally, the non-human constant region(s) of the antibody has/have been replaced by (a) constant region(s) of a human antibody. Methods for the production of humanized antibodies are described in, e.g., EP-A1 0 239 400 and WO90/07861. The specifically binding antibody etc. may be detected by using, for example, a labeled secondary antibody specifically recognizing the constant region of the first antibody. However, in a further particularly preferred embodiment of the method of the invention, the antibody or derivative of said antibody itself is detectably labeled at the binding portion. Detectable labels include a variety of established labels such as radioactive (¹²⁵I, for example) or fluorescent labels (see, e.g. Harlow and Lane, loc. cit.). Binding may be detected after removing unspecific labels by appropriate washing conditions (see, e.g. Harlow and Lane, loc. cit.).

In addition to ABC-transporter antibodies and functional antibody fragments, small molecule peptidomimetics or non-peptide mimetics can be designed to mimic the action of the ABC-transporter antibodies in inhibiting or modulating the transport of hyaluronan, presumably by interfering with the action of said ABC-transporter(s). Methods for designing such small molecule mimics are well known (see, for example, Ripka and Rich, Curr. Opin. Chem. Biol. 2: 441-452,1998; Huang, et al., Biopolymers 43: 367-382,1997; al-Obeidi, et al., Mol. Biotechnol. 9: 205-223,1998). Small molecule inhibitors that are designed based on the ABC-transporter antibody may be screened for the ability to interfere with the ABC-transporter—ABC-transporter-antibody binding interaction. Candidate small molecules exhibiting activity in such an assay may be optimized by methods that are well known in the art, including for example, in vitro screening assays, and further refined in in vivo assays for inhibition or modulation of ABC-transporter-mediated hyaluronan-transport by any of the methods described herein or as are well known in the art. Such small molecule inhibitors of the ABC-transporter action (transport of hyaluronan) should be useful in the present uses and/or methods for treating and/or preventing a disease which is associated with an excess transport of hyaluronan across a lipid bilayer, e.g. arthritis and/or for screening compounds which might be useful as new lead compounds for the generation of more effective ABC-transporter inhibitors.

In another preferred embodiment of the uses and methods of the present invention, the inhibitor is an anti-sense, iRNA, siRNA or ribozyme. An siRNA approach is, for example, dislosed in Elbashir ((2001), Nature 411, 494-498)). It is also envisaged in accordance with this invention that for example short hairpin RNAs (shRNAs) are employed in accordance with this invention as inhibitors. The shRNA approach for gene silencing is well known in the art and may comprise the use of st (small temporal) RNAs; see, inter alia, Paddison (2002) Genes Dev. 16, 948-958. Approaches for gene silencing are known in the art and comprise “RNA”-approaches like RNAi or siRNA. Successful use of such approaches has been shown in Paddison (2002) loc. cit., Elbashir (2002) Methods 26, 199-213; Novina (2002) Mat. Med. Jun. 3, 2002; Donze (2002) Nucl. Acids Res. 30, e46; Paul (2002) Nat. Biotech 20, 505-508; Lee (2002) Nat. Biotech. 20, 500-505; Miyagashi (2002) Nat. Biotech. 20, 497-500; Yu (2002) PNAS 99, 6047-6052 or Brummelkamp (2002), Science 296, 550-553. These approaches may be vector-based, e.g. the pSUPER vector, or RNA polIII vectors may be employed as illustrated, inter alia, in Yu (2002) loc. cit.; Miyagishi (2002) loc. cit. or Brummelkamp (2002) loc. cit. Furthermore, the appended examples proof the feasability of the specific RNAi-mediated dorwnregulation of an ABC-transporter (see Example 10). “Anti-sense” and “antisense nucleotides” means DNA or RNA constructs which block the expression of the naturally occurring gene product. As used herein, the terms “antisense oligonucleotide” and “antisense oligomer” are used interchangeably and refer to a sequence of nucleotide bases that allows the antisense oligomer to hybridize to a target sequence in an RNA by Watson Crick base pairing, to form an RNA: oligomer heteroduplex within the target sequence. The term “target sequence” in this regard refers to the sequence of the ABC-transporters as described herein; the Accession numbers of the human ABC-transporters are e.g. depicted in FIG. 1; other (e.g. non-human) ABC-transporters are well known to the skilled person and can be easily searched for example on http://www.ncbi.nim.nih.gov/entrez/query.fcqi?db=PubMed. The oligomer may have exact sequence complementarity to the target sequence or near complementarity. Such antisense oligomers may block or inhibit translation of the mRNA containing the target sequence, or inhibit gene transcription, may bind to double-stranded or single stranded sequences. Preferably, said antisense oligonucleotides as used herein are “nuclease-resistant” oligomeric molecule e.g. their backbone is not susceptible to nuclease cleavage of a phosphodiester bond. Exemplary nuclease resistant antisense oligomers are oligonucleotide analogs, such as phosphorothioate and phosphate-amine DNA (pnDNA), both of which have a charged backbone, and methylphosphonate, morpholino, and peptide nucleic acid (PNA) oligonucleotides, all of which may have uncharged backbones.

In accordance with the present invention, the term “aptamer” means nucleic acid molecules that can bind to target molecules. Aptamers commonly comprise RNA, single stranded DNA, modified RNA or modified DNA molecules. The preparation of aptamers is well known in the art and may involve, inter alia, the use of combinatorial RNA libraries to identify binding sides (Gold, Ann. Rev. Biochem. 64 (1995), 763-797).

Arthritis is, as mentioned before, accompanied with a loss of cartilage at the joint surface. The cartilage goes through different stages during pathogenesis. At first chondrocytes try to replace loss of cartilage by increased synthesis and proliferation; simultaneously lacunae of edema and increased water binding occurs which leads to softening of the cartilage matrix. At the second stage new cartilage production cannot compensate for the loss and at the third stage loss of cartilage is complete.

Thus, in a further embodiment of the uses and methods of the present invention said arthritis is characterized by a degeneration and/or a destruction of cartilage. The term “degeneration and/or destruction of cartilage” includes within the meaning of the present invention dysregulation of turnover and repair of joint tissue. The pathological features are focal areas of destruction of articular cartilage associated with hypertrophy of the subcondral bone, joint margin and capsule. The radiological changes include joint space narrowing, subchondral sclerosis and cysts, pain, loss of joint motion and disability.

In a further embodiment of the methods and uses of the present invention said arthritis is osteoarthritis, (juvenile) chronic arthritis, rheumatoid arthritis, psoriatic arthritis, A. mutilans, septic arthritis, infectious arthritis and/or reactive arthritis. Thus, the term “arthritis” as used herein includes all forms of arthritis. First, the primary or idiopathic form. The secondary forms such as metabolic disorders such as ochronosis, acronmegaly, hemochromatosis and calcium crystal deposition and gout or apatite deposition; anatomic derangements such as slipped epiphysis, epiphysial dysplasias, Blount's disease, Legge-Perthe disease, congetial dislocation of the hip, leg length inequality, hypermobility syndromes; traumatic causes such as major joint trauma, fracture through a joint or osteonecrosis, joint surgery, chronic injury; any inflammatory arthropathy such as (juvenile) chronic arthritis, rheumatoid arthritis, psoriatic arthritis, A. mutilans, septic arthritis, infectious arthritis and/or reactive arthritis; joint abnormalities in thyroid diseases, diabetes melitus, hemophilia, amyloidosis, dialysis arthropathies, primary hyperlipidemias and xanthomatosis, Gaucher's disease, mucopolysaccharidosis; metabolic, regional and heritable bone and joint diseases such as osteoporosis, osteomalacia, renal bone diseases, algodystrophyireflex sympathetic dystrophy syndrom, Paget's disease, hypertrophic osteoarthopathy, tumors of bone, heritable collagen disorders, hypermobility syndrome, joint dysplasias [96]. The respective diseases as well as their symptoms are well-known to the skilled person and e.g. derivable from textbooks like Pschyrembel et al. or the like. Osteoarthritis (also known as osteoarthrosis or “non-inflammatory arthritis”) is a type of arthritis that is caused by the breakdown and eventual loss of the cartilage of one or more joints. Rheumatoid arthritis is an autoimmune disease that causes chronic inflammation of the joints. Rheumatoid arthritis can also cause inflammation of the tissue around the joints, as well as other organs in the body.

The present invention relates to the use of an inhibitor of at least one ABC-transporter capable of transporting hyaluronan across a lipid-bilayer, for the preparation of a pharmaceutical composition for the treatment of osteoarthritis. In a preferred embodiment said at least one ABC-transporter is MRP5 (ABCC5), ABCC11 and/or ABCC12. In a more preferred embodiment said at least one ABC-transporter is MRP5 (ABCC5).

In another preferred embodiment the present invention relates to the use of Zaprinast® for the preparation of a pharmaceutical composition for the treatment of arthritis, preferably rheumatoid arthritis or osteoarthritis.

In another preferred embodiment the present invention relates to the use of Elacridar (GF-120918), Valspodar (PSC-833), Bericodar (VX-710), Tariquidar (XR-9576), S-9788, Ly-335979, OC-144-093 and/or Lysodren® for the preparation of a pharmaceutical composition for the treatment of osteoarthritis.

The inhibitors of the present invention can be applied prophylactically with subjects that have or might have an enhanced individual risk factor such as obesity, heredity, for women after the menopause, osteoporosis, hypermobility, for persons with distorted joint shape or for persons with repetitive use of particular joint groups. Prophylactic treatment will be especially important for those diseases that will lead to an inflammation. Thus immediately after a heart attack it is expected the tissue necrosis may be a consequence. In this situation immediate prevention of increased hyaluronan production will reduce further complications. Similarly, organ transplantation will certainly increase hyaluronan production. Also in this case, reduction of hyaluronan transport will be beneficial.

Thus in a further embodiment of the uses of the present invention said inhibitor(s) is(are) to be administered prophylactically.

Alternatively, the inhibitors can by applied therapeutically as early as possible e.g. with respect to arthritis after diagnosis of joint insult to inhibit further destruction, to support self regeneration and restore joint function.

Thus, in another embodiment of the uses of the present invention said inhibitor(s) is(are) to be administered therapeutically.

The dosage regimen utilising the inhibitors or screened compounds(inhibitors) of the present invention is selected in accordance with a variety of factors including type, species, age, weight, sex and medical condition of the patient; the severity of the condition to be treated; the route of administration; and the particular compound employed. It will be acknowledged that an ordinarily skilled physician or veterinarian can easily determine and prescribe the effective amount of the compound required to prevent, counter or arrest the progress of the condition.

It is also envisaged that the inhibitors of the present invention are employed in co-therapy approaches, i.e. in co-administration with other medicaments or drugs, for example other drugs for preventing, treating or ameliorating arthritis which are known in the art (e.g. hyaluronan injections like Hyalgan® (Sanofi Pharmaceuticals), Orthovisc® (Anika Therapeutics) and SynVisc® (Biomatrix, now Genzyme), anti-inflammatory drugs and so on). It will be appreciated that these list of co-administered drugs is not limiting but severs as an example only. The skilled person is of course well-aware of suitable drugs which have a beneficial effect on arthritis and, therefore, might be useful when co-administered with the inhibitor(s) as described herein. With respect to the other diseases mentioned herein before (ischemic or inflammatory edema; tumors which are characterized by an overproduction of hyaluronan such as melanoma, mesothelioma or colon carcinoma; lump formation after contusion or insect bites; injuries/conditions which are followed by inflammation and hyaluronan overproduction like heart infarct, alveolitis, pancreatitis, pulmonary or hepatic fibrosis, radiation induced inflammation, Crohn's disease, myocarditis, scleroderma, psoriasis, sarcoidosis), it is envisaged that the inhibitor(s) of the present invention are co-administered e.g. together with cytostatics for the treatment of tumors and/or with antiinflammatory drugs for treatment of inflammations or inflammatory or ischemic edema and so on. Suitable compounds in this regard are well-known to the skilled artisan.

The combination of other drugs with the ABC transport inhibitors will be particularly important for the secondary forms of arthritis described above, since in theses cases they can optimally exert their beneficial properties, if the primary cause of cartilage destruction is also eliminated. For septic arthritis it should be combined with antibiotics, for the inflammatory forms of arthritis with corticosteroids and immunosuppressives.

Based on the finding that the inhibition of an ABC-transporter specifically reduces or abolishes the transport of hyaluronan across a lipid bilayer as mentioned before, the methods of the invention allow the convenient identification and/or isolation of compounds that counteract the transport of hyaluronan mediated by ABC-transporters such that a normal transport of hyaluronan is restored or essentially restored. The meaning of a “normal” transport of hyaluronan was explained herein above. Thus, the present invention opens up the possibility to screen for compounds which reduce the transport of hyaluronan as mediated by ABC-transporter(s) in a cell/tissue/subject and, thereby, are suitable for treating or preventing a disease which is associated with an excess transport of hyaluronan across a lipid bilayer, e.g. arthritis in a subject. These methods can be applied to screen for efficient drugs for the treatment/prevention of a disease which is associated with an excess transport of hyaluronan across a lipid bilayer, e.g. arthritis.

The term a compound “which is suitable for the treatment of a disease which is associated with an excess transport of hyaluronan across a lipid bilayer, e.g. arthritis” as used herein defines a compound which prevents or ameliorates a disease which is associated with an excess transport of hyaluronan across a lipid bilayer, e.g. arthritis, i.e. which has a beneficial effect (e.g. reduction of the overall transport of hyaluronan across a lipid-bilayer; reduction of the destruction of cartilage; maintenance of the actual state of destruction of cartilage; prevention of a further destruction of the cartilage etc; prevention of fluid accumulation in edema, prevention of tissue softening that is required for invasion of inflammatory cells or for metastasis, reduction of cell growth of tumors. The term “arthritis” has been defined elsewhere in this specification.

Suitable test-assays for measuring the specific inhibition of the hyaluronan transport as mediated by ABC-transporter(s) (as specified herein) can be carried out as follows. It has to be understood that these methods are exemplary only and are not intended to limit the scope of the present invention.

The specific transport can be measured in single cells by introducing labelled hyaluronan into the cell, e.g. by microinjection or otherwise as described herein and, e.g in [115]. A suitable label is e.g. a fluorescent tag. However, also other tags (described elsewhere herein) may be used. It is also envisaged to use derivatives and the like of hyaluronan. Said derivatives which can be used instead of hyaluronan have been described elsewhere in this specification (e.g. identified as “indicator compounds” or “substrate analogs”). After injection into the cell, e.g the disappearance of fluorescence under the influence of hyaluronan transport inhibitors from the cytosol can directly be observed by a fluorescence microscope. The quantification of other labels will depend on the respective label used. The corresponding methods for detecting such labels are well-known in the art. As mentioned elsewhere before, it is also envisaged to employ cells which per se do not express/contain hyaluronan synthase, but which contain one or more ABC-transporter(s). It is, however, also envisaged to use cells which contain a hyaluronan-synthase—in such particular cases, the hyaluronan synthase can be inhibited e.g. by suitable antisense constructs, iRNA, siRNA, antibodies and/or other inhibitors which are well known to the skilled artisan.

Another specific screening method for inhibitors of hyaluronan transport is based on the introduction of partially degraded [¹⁴C]hyaluronan into a cell line, preferably a human cell line by lipofectamine. It will be appreciated that of course other lipid formulations such as lipofectin, the SuperFect Transfection Reagent from Qiagen, DOPSA or DOPE are similarly applicable.

[¹⁴C]hyaluronan is obtained by enzymatic synthesis from streptococcal membranes. The group C Streptococcus D181 strain is grown over night in TH-medium, diluted with fresh medium at a ratio of 1:3 and grown for another 3 hours. A suspension of the bacteria (10 ml) are subjected to ultrasonification (1 min 40 Watts) to disrupt the cells and sedimented by centrifugation at 10.000 g for 5 min. The sediment is washed with 50 mM TRIS-malonate pH 7.0 and incubated with 0.3 ml of substrate for hyaluronan synthesis (160 μM UDP-GlcNac and 8 μM UDP-[¹⁴C]GlcA (specific activity 320 mCi/mmol, 1 mM dithiothreitol, 10 mM MgCl₂, 0.15 M NaCl) and incubated for 4 h at 37° C. [¹⁴C]hyaluronan labelled hyaluronan is separated by gel filtration on a short column of Sephadex G-25 with the serum reduced medium Opti-MEM I from GIBCO as eluant and has a total radioactivity of 360.000 cpm. This solution was subjected to ultrasonification (1 min 40 Watts) to fragment the labelled hyaluronan.

Human cells are plated into microtiter plates and grown to 80-100% confluence. The medium was then changed to the solution of [¹⁴C]hyaluronan in Opti-MEM I as prepared above containing 5-10 μg/ml of lipofectamine and incubated for various times (1-72 hours) (100 μl/well). The cells are rinsed thoroughly and incubated in DMEM, 10% foetal calf serum. After different time periods 50 μl of the medium is withdrawn and its radioactivity is determined.

The screening methods of the present invention can, in general, be categorized into different groups, which, however does not mean that the methods of the present invention are also limited to the following particular groups. These and other methods are explained and exemplified herein as well as in the appended examples.
1. Determination of hyaluronan transport activity in isolated membranes of cells.
2. Assay of the hyaluronan concentration in media of cell cultures or organ cultures e.g. by radioactive incorporation of [3H]glucosamine, by ELISA or chemical assays of extracted hyaluronan.
3. Histological or immunohistological staining of hyaluronan in organ cultures or in tissues obtained from animal trails.

In order to demonstrate the specificity of the hyaluronan transport as mediated by ABC-transporter(s), i.e. to demonstrate that the mentioned ABC-transporter(s) is(are) mainly responsible for the absence/reduction of hyaluronan in the exterior of a cell (e.g. comprised in cell culture or in organ culture), it is envisaged that the respective cell culture or organ culture is pretreated with an inhibitor(s) of the hyaluronan synthase. Accordingly, any further reduction of the hyaluronan (or the respective analogues/derivatives of hyaluronan as described elsewhere) is specifically due to the reduction of the hyaluronan transport activity as mediated by one or more ABC-transporter(s). Alternatively, it is possible to determine the hyaluronan transport of a cell which is treated with an inhibitor of the hyaluronan synthase with the hyaluronan transport of the same type of cell (e.g. a chondrocyte) which is treated with such inhibitor(s) of hyaluronan synthase together with an inhibitor of the present invention. The comparison of the hyaluronan transport rate of both cells will allow the determination of the effect of the respective inhibitor of the ABC-transporter which was tested. Inhibitor(s) of hyaluronan synthase are well-known to the skilled artisan, e.g form [24-29]. Examples of such inhibitor(s) are exemplified in the following: Periodate-oxidized UDP-Glucuronic acid, periodate-oxidized UDP-N-acetyl-glucosamine [24,25]. Alternatively, it is also possible to reduce the content of hyaluronan synthase within the test-cell, e.g. by antisense, iRNA, siRNA and so on as described in [10]. The respective sequence of the hyaluronan synthase are well known to the skilled artisan. There are three human hyaluronan synthase with the Accession numbers NP_{—}005319 for hyaluronan synthase 2, NP_{—}619515 for hyaluronan synthase 3 isoform b, NP_{—}005320 for hyaluronan synthase 3 isoform a, NP_{—}001514 for hyaluronan synthase 1.

A further method which allows the measurement/quantification of the specific inhibition of the ABC-transporter is based on the fact that the ABC-transporter(s) are located in the cell-membrane, i.e. compounds which are not capable of entering a cell without further ado will only exert an inhibitory effect(s) on the ABC-transporter and e.g. not on the hyaluronan-synthase which is inside the cell. Accordingly, it is possible to test for the inhibitory action of the inhibitor(s) of the present invention simply by contacting the respective test-cell with a test-compound (inhibitor) and measuring the effect on the hyaluronan transport to the exterior of the cell. As mentioned elsewhere, it is also necessary to contact the test-cell with a suitable indicator-compound, i.e. to introduce the test-compounds by suitable methods like electro-chemical-poration; lipofection; bioballistics or microinjection. Subsequently, it is possible to evaluate the effect of the test-compound (inhibitor) on the hyaluronan-transport rate of an ABC-transporter in question, e.g. by measuring the quantity of indicator-compound in the exterior of the cell before and after the addition of the inhibitor to be tested.

In one embodiment, the present invention relates to a method for screening a compound which is suitable for the treatment of a disease which is associated with an excess transport of hyaluronan across a lipid bilayer, e.g. arthritis, said method comprising:
(a) contacting an isolated lipid bilayer comprising at least one ABC-transporter which is capable of transporting hyaluronan with a test compound and an indicator compound;
(b) measuring the effect of the test compound on the transport of the indicator compound across the lipid bilayer; and
(c) identifying test compounds which reduce the transport of the indicator compound.

The term “compound” which is interchangeable with “test compound” in accordance with the screening methods of the present invention shall mean any biologically active substance that has an effect on the transport of hyaluronan as mediated by ABC-transporter(s), whereas such compound has a positive or negative influence upon such ABC-transporter mediated hyaluronan-transport across a lipid bilayer. Preferred compounds are nucleic acids, preferably coding for a peptide, polypeptide, antisense RNA, iRNA, siRNA or a ribozyme or nucleic acids that act independently of their transcription respective their translation as for example an antisense RNA or ribozyme; natural or synthetic peptides, preferably with a relative molecular mass of about 1.000, especially of about 500, peptide analogs polypeptides or compositions of polypeptides, proteins, protein complexes, fusion proteins, preferably antibodies, especially murine, human or humanized antibodies, single chain antibodies, Fab fragments or any other antigen binding portion or derivative of an antibody, including modifications of such molecules as for example glycosylation, acetylation, phosphorylation, farnesylation, hydroxylation, methylation or esterification, hormones, organic or inorganic molecules or compositions, preferably small molecules with a relative molecular mass of about 1.000, especially of about 500.

The screening for inhibitors that are specific for a particular ABC transporter and that do not inhibit the synthase can be performed as already described above for HT29 and HT29-mdr. The gene for the ABC-transporter is cloned and transfected into a recipient cell line that produces hyaluronan. Both cell lines are then compared for their responsiveness toward the compound to be tested. Since the cell lines differ only in the overexpression of a certain ABC transporter, any difference in the response of the hyaluronan transport activity or in hyaluronan in increasing concentrations of the test compound can be attributed to a specific interaction between the inhibitor and the expressed transporter. Methods for over-expressing ABC-transporter(s) are well-known in the art and, additionally exemplified in this specification. Provided that the used test-cell comprises further ABC-transporter(s) it is envisaged that these ABC-transporter(s) is(are) identified (e.g. by antibody-mediated FACS-analysis or by RT-PCR as explained herein) and subsequently disrupted/blocked by e.g. antisense-constructs, iRNA, siRNA, antibodies and so on in order to specifically address the reduction of the hyaluronan transport to the over-expressed ABC-transporter. It is also envisaged that the hyaluronan synthase which might be active in these cells might be blocked/disrupted e.g. by any of the above mentioned methods (antisense, iRNA and so on).

An alternative method is the following procedure: If a ABC-transporter has been identified as a hyaluronan transporter (i.e. an ABC-transporter which is able to transport hyaluronan across a lipid bilayer) that in addition to hyaluronan also transports other substrates (a hypothesis that is very likely regarding the known broad substrate specificities of the transporters), it is feasible to find “substrate analogs” that will be transported. These “substrate analogs” are preferably labelled and can serve as indicator-compounds or probes for the inhibitory action of test-compounds to be tested. In fact, this scenario was already the starting point for the identification of inhibitors for hyaluronan production described here. Suitable labels and substrate analogs have been described elsewhere in this specification.

It is furthermore envisaged that the screening-methods described herein (e.g. the screening methods for screening a compound/inhibitor which is suitable for the treatment of a disease which is associated with an excess transport of hyaluronan across a lipid bilayer, e.g. arthritis) are carried out in the form of high-throughput methods. Such methods for screening for transdominant effector peptides and RNA molecules are for example described in WO 97/27213 or WO 97/27212 or EP-B1 0 832 207. Thus it has to be understood that the present invention also encompasses methods for screening for an inhibitor which is (i) capable of altering the hyaluronan-transporting phenotype of a cell (i.e. the inhibitor effects the hyaluronan-transport mediated by one or more ABC-transporter(s)) and thereby represents an inhibitor which is (ii) suitable for the treatment and/or prevention of a disease which is associated with an excess transport of hyaluronan across a lipid bilayer, e.g. arthritis. The methods comprise the steps of a) introducing a molecular library of randomized candidate nucleic acids into a plurality of test-cells, wherein each of said nucleic acids comprises a different nucleotide sequence; b) screening the plurality of test-cells for a cell exhibiting an altered phenotype, wherein the altered phenotype is due to the presence of an inhibitor. The methods may also include the steps of c) isolating the test-cell(s) exhibiting an altered phenotype; and d) isolating a candidate nucleic acid from the cell(s). The introduction (e.g. by way of retroviral vectors) and construction of suitable molecular libraries of randomized candidate nucleic acids is detailed, e.g. in WO 97/27213. The test-cells to be used in such systems are preferably devoid of hyaluronan-synthase or, alternatively, the hyaluronan-synthase is inhibited as exemplified above (antisense; iRNA, and so on). Furthermore, it is possible that the test-cells over-express one or more ABC-transporter(s) in order to mimic an arthritic phenotype. The hyaluronan-transporting phenotype may be measured as described herein e.g. introducing labelled hyaluronan or hyaluronan-derivatives, -analogues and the like into the test-cell and measuring the amount of hyaluronan (e.g. fluorescent labelled hyaluronan) before and after the introduction (and/or expression) of said library of randomized candidate nucleic acids. It is also envisaged that the library of randomized candidate nucleic acids is under the control of a regulatable promoter (described elsewhere herein) which allows a precise control of the experimental conditions.

Compounds tested positive for being capable of reducing the hyaluronan transport across a lipid bilayer are prime candidates for the direct use as a medicament or as lead compounds for the development of a medicament. Naturally, the toxicity of the compound identified and other well-known factors crucial for the applicability of the compound as a medicament will have to be tested. Methods for developing a suitable active ingredient of a pharmaceutical composition on the basis of the compound identified as a lead compound are described elsewhere in this specification.

The term “indicator compound” within the meaning of the present invention relates to hyaluronan that has been metabolically labelled in Streptococci or eukaryotic cells with [³H]glucosamine or [¹⁴C]glucose [41,42,69]; hyaluronan that has been labelled during synthesis from membranes of Streptococci or eukaryotic cells in vitro by incubation with UDP-N-acetyl-[³H]glucosamine or UDP-[¹⁴C]glucuronic acid [41,42,69]; Oligosaccharides of hyaluronan that have been radioactively labelled by reduction with NaB³H₄ [103]; hyaluronan or oligosaccharides of hyaluronan that have been biotinylated [99]; hyaluronan or oligosaccharides of hyaluronan that have been made fluorescent with rhodamine or fluorescamine [100]; hyaluronan or oligosaccharides of hyaluronan that have been iodinated [101]. In this regard, the term “oligosaccharides of hyaluronan” means 2 to 50 repeating units of disaccharides composed of glucuronic acid and N-acetylglucosamine.

It is also envisaged to employ other substances instead of hyaluronan or analogues thereof. Such “other substances” to be used as indicator substances/compounds within the meaning of the present invention are characterized by the following characteristics:
a) they are transported by an ABC-transporter which is able to transport also hyaluronan across a lipid bilayer;
b) they are detectable in an in vitro assay (i.e. they are detectably labeled or they are otherwise detectable, e.g. by way of antibody-binding; thin layer chromatography, mass spectrometry; HPLC; FPLC, or the like);
c) they do not or essentially do not interfere with the transport rate of the ABC-transporter which is measured (i.e. they do not inhibit per se the ABC-transporter).

Suitable labels are known in the art and include radioisotopes, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin. Metabolic labelling of hyaluronan or labelling in direct enzyme assay is preferable performed with carbon (¹⁴C) and tritium (³H) [97]. Hyaluronan can also be quantified by colour reactions such as the carbazol reaction [98] or by sensitive assays utilizing hyaluronan labelled with biotin [99], fluorescent groups [100], iodine[101].

In another embodiment, the present invention relates to a method for screening a compound which reduces the transport of hyaluronan mediated by (an) ABC-transporter(s), said method comprising:
(a) contacting an isolated lipid bilayer comprising at least one ABC-transporter which is capable of transporting hyaluronan with a test compound and an indicator compound;
(b) measuring the effect of the test compound on the transport of the indicator compound across the lipid bilayer; and
(c) identifying test compounds which reduce the transport of the indicator compound.

In this regard, the term “isolated lipid bilayer” relates to isolated cell-membranes or liposomes/micelles which comprise at least one ABC-transporter to be tested. Methods for the isolation of cell membranes are well-known and furthermore exemplified in the appended examples. Further, it is envisaged that the term “isolated lipid bilayer” also relates to intact but locally separated cell-(membranes), i.e. the respective part of the cell membrane is separated by patch-clamp methods or the like. In such cases (as well as in the case of liposome/micelle methods) it is necessary to introduce the indicator-compound into the cell/liposome (which has been described elsewhere herein). Alternatively, it is also envisaged that the ABC-transporter is encompassed in a so-called “Black Lipid Membrane” which allows for the specific validation of the transport of a respective test-compound across a lipid bilayer. All the above-mentioned methods are well-known to the skilled person.

In a further embodiment, the present invention relates to a method of screening for a compound which is suitable for the treatment of a disease which is associated with an excess transport of hyaluronan across a lipid bilayer, e.g. arthritis, said method comprising:
(a) contacting a cell comprising at least one ABC-transporter which is capable of transporting hyaluronan with a test compound and an indicator compound;
(b) measuring the effect of the test compound on the transport of the indicator compound across a lipid bilayer of the cell; and
(c) identifying compounds which reduce the transport of the indicator compound.

It has to be understood that such test compounds which show an effect on the hyaluronan transport (i.e. which reduce the transport activity of hyaluronan as mediated by an ABC-transporter, e.g. when compared to the same test without the addition of the respective test-compound) are regarded as compounds which are suitable for the treatment of a disease which is associated with an excess transport of hyaluronan across a lipid bilayer, e.g. arthritis. Such compounds can subsequently serve as lead compounds which e.g. can be refined as mentioned herein below.

In a further embodiment, the present invention relates to a method for screening a compound which reduces the transport of hyaluronan mediated by (an) ABC-transporter(s), said method comprising:
(a) contacting a cell comprising at least one ABC-transporter which is capable of transporting hyaluronan with a test compound and an indicator compound;
(b) measuring the effect of the test compound on the transport of the indicator compound across a lipid bilayer of the cell; and
(c) identifying compounds which reduce the transport of the indicator compound.

As mentioned elsewhere before, it is envisaged that the term “contacting a cell with an indicator-compound” means to introduce the test-compounds by suitable methods like electro-chemical-poration; lipofection; bioballistics or microinjection into the closed compartment (cell/liposome or the like).

In a preferred embodiment of the inventive methods, said screened compound specifically reduces the transport of hyaluronan mediated by said ABC-transporter. The meaning of the term “specifically reduces” was already explained herein above.

It is envisaged that the cell can be a bacterial, an insect, a fungal, or an animal cell.

The term “bacterial” is meant to include all bacteria which can be transformed or transfected with a DNA or RNA molecules for the expression of one or more ABC-transporter(s). Bacterial hosts may include gram negative as well as gram positive bacteria such as, for example, E. coli, S. typhimurium, Serratia marcescens and Bacillus subtilis. A polynucleotide sequence encoding one or more ABC-transporter(s) can be used to transform or transfect the cell using any of the techniques commonly known to those of ordinary skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989).

In one embodiment of the methods of the invention, said animal cell is a mammalian cell or a mammalian cell line.

In a preferred embodiment said mammalian cell or mammalian cell line is derived from human, horse, swine, goat, cattle, mouse or rat.

In a further preferred embodiment of the methods of the invention the cell or cell line is a chondrocyte, a fibroblast [97], sarcomas, carcinomas, smooth muscle cells, endothelial cells, endodermal cells, liver stellate cells, mesothelioma cells, melanoma cells, oligodendroglial cells, glioma cells, Schwann cells, synovial cells, myocaridal cells, trabecular-meshwork cells, cumulus cells, liver adipocytes (Ito cells), keratinocytes, epithelial cells, macrophages.

In a further embodiment of the methods of the invention, said cell is comprised in a tissue.

We analyzed the efficacy of several inhibitors in organ cultures of bovine articular cartilage that was induced to become osteoarthritic by interleukin. The inhibition of proteoglycan loss was analysed histologically by staining with safranin O. The following inhibitors were applied in different concentrations: Valspodar, Verapamil, Nicardipin, Nefidipine, Bepidril, Amiloride (see FIG. 15). FIG. 17 shows the concentration dependent inhibition of proteoglycan loss by Valspodar. FIG. 15 shows that Valspodar and Verapamil gave the best protection. Also nicardipin and nefidipin that are similar to Verapamil showed fairly good protection, whereas bepidril and amyloride were only inhibitory at high concentrations. All 6 substances are ABC-B (MDR) inhibitors [53]. The results described above indicate that inhibitors of hyaluronan export from chondrocytes can be applied to protect from proteoglycan loss in arthritis.

Bovine articular cartilage can be obtained from a local slaughter house. Human carticular is obtained from therapeutic synovectomies. Inhibition of hyaluronan transport can be measured in slices of articular cartilage cultivated as organ cultures. Hyaluronan produced in cartilage slices can be visualized histochemically by staining with labelled hyaluronan binding proteins such as the binding region of aggrecan or link protein.

Accordingly, in a preferred embodiment of the methods of the invention said tissue is cartilage tissue; tumor tissue, skin, liver, lung, blood vessels, synovia, brain, heart, glands, eye, ovary or kidney.

In a preferred embodiment of the methods of the present invention said cell or said tissue is derived from a mammalian subject preferably a human subject which suffers from a disease which is associated with an excess transport of hyaluronan across a lipid bilayer, e.g. arthritis. If this patient had a synovectomy or cartilage tissue was obtained during arthroscopy, the cartilage material can be taken into organ culture as described above and tested for recovery of cartilage production under the influence of the ABC transport inhibitors as described in the experimental section.

In another preferred embodiment of the methods of the present invention the cell comprises at least one heterologous ABC-transporter. In this context, the term “heterologous” means that the respective ABC-transporter (which is heterologous) is derived from another subject or has a different origin as the cell in question (e.g. expression of a bovine ABC-transporter in a human cell or expression of a human ABC-transporter in a bacterial/fungal/insect cell etc). Thus, it is envisaged that the cells to be used in the screening assay of the invention may comprise a heterologous polynucleotide sequence which allows the expression of said heterologous ABC-transporter.

Preferably, the polynucleotide encoding the ABC-transporter is part of a vector, e.g. a commercially available vector. Nonlimiting examples include plasmid vectors compatible with mammalian cells, such as pUC, pBluescript (Stratagene), pET (Novagen), pREP (Invitrogen), pCRTopo (Invitrogen), pcDNA3 (Invitrogen), pCEP4 (Invitrogen), pMC1 neo (Stratagene), pXT1 (Stratagene), pSG5 (Stratagene), EBO-pSV2neo, pBPV-1, pdBPVMMTneo, pRSVgpt, pRSVneo, pSV2-dhfr, pUCTag, pIZD35, pLXIN and pSIR (Clontech) and pIRES-EGFP (Clontech). Baculovirus vectors such as pBlueBac, BacPacz Baculovirus Expression System (CLONTECH), and MaxBac™ Baculovirus Expression System, insect cells and protocols (Invitrogen) are available commercially and may also be used to produce high yields of biologically active protein. (see also, Miller (1993), Curr. Op. Genet. Dev., 3, 9; O'Reilly, Baculovirus Expression Vectors: A Laboratory Manual, p. 127). In addition, prokaryotic vectors such as pcDNA2; and yeast vectors such as pYes2 are nonlimiting examples of other vectors suitable for use with the present invention. For vector modification techniques, see Sambrook and Russel (2001), loc. cit. Vectors can contain one or more replication and inheritance systems for cloning or expression, one or more markers for selection in the host, e.g., antibiotic resistance, and one or more expression cassettes.

The coding sequences inserted in the vector can be synthesized by standard methods, isolated from natural sources, or prepared as hybrids. Ligation of the coding sequences to transcriptional regulatory elements (e.g., promoters, enhancers, and/or insulators) and/or to other amino acid encoding sequences can be carried out using established methods.

Furthermore, the vectors may, in addition to the nucleic acid sequences of the invention, comprise expression control elements, allowing proper expression of the coding regions in suitable hosts. Such control elements are known to the artisan and may include a promoter, translation initiation codon, translation and insertion site or internal ribosomal entry sites (IRES) (Owens, Proc. Natl. Acad. Sci. USA 98 (2001), 1471-1476) for introducing an insert into the vector. Preferably, the nucleic acid molecule of the invention is operatively linked to said expression control sequences allowing expression in eukaryotic or prokaryotic cells. Particularly preferred are in this context control sequences which allow for correct expression in neuronal cells and/or cells derived from nervous tissue.

Control elements ensuring expression in eukaryotic and prokaryotic cells are well known to those skilled in the art. As mentioned above, they usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in for example mammalian host cells comprise the CMV-HSV thymidine kinase promoter, SV40, RSV-promoter (Rous sarcome virus), human elongation factor 1α-promoter, CMV enhancer, CaM-kinase promoter or SV40-enhancer.

For the expression in prokaryotic cells, a multitude of promoters including, for example, the tac-lac-promoter, the lacUV5 or the trp promoter, has been described. Beside elements which are responsible for the initiation of transcription such regulatory elements may also comprise transcription termination signals, such as SV40-poly-A site or the tk-poly-A site, downstream of the polynucleotide. In this context, suitable expression vectors are known in the art such as Okayama-Berg cDNA expression vector pcDV1 (Pharmacia), pRc/CMV, pcDNA1, pcDNA3 (In-Vitrogene, as used, inter alia in the appended examples), pSPORT1 (GIBCO BRL) or pGEMHE (Promega), or prokaryotic expression vectors, such as lambda gt11. An expression vector according to this invention is at least capable of directing the replication, and preferably the expression, of the nucleic acids and protein of this invention. Suitable origins of replication include, for example, the Col E1, the SV40 viral and the M 13 origins of replication. Suitable promoters include, for example, the cytomegalovirus (CMV) promoter, the iacZ promoter, the gai10 promoter and the Autographa californica multiple nuclear polyhedrosis virus (AcMNPV) polyhedral promoter. Suitable termination sequences include, for example, the bovine growth hormone, SV40, iacZ and AcMNPV polyhedral polyadenylation signals. Examples of selectable markers include neomycin, ampicillin, and hygromycin resistance and the like. Specifically-designed vectors allow the shuttling of DNA between different host cells, such as bacteria-yeast, or bacteria-animal cells, or bacteria-fungal cells, or bacteria invertebrate cells.

A method for the production of a transgenic non-human animal, preferably transgenic mouse, is also within the scope of the present invention, said method comprising introduction of a polynucleotide or vector encoding one or more ABC-transporter(s) as described herein into a germ cell, an embryonic cell, stem cell or an egg or a cell derived therefrom. The non-human animal can be used in accordance with a screening method of the invention described herein and may be a non-transgenic healthy animal, or may have a degeneration and/or a destruction of cartilage, preferably by a disorder caused by an increased hyaluronan-transport across a lipid bilayer. Such transgenic animals are well suited for, e.g., pharmacological studies of drugs in connection with a disease which is associated with an excess transport of hyaluronan across a lipid bilayer, e.g. arthritis. Production of transgenic embryos and screening of those can be performed, e.g., as described by A. L. Joyner Ed., Gene Targeting, A Practical Approach (1993), Oxford University Press. The DNA of the embryonal membranes of embryos can be analyzed using, e.g., Southern blots with an appropriate probe; see supra.

The present invention also opens up the possibility to produce e.g suitable test-systems like transgenic non-human animals with an increased level of the ABC-transporter(s) as described above and, thus, with an over-production/excess transport of hyaluronan across a lipid bilayer. Accordingly, it is possible to screen for compounds which subsequently reduce this over-expression of ABC-transporter(s) e.g. by the expression of antisense-RNA, ribozymes, of molecules which combine antisense and ribozyme functions and/or of molecules which provide for a co-suppression effect. When using the antisense approach for reduction of the amount of ABC-transporter(s) in cells, the nucleic acid molecule encoding the antisense-RNA is preferably of homologous origin with respect to the animal species used for transformation. However, it is also possible to use nucleic acid molecules which display a high degree of homology to endogenously occurring nucleic acid molecules encoding a ABC-transporter. In this case the homology is preferably higher than 80%, particularly higher than 90% and still more preferably higher than 95%. The reduction of the synthesis of a protein according to the invention in the transgenic mammalian cells can result in an alteration in, e.g., hyaluronan synthesis/transport across a lipid bilayer.

The effectiveness of a given antisense oligomer molecule in forming a heteroduplex with the target RNA may be determined by screening methods known in the art. For example, the oligomer is incubated in a cell culture expressing one or more ABC-transporter(s) as mentioned herein, and the effect on the target RNA is evaluated by monitoring the presence or absence of heteroduplex formation with the target sequence and non-target sequences using procedures known to those of skill in the art.

Accordingly, the present invention also relates to screening methods as indicated herein above, wherein the cell and/or said tissue is comprised in a non-human animal. Said non-human animal may be a vertebrate or invertebrate animal.

In this embodiment, the effect of the test compound may be assessed by observing the disease state of a non-human animal which is characterized by an increased hyaluronan transport as mediated by one or more ABC-transporter(s). Said increased hyaluronan transport may be mediated by the over-expression of one or more ABC-transporter(s) or by the stimulation of the transport of hyaluronan mediated by one or more ABC-transporter(s). With regard to the mentioned stimulation, it has to be understood that most growth factors and cytokines cause enhanced hyaluronan production in responsive target cells. Thus, if the animal suffers from an artificially induced a disease which is associated with an excess transport of hyaluronan across a lipid bilayer, e.g. arthritis (induced e.g. by over-expression or stimulation of one or more ABC-transporter(s)) prior to the administration of the test compound and the administration of the test compound (which may be repeated) results in an amelioration of the disease, then it can be concluded that the test compound is a prime candidate for the development of a medicament useful also in other mammalians, preferably humans. In addition, the compound can also inhibit the disease establishment when administered in advance.

In this context, it is envisaged that said non-human animal comprises a cell or a tissue comprising said cell as defined herein, which encodes and expresses at least one ABC-transporter which is capable of transporting hyaluronan across a lipid bilayer. Preferably, said cell or tissue comprising said cell is characterized by an over-expression of the ABC-transporter, i.e. the amount of the ABC-transporter in the cell is increased when compared with a reference cell (said over-expression can be easily quantified by methods well-known to the skilled person e.g. by antibody-binding; FACS-analysis; ELISA or the like). In one embodiment, it is envisaged that the over-expression is effected by transfecting the cell with a polynucleotide sequence which is capable of expressing one or more ABC-transporter(s) as defined herein. Corresponding methods for providing and transfecting a polynucleotide sequence encoding one or more ABC-transporter(s) are well-known to the skilled person. It is envisaged that the reference cell is for example a cell derived from the same subject but which was not transfected with a polynucleotide sequence capable of expressing one or more ABC-transporter(s).

In another embodiment, said over-expression of one or more ABC-transporter(s) can be effected by stimulating the expression and/or the hyaluronan transport capacity of one or more ABC-transporter(s) in order to simulate a disease state which can be correlated with a disease which is associated with an excess transport of hyaluronan across a lipid bilayer, e.g. arthritis. The following agents have been shown to stimulate hyaluronan transport: IL-1, TNFa, IL-12, bFGF and TGFβ1, serum, dexamethason, EGF, PDGF, GFGF, n-butyrate, phorbol esters, dibutyryl-cAMP, cAMP, insulin, hyaluronidase, follicle stimulating hormone, PGE₂, parathyroid hormone, retinoic acid. However, provided with the teaching of the present invention it is of course feasible to search/identify further substances which exert an stimulatory effect on the over-expression of one or more ABC-transporter(s).

Unlike spontaneous arthritis, experimental induction in animal makes it possible to influence the onset and course of the disease. The lesions can be studied macroscopically, histologically and biochemically, to assess the role of mediators such as cytokines, growth factors, and proteases. In the rat model spontaneous locomotor activity is rapidly, transiently, and dose-dependently decreased after iodoacetate injection into rat knees (primary response). Thereafter, only high doses (0.3 mg and 3.0 mg) lead to a secondary progressive long-term loss of spontaneous mobility on day 15, when subchondral bone is exposed. These 2 doses result in significant changes in cartilage proteoglycan concentration at day 15 and a strong inhibition of anabolism in the peripheral patellae by day 2, contrasting with the effects of lower doses (0.01, 0.03, and 0.1 mg). Thus when a sufficient dose of iodoacetate is used, this model can easily and quickly reproduce arthritis-like lesions and functional impairment in rats, similar to that observed in human disease. These parameters, as well as proteoglycan metabolism, serve as indicators for studying chondroprotective drugs, or for evaluating the ability of imaging techniques to detect and evaluate chondral lesions.

The rat model of osteoarthritis was utilized to test whether Verapamil could protect cartilage from the loss of proteoglycans [77]. Osteoarthritic damage was induced in the left knees iodoacetate into the synovial cavity. The right knees remained untreated and served as controls. Three rates were feed with normal drinking water and three with drinking water containing Verapamil. After 17 days the rats were sacrificed and the articular cartilage was analysed histologically. Acid polysaccharides were stained with alcian blue and the preparation was counterstained with nuclear fast red. FIG. 16 shows that Verapamil completely inhibited proteoglycan loss. These results indicate that inhibitors of hyaluronan export from chondrocytes can be applied to protect from proteoglycan loss in osteoarthritis/arthritis.

It will be understood that some of the screening-methods of the present invention can be carried out in vivo or ex vivo (in vitro).

The methods of the present invention also allows for the diagnosis of a subject at risk for a disease which is associated with an excess transport of hyaluronan across a lipid bilayer, e.g. arthritis or the diagnosis of a disease which is associated with an excess transport of hyaluronan across a lipid bilayer, e.g. arthritis, inter alia by the identification of an over-expression of ABC-transporter(s) and/or the identification of an increased transport of hyaluronan as mediated by ABC-transporter(s).

In accordance with this embodiment, the diagnosis can, e.g., be effected by isolating cells from an individual. Such cells can be collected from body fluids, skin, hair, biopsies and other sources. Collection and analysis of cells from bodily fluids such as blood, urine and cerebrospinal fluid is well known to the art; see for example, Rodak, “Haematology: Clinical Principles & Applications” second ed., WB Saunders Co, 2002; Brunzel, “Fundamentals of Urine and Body Fluids Analysis”, WB Saunders Co, 1994; Herndon and Brumback (Ed.), “Cerebrospinal Fluid”, Kluwer Academic Pub., 1989. In addition, methods for DNA isolation are well described in the art; see, for example, Sambrook et al., “Molecular Cloning: A Laboratory Manual”, 3^{rd} edition, Cold Spring Harbor Laboratory, 2001.

Thus, the present invention also relates to a method for identifying a subject at risk for a disease which is associated with an excess transport of hyaluronan across a lipid bilayer, e.g. arthritis comprising the steps of:
(a) analyzing the hyaluronan transport rate of a cell derived from said subject; and
(b) comparing the hyaluronan transport rate of said cell with the hyaluronan synthesis rate of a reference cell.

In a preferred embodiment said cell is a chondrocyte (for arthritis, e.g. osteoarthritis); sarcoma, carcinoma, mesothelioma cell, melanoma cell, glioma cell (for tumors); smooth muscle cells, endothelial cells (for ischemic edema formation); synovial fibroblasts for rheumatoic arthritis other inflammatory arthritis; myocaridal cells for heart infarct; fibroblasts for tissue rejection after organ transplantation and inflammation; liver adipocytes (Ito cells) for liver fibrosis; trabecular-meshwork for glaucoma.

Over the past 20 years, genetic heterogeneity has been increasingly recognized as a significant source of variation in drug response. Many scientific communications (Meyer, Ann. Rev. Pharmacol. Toxicol. 37 (1997), 269-296 and West, J. Clin. Pharmacol. 37 (1997), 635-648) have clearly shown that some drugs work better or may even be highly toxic in some patients than in others and that these variations in patient's responses to drugs can be related to molecular basis. This “pharmacogenomic” concept spots correlations between responses to drugs and genetic profiles of patient's (Marshall, Nature Biotechnology, 15 (1997), 954-957; Marshall, Nature Biotechnology, 15 (1997), 1249-1252). In this context of population variability with regard. to drug therapy, pharmacogenomics has been proposed as a tool useful in the identification and selection of patients which can respond to a particular drug without side effects. This identification/selection can be based upon molecular diagnosis of genetic polymorphisms by genotyping DNA from leukocytes in the blood of patient, for example, and characterization of disease (Bertz, Clin. Pharmacokinet. 32 (1997), 210-256; Engel, J. Chromatogra. B. Biomed. Appl. 678 (1996), 93-103). For the founders of health care, such as health maintenance organizations in the US and government public health services in many European countries, this pharmacogenomics approach can represent a way of both improving health care and reducing overheads because there is a large cost to unnecessary drugs, ineffective drugs and drugs with side effects.

In view of the disclosure content of the present invention, it will be understood that the methods of the present invention are also useful for monitoring the efficacy and/or dosing of a drug or the likelihood of a patient to respond to a drug (for example an inhibitor of the invention). Thus, in yet another embodiment the invention relates to a method for screening for a compound which is suitable for the treatment of a disease which is associated with an excess transport of hyaluronan across a lipid bilayer, e.g. arthritis in a subject, i.e. for monitoring the efficacy and/or dosing of a drug, e.g. an inhibitor as defined herein, and/or the likelihood of a patient to respond to said drug. Said method comprises contacting a cell derived from said subject which comprises at least one ABC-transporter (preferably an ABC-transporter which is able to transport hyaluronan) with a test-compound to be tested; determining/measuring the level of expression (either transcriptional or translational) of one or more ABC-transporter(s) and/or determining/measuring the level of hyaluronan transport mediated by ABC-transporter(s) across a lipid bilayer in said cell before and after administration of the respective drug. In humans, ABC-transporter activity can be monitored by Positron Emission Tomography (PET) or Single Photon Emission Computerised Tomography (SPECT) using a radiolabelled ligand tracer for ABC-transporter(s). A modulation of ABC-transporter activity, or expression levels would reflect the activity and potency of the drug. Methods and techniques required for PET analysis are well known in the art, see, for example Paans and Vaalburq, Curr. Pharmac. Design 6 (2000), 1583-1591; van Waarde, Curr. Pharmac. Design. 6 (2000), 1593-1610; Paans et al, Methods 27 (2002), 195-207; Passchier et al., Methods 27 (2002), 278-286; Laruelle et al., Methods 27 (2002), 287-299.

Hence, the present invention also concerns the pharmacogenomic selection of drugs and prodrugs for patients suffering from a disease which is associated with an excess transport of hyaluronan across a lipid bilayer, e.g. arthritis and which are possible candidates to drug therapy. The findings of the present invention provide the options of development of new drugs for the pharmacological intervention with the aim of reducing and thereby normalizing the hyaluronan transport mediated by ABC-transporter(s) The terms “reducing” and “normalizing” have already been defined elsewhere in this specification. Also a gene therapeutical approach can be envisaged with the aid of the present invention.

The present invention relates to a method of screening for a compound which is suitable for the treatment of a disease which is associated with an excess transport of hyaluronan across a lipid bilayer, e.g. arthritis in a subject, said method comprising:
(a) contacting a cell derived from said subject which comprises at least one ABC-transporter with a test compound to be tested;
(b) measuring the effect of the test compound on the transport of an indicator compound across a lipid bilayer of said cell; and
(c) identifying compounds which reduce the transport of hyaluronan across the lipid bilayer of said cell.

In order to be suitable for the treatment of arthritis, it will be acknowledged that the compound identified has to be administered subsequently in a therapeutically effective dose which is also explained in other parts of the specification. A therapeutically effective dose refers to that amount of screened compounds/inhibitors which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

In a preferred embodiment of the screening method for a compound which is suitable for the treatment of a disease which is associated with an excess transport of hyaluronan across a lipid bilayer, e.g. arthritis in a subject, said cell (which is derived from said subject) is a chondrocyte. The term “derived from a subject” as used herein means that at least one cell is isolated from said subject. Such cells can be collected from body fluids, skin, hair, biopsies and other sources. Collection and analysis of cells from bodily fluids such as blood, urine and cerebrospinal fluid is well known to the art and was already described herein above.

In another embodiment said cell is comprised in a tissue.

In another embodiment of the of screening method for a compound which is suitable for the treatment of a disease which is associated with an excess transport of hyaluronan across a lipid bilayer, e.g. arthritis in a subject, said subject is a mammalian subject.

In a preferred embodiment said mammalian subject is a human, a horse, a camel, a dog, a cat, a pig, a cow, a goat or a fowl.

In a further embodiment of the present invention said cell is contacted with a compound selected from the group consisting of:
(a) an inhibitor of a member of the ABCB (MDR)-subfamily selected from Verapamil, Valspodar (PSC833), Elacridar (GF-120918), Bericodar (VX-710), Tariquidar (XR-9576), XR-9051, S-9788, LY-335979, MS 209, R101933; OC-144-093; Quinidine, Chloripramine, Nicardipine, Nifedipine, Amlodipine, Felodipine, Manidipine, Flunarizine, Nimodipine, Pimozide, Lomerizine, Bepridil, Amiloride, Almitrine, Amiodarone, Imipramine, Clomiphene, Tamoxifen, Toremifene, Ketocanazole, Terfenadine, Chloroquine, Mepacrin, Diltiazem, Niguldipine, Prenylamine, Gallopamil, Tiapamil, Dex-Verapamil, Dipyridamole, Pimozide, Haloperidol, Chlorpromazine, Trifluoperazine, Fluphenazine, Reserpin, Clopenthixol, Flupentixol, N-acetyldaunorubicin, Vindoline, N2762-14, N276-14, N276-17, B9309-068, BIBW-22, Carvedilol, Clofazimine, Ketoconazole, Lovastatin, N-Norgallopaniil, Simvastatin, Troleandomycin, Vinblastin, Itraconazole, Econazole, Oligomycine, Cyclosporin and Rapamycin; and/or
(b) an inhibitor of a member of the ABCA subfamily selected from Glyburide, DIDS (4,4-diisothiocyanatostilbene-2,2-disulfonic acid), Bumetanide, Furosemide, Sulfobromophthalein, Diphenylamine-2-carboxylic acid and Flufenamic acid; and/or
(c) an inhibitor of a member of the human ABC-C (MRP)-subfamily selected from MK-571, Benzbromaron, PAK-104P, Probenecid, Sulfinpyrazone, Indomethacin, Merthiolate and Ethacrynic acid; and/or
(d) (an) antibody(ies) or functional fragments thereof which is(are) specifically recognizing one or more ABC-transporter(s) capable of transporting hyaluronan across a lipid bilayer; and/or
(e) (an) antisense oligomere(s), iRNA and/or siRNA directed against one or more ABC-transporter(s) capable of transporting hyaluronan across a lipid bilayer; and/or
(f) (an) aptamer(s) directed against one or more ABC-transporter(s) capable of transporting hyaluronan across a lipid bilayer.

The test-compound is in a further embodiment of the screening-methods of the present invention a small molecule or a peptide derived from an at least partially randomised peptide or aptamer library.

Furthermore, the present invention relates to methods which further comprise a step of refining the compound identified, said method comprising the steps of:
(a) identification of the binding sites of the compound and the ABC-transporter(s);
(b) molecular modelling of the binding site of the compound; and
(c) modification of the compound to improve its binding specificity for the ABC-transporter(s).

All techniques employed in the various steps of the method of the invention are conventional or can be derived by the person skilled in the art from conventional techniques without further ado. Thus, biological assays based on the herein identified nature of the polypeptides may be employed to assess the specificity or potency of the inhibitors (sometimes also referred to as drugs) wherein the increase or decrease, e.g of the hyaluronan-transport across a lipid bilayer, may be used to monitor said specificity or potency. Steps (a) and (b) can be carried out according to conventional protocols. A protocol for site directed mutagenesis is described in Ling M M, Robinson B H. (1997) Anal. Biochem. 254: 157-178. The use of homology modeling in conjunction with site-directed mutagenesis for analysis of structure-function relationships is reviewed in Szklarz and Halpert (1997) Life Sci. 61:2507-2520. For example, identification of the binding site of said drug by site-directed mutagenesis can be achieved by modifications in the primary sequence of an identified ABC-transporter which is responsible for an excess transport of hyaluronan, that affect the drug affinity; this usually allows to precisely map the binding pocket for the drug.

As regards step (b), the following protocols may be envisaged: Once the effector site for drugs has been mapped, the precise residues interacting with different parts of the drug can be identified by combination of the information obtained from mutagenesis studies (step (a)) and computer simulations of the structure of the binding site provided that the precise three-dimensional structure of the drug is known (if not, it can be predicted by computational simulation). If said drug is itself a peptide, it can be also mutated to determine which residues interact with other residues in the polypeptide of interest.

Finally, in step (c) the drug can be modified to improve its binding affinity or ist potency and specificity. If, for instance, there are electrostatic interactions between a particular residue of interest and some region of the drug molecule, the overall charge in that region can be modified to increase that particular interaction. Identification of binding sites may be assisted by computer programs. Thus, appropriate computer programs can be used for the identification of interactive sites of a putative inhibitor and the polypeptide by computer assisted searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. Modifications of the drug can be produced, for example, by peptidomimetics and other inhibitors can also be identified by the synthesis of peptidomimetic combinatorial libraries through successive chemical modification and testing the resulting compounds. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715.

In accordance with the above, in a preferred embodiment of the method of the invention said compound is further refined by peptidomimetics.

The invention furthermore relates in a further preferred embodiment to a method of modifying a compound identified or refined by the method as described herein above as a lead compound to achieve (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmakinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophylic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetates, or (xiv) N-acetylation to amides, phenylcarbamates, or (xv) synthesis of Mannich bases, imines, or (xvi) transformation of ketones or aldehydes to Schiff's bases, oximes, acetates, ketales, enolesters, oxazolidines, thiozolidines or combinations thereof.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

The invention moreover relates in a further preferred embodiment to a method further comprising producing a pharmaceutical composition comprising formulating the compound identified, refined or modified by the method of any of the preceding claims with a pharmaceutically active carrier and/or diluent.

Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10⁶ to 10¹² copies of the DNA molecule. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use.

The present invention also relates to a method for manufacturing a pharmaceutical composition comprising the steps of any one of the aforementioned screening methods and the step of formulating the compound screened in a pharmaceutically acceptable form. In this regard, the term “in a pharmaceutically acceptable form” refers to the formulation of the compounds screened with a pharmaceutically acceptable carrier and/or diluent. Examples of suitable pharmaceutical carriers are well known in the art and have already been described herein above.

The present invention also relates to a method of preventing, ameliorating and/or treating the symptoms of a disease which is associated with an excess transport of hyaluronan across a lipid bilayer, e.g. arthritis in a subject comprising administering at least one inhibitor as defined herein/screened by the methods disclosed herein of at least one ABC-transporter capable of transporting hyaluronan across a lipid bilayer to the subject such that the disease which is associated with an excess transport of hyaluronan across a lipid bilayer, e.g. arthritis is prevented, ameliorated and/or treated.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease. The present invention is directed towards treating patients with medical conditions relating to a disease which is associated with an excess transport of hyaluronan across a lipid bilayer, e.g. arthritis. Accordingly, a treatment of the invention would involve preventing, inhibiting or relieving any medical condition related to a disease which is associated with an excess transport of hyaluronan across a lipid bilayer, e.g. arthritis. As mentioned elsewhere before, said arthritis is e.g. characterized by a degeneration and/or a destruction of cartilage.

In a preferred embodiment said arthritis is osteoarthritis, (juvenile) chronic arthritis, rheumatoid arthritis, psoriatic arthritis, A. mutilans, septic arthritis, infectious arthritis and/or reactive arthritis.

In a preferred embodiment, said at least one ABC-transporter is MRP5 (ABCC5), ABCC11 and/or ABCC12. In a more preferred embodiment said at least one ABC-transporter is MRP5 (ABCC5).

In another preferred embodiment the inhibitor is Zaprinast® which is used for the preventing, ameliorating and/or treating arthritis, preferably rheumatoid arthritis or osteoarthritis.

In another preferred embodiment said inhibitor is selected from Elacridar (GF-120918), Valspodar (PSC-833), Bericodar (VX-710), Tariquidar (XR-9576), S-9788, Ly-335979, OC-144-093 and/or Lysodren® and said disease is osteoarthritis.

In the context of the present invention the term “subject” means an individual in need of a treatment of an affective disorder. Preferably, the subject is a mammalian, particularly preferred a human, a horse, a camel, a dog, a cat, a pig, a cow, a goat or a fowl.

The term “administered” means administration of a therapeutically effective dose of the inhibitors and/or test-compounds as disclosed herein. By “therapeutically effective amount” is meant a dose that produces the effects for which it is administered. The exact dose will depend on the purpose of the treatment, and will be ascertainable by one skilled in the art using known techniques.

The methods are applicable to both human therapy and veterinary applications. The compounds described herein having the desired therapeutic activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt %. The agents maybe administered alone or in combination with other treatments, i.e. it is also within the scope of the present invention to combine for example one of the already known drugs/treatments for arthritis (e.g. the injection of hyaluronan) with one or more of the inhibitors/test-compounds as defined herein.

The administration of the pharmaceutical composition can be done in a variety of ways as discussed above, including, but not limited to, orally, subcutaneously, intravenously, intra-arterial, intranodal, intramedullary, intrathecal, intraventricular, intranasally, intrabronchial, transdermally, intranodally, intrarectally, intraperitoneally, intramuscularly, intrapulmonary, vaginally, rectally, or intraocularly. In some instances, for example, in the treatment of wounds and inflammation, the candidate agents may be directly applied as a solution dry spray.

Drugs or pro-drugs after their in vivo administration are metabolized in order to be eliminated either by excretion or by metabolism to one or more active or inactive metabolites (Meyer, J. Pharmacokinet. Biopharm. 24 (1996), 449-459). Thus, rather than using the actual compound or drug identified and obtained in accordance with the methods of the present invention a corresponding formulation as a pro-drug can be used which is converted into its active in the patient. Precautionary measures that may be taken for the application of pro-drugs and drugs are described in the literature; see, for review, Ozama, J. Toxicol. Sci. 21 (1996), 323-329.

It is also envisaged that the inhibitors/test-compounds of the present invention are employed in co-therapy approaches, i.e. in co-administration with other medicaments or drugs, for example other drugs for preventing, treating or ameliorating a disease which is associated with an excess transport of hyaluronan across a lipid bilayer, e.g. arthritis.

Another aspect of the present invention is the administration of an inhibitor which leads to a reduction of the expression of a nucleic acid encoding an ABC-transporter (as defined herein) in the cells or comprising a nucleic acid molecule the expression of which in cells or the administration of which to cells leads to a reduction of the expression of a nucleic acid encoding an ABC-transporter (as defined herein) in the cells. Said inhibitor may be useful for treating individuals having an increased amount of the ABC-transporter or expression level as described hereinabove.

Preferably, the above-mentioned inhibitor is an antisense, a ribozyme, a co-suppressive nucleic acid, iRNA or siRNA.

An siRNA approach is, for example, dislosed in Elbashir ((2001), Nature 411, 494-498)). It is also envisaged in accordance with this invention that for example short hairpin RNAs (shRNAs) are employed in accordance with this invention as pharmaceutical composition. The shRNA approach for gene silencing is well known in the art and may comprise the use of st (small temporal) RNAs; see, inter alia, Paddison (2002) Genes Dev. 16, 948-958.

As mentioned above, approaches for gene silencing are known in the art and comprise “RNA”-approaches like RNAi or siRNA. Successful use of such approaches has been shown in Paddison (2002) loc. cit., Elbashir (2002) Methods 26, 199-213; Novina (2002) Mat. Med. Jun. 3, 2002; Donze (2002) Nucl. Acids Res. 30, e46; Paul (2002) Nat. Biotech 20, 505-508; Lee (2002) Nat. Biotech. 20, 500-505; Miyagashi (2002) Nat. Biotech. 20, 497-500; Yu (2002) PNAS 99, 6047-6052 or Brummelkamp (2002), Science 296, 550-553. These approaches may be vector-based, e.g. the pSUPER vector, or RNA polIII vectors may be employed as illustrated, inter alia, in Yu (2002) loc. cit.; Miyagishi (2002) loc. cit. or Brummelkamp (2002) loc. cit.

A compound which leads to a reduction of the expression of an ABC-transporter gene in question may, e.g., be a compound which acts on the regulatory region of the gene and thereby reduces the level of transcription. Such compounds can be identified by methods as described herein.

As referred to several times in this specification, in a most preferred embodiment of the methods and uses specified herein before, said ABC-transporter is MRP5 (ABCC5) having a sequence as deposited under Accession numbers NM 005688, [gi:5032100] or NM 018672-[gi:17865629], ABCC11 having a sequence as deposited under NM033151 and/or ABCC12 having a sequence as deposited under NM033226. In a most preferred embodiment it is MRP5.

MRP5 is further described in WO2002072825, WO2002069950, JP2002223759, JP2002218985, and WO2002003993. The embodiments disclosed in these applications in connection with MRP5 and its uses and methods based on MRP5 are herewith incorporated by reference.

This disclosure may best be understood in conjunction with the accompanying drawings, incorporated herein by references. Furthermore, a better understanding of the present invention and of its many advantages will be had from the following examples, given by way of illustration and are not intended as limiting.

### THE FIGURES SHOW

FIG. 1 Classification of human ABC transporters and their inhibitors.

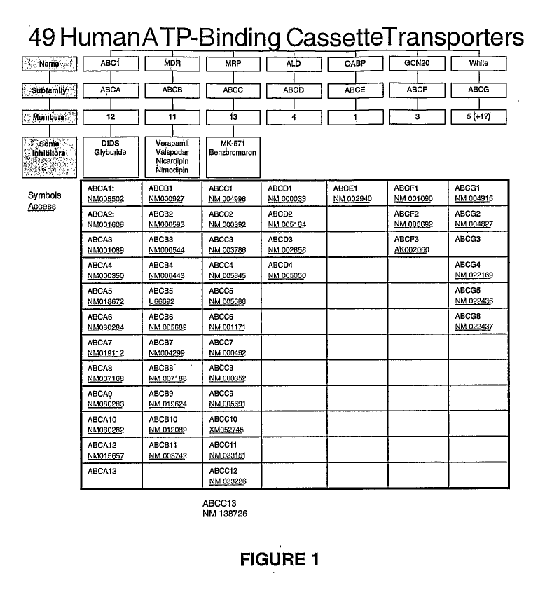

FIG. 2 Kinetics of hyaluronan production by intact streptococci: Group A Streptococcus pyogenes M49 (strain CS101pGhost9:ISS1) (), the ABC transporter mutant (□) and the rescued transfectant (▪) were grown to the exponential growth phase, harvested and incubated with [³H]glucosamine for determination of hyaluronan synthesis in intact. The amount of total [³H]hyaluronan in the culture supernatant was determined at the times indicated.

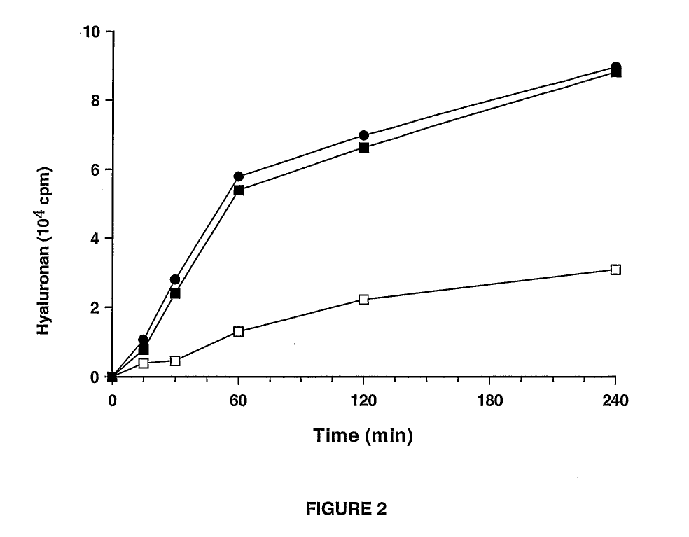

FIG. 3 Hyaluronan synthase activity and capsule production.

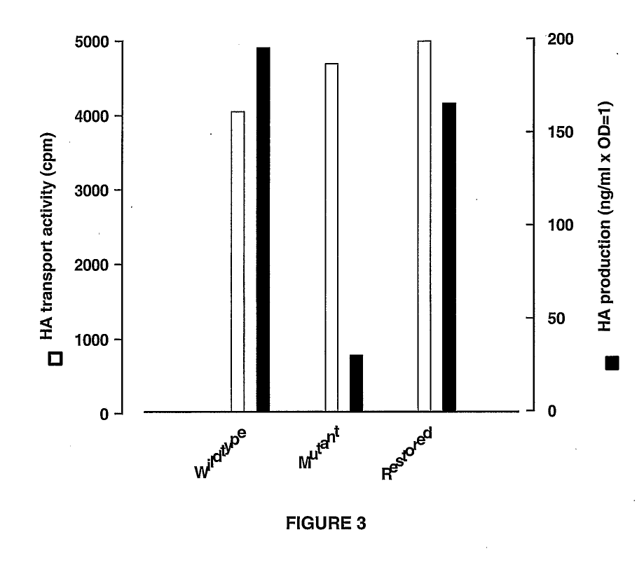

FIG. 4 Arrangement of genes for hyaluronan synthesis and export: This chromosomal segment from the known sequence of Streptococcus pyogenes M1 (strain SF370) from the data base at the University of Oklahoma Advanced Center for Genome Technology covers the loci for hyaluronan synthesis (hasA, hasB and hasc) and for the ABC transporter (haxA, haxB, haxC and haxD). ISS1, insertion sequence; Erm, erythromycin resistance; primer 1, haxupSacI; primer 2, haxdownPstI; primer 3, pGhost5SK; primer 4, pGhost5KS; primer 5, ISpGhost9P7; primer 6, ISpGhost9P8.

FIG. 5 The phylogenetic relationship of the streptococcal hyaluronan transporters Spy2194 (Hax A) and Spy2195 (Hax B) with 2 members of the human ABCA, ABCB and 12 members of the ABCC subfamily were aligned with the CLUSTALW program. The designations are given as names and symbols. The distance measure is given in substitutions per amino acid.

FIG. 6 Concentration dependent inhibition of growth and hyaluronan production of human skin fibroblasts by Verapamil and Valspodar.

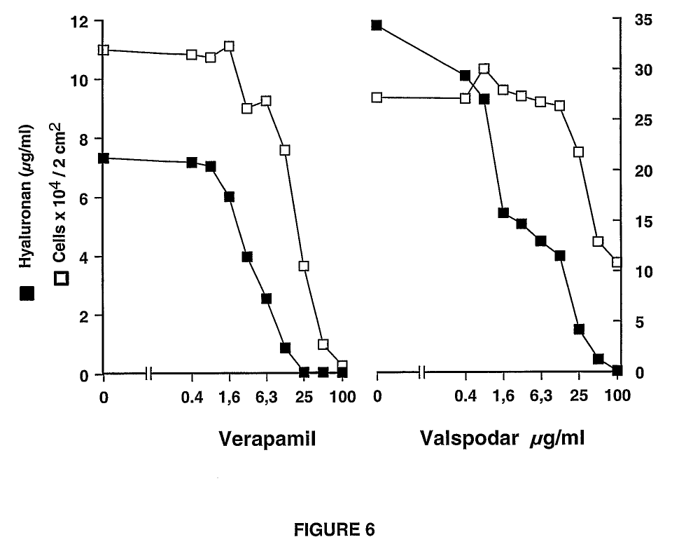

FIG. 7 Concentration dependent inhibition of growth and hyaluronan production of human synovial fibroblasts by Verapamil and Valspodar.

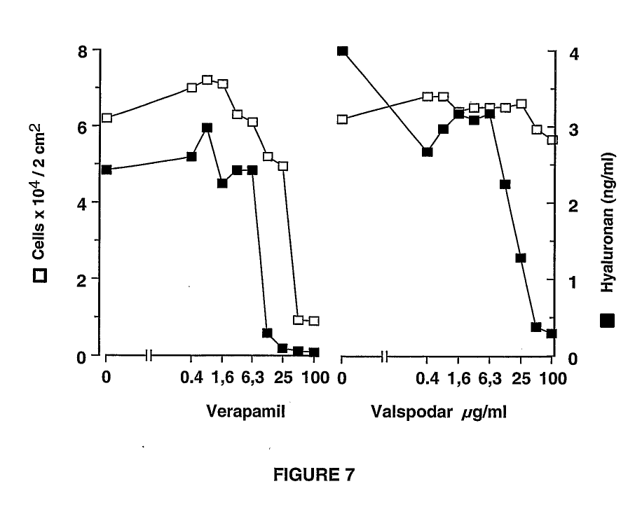

FIG. 8 Concentration dependent inhibition of hyaluronan synthase activity in membranes form human skin fibroblasts by Verapamil and Valspodar.

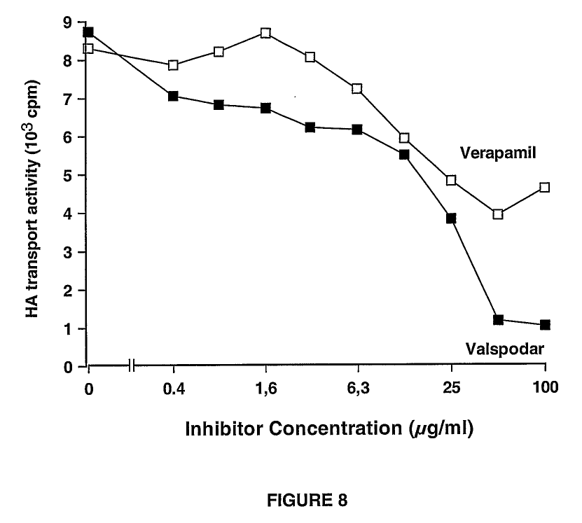

FIG. 9 Concentration dependent inhibition of hyaluronan production of the human colon carcinoma cell lines HT29 and HT29 mdr by Verapamil.

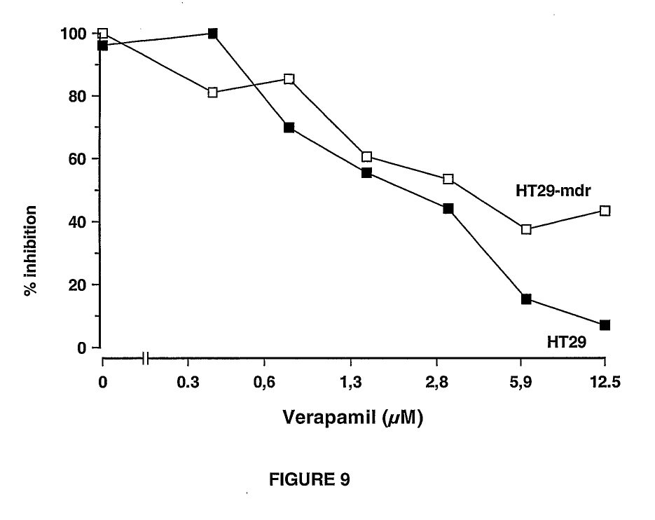

FIG. 10 Concentration dependent inhibition of hyaluronan synthase activity of membranes from the human colon carcinoma cell lines HT29 and HT29 mdr by Verapamil.

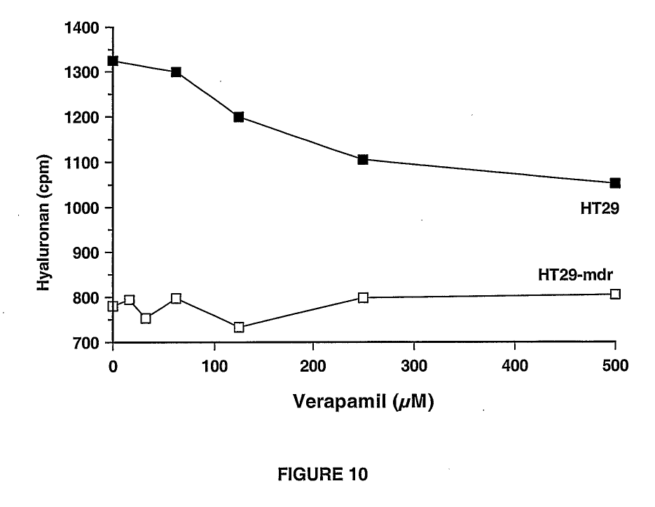

FIG. 11 Concentration dependent inhibition of hyaluronan production of a human fibroblast cell line by glyburide and Valspodar.

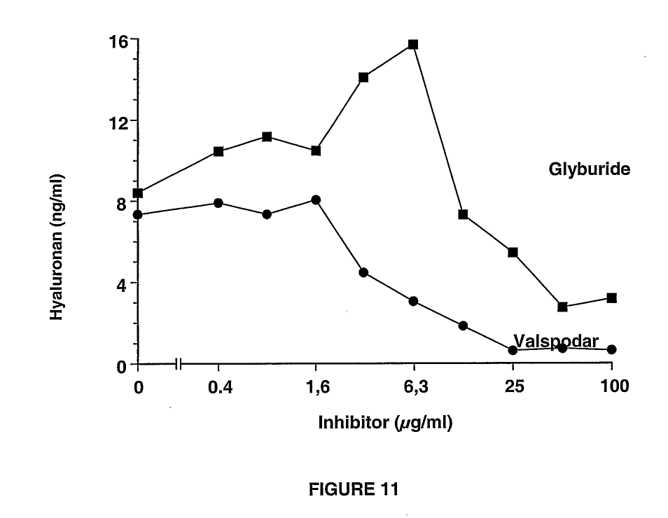

FIG. 12 Concentration dependent inhibition of hyaluronan synthase activity in membranes from a human fibroblast cell line by Valspodar, benzbromarone and MK-571.

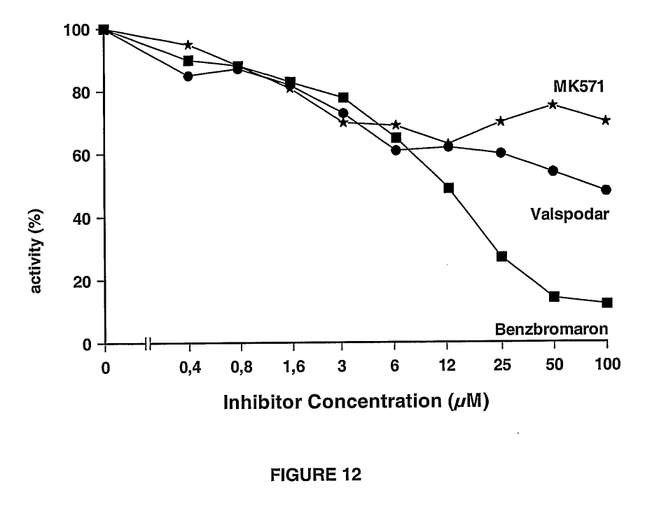

FIG. 13 Concentration dependent inhibition of hyaluronan production of a human chondrocyte cell line by Valspodar.

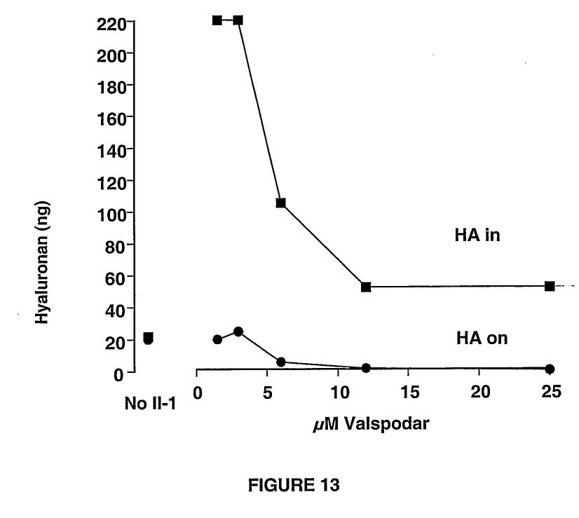

FIG. 14 Effect of Valspodar on proteoglycan production of a human chondrocyte cell line.

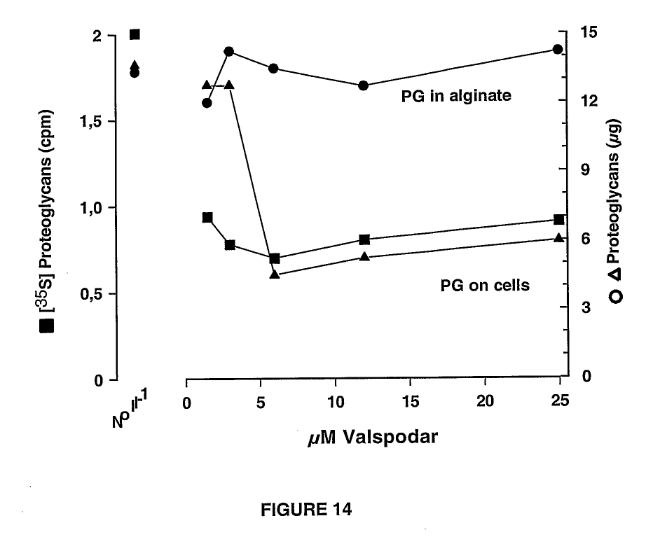

FIG. 15 Inhibition of proteoglycan loss from osteoarthritic bovine cartilage by inhibitors of ABC-transporters.

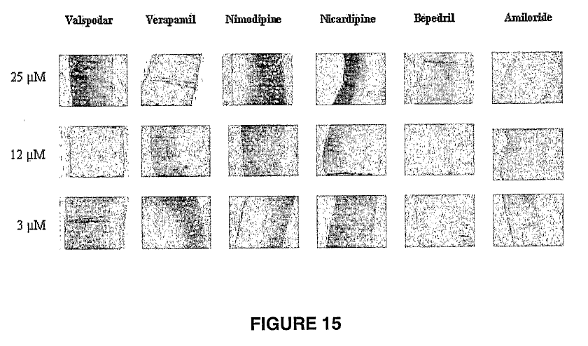

FIG. 16 Protection from proteoglycan loss from osteoarthritic cartilage in rat knee joints.

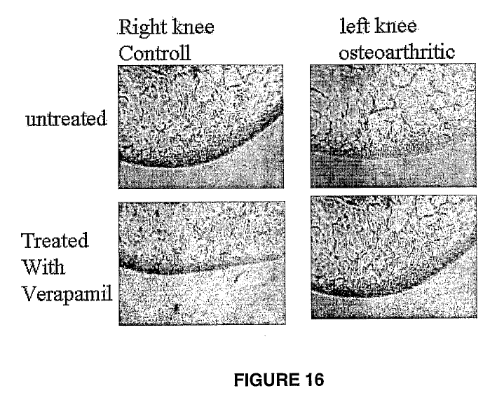

FIG. 17 Concentration dependent inhibition of proteoglycan loss from osteoarthritic bovine cartilage by Valspodar.

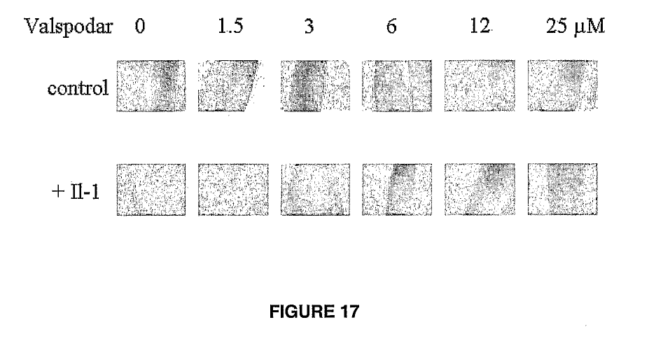

FIG. 18 Effect of ABC transporter inhibitors on the hyaluronan synthase activity (▪), hyaluronan production (□), and proliferation () of human skin fibroblasts.

FIG. 19 Effect of Verapamil and Valspodar on hyaluronan production (□) and proliferation () of human synovial fibroblasts.

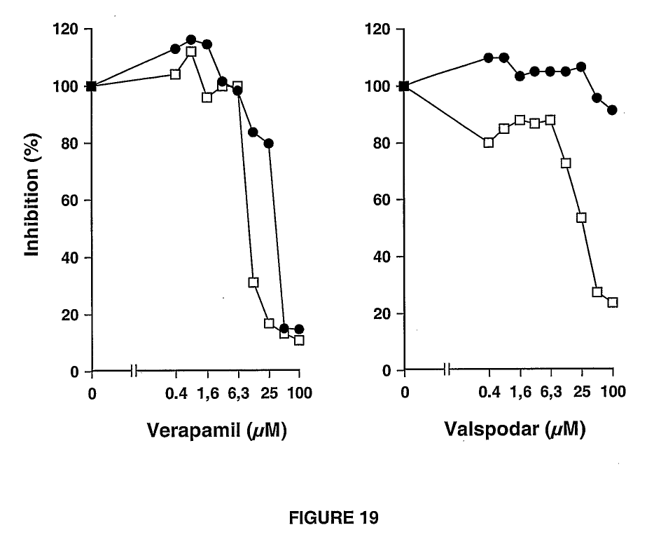

FIG. 20 Inhibition of Hyaluronan export by MRP5-RNAi

FIG. 21 Delayed treatment of osteoarthritis with Verapamil

FIG. 22 Export of fluorescein diacetate from HEK-MRP5

FIG. 23 Export of high molecular weight hyaluronan and hyaluronan oligosaccharides

FIG. 24 Export of fluorescein diacetate from human embryonic kidney (HEK) cells in the absence and presence of hyaluronan oligosaccharides s

FIG. 25 Inhibition of hyaluronan export by benzbromaron

FIG. 26 Concentration dependent inhibition of proteoglycan loss by verapamil or zaprinast

FIG. 27 Protection from collagen loss by zaprinast

### EXAMPLES

The following examples illustrate the invention. These examples should not be construed as to limit the scope of this invention. The examples are included for purposes of illustration and the present invention is limited only by the claims.

### Cells and Cell Culture

Fibroblasts were grown in suspension culture in Dulbecco's modified Eagles medium supplemented with streptomycin/penicillin (100 units of each/ml), kanamycin (100 units/ml) and 10% foetal calf serum. The human colon carcinoma cell lines HT29 and HT29-mdr were from Dr. U. Schumacher (Universitätsklinikum Hamburg) [72]. Synovial fibroblasts were extracts from synovial membranes obtained from therapeutic synovectomies. A temperature sensitive human chondrocyte cell line (tsT/AC62) was obtained from Dr. M. Goldring, Boston (REF).

### General Methods

Isolation of membranes and the determination of hyaluronan synthase activity was performed as described previously [86]. The concentration of hyaluronan in the cell culture medium was determined by an ELISA [74;87]. The proteoglycan concentration was measured by a colour reaction [75], and sulfate incorporation into proteoglycans was determined by radioactivity [76].

### Example 1

### Hyaluronan Synthesis and Export in Streptococci

### Materials

Dry media (Todd-Hewitt, T H) and media components were from GIBCO BRL. Sheep blood was from Oxoid. [³H]GlcN (specific activity 18.5 Ci/mMole) was from Amersham International, UDP-[¹⁴C]GlcA (specific activity 0.3 Ci/mMole) and UDP-[³H]GlcNac (specific activity 34.8 Ci/mMol) were from NEN. The DEAD/LIVE BacLight Bacterial Viability Kit was from Molecular Probes, Inc. All other chemicals were purchased from Sigma except stated otherwise. Restriction and other DNA modifying enzymes were from New England Biolabs. Oligonucleotide primers were synthesized by MWG Biotech.

### Bacterial Strains and Plasmids

Group A Streptococcus pyogenes M49 (strain CS101) was from A. Podbielski. Thermosensitive pGhost9:ISS1 vector containing the ISS1 insertion sequence from Lactococcus lactis and an erythromycin resistance marker was obtained from E. Maguin [64]. E coli EC101 was provided by K. Leenhout [78]. pAT28 vector with a spectinomycin resistance marker was available from P. Courvalin [79].

### General Methods

Streptococci were grown on Todd-Hewitt agar supplemented with 3% sheep blood (Oxoid), in Todd-Hewitt medium (TH) or in TH supplemented with 0,5% yeast (THY) at 37° C. CS101 mutant strain containing pGhost9:ISS1 vector was subcultured in medium supplemented with erythromycin (5 mg/l) at 37° C. Standard recombinant DNA techniques for nucleic acid preparation and analysis were performed as described [80]. DNA restriction fragments were isolated from agarose gels with the QIAquick Gel Extraction Kit (Qiagen) according to the manufacturer's instructions. Electrotransformation of E. coli was performed by the method of Dower et al. [81] with a BioRad Gene Pulser (BioRad Laboratories). Genomic streptococcal DNA was isolated as described previously [82]. Alternatively to phenol/chloroform extraction, DNeasy Tissue Kit (Qiagen) was employed after treating the cells with pronase. Plasmids were sequenced on an ABI 310 automated DNA sequencer using the ABI PRISM® Big Dye terminator cycle sequencing kit (PE Applied Biosystems).

### Electroporation of Streptococci

Streptococci were transformed by electroporation as described [83] with some modifications. Briefly, 5 ml overnight culture in THY supplemented with 20 mM glycin and 5% serum was diluted 20-fold in the same medium and grown to an OD₆₀₀ of 0,3. After addition of 20 mg hyaluronidase the bacteria were incubated at 37° C. for further 15 min and cooled on ice for an hour. The cells were harvested by centrifugation at 2,000 g and 4° C. for 10 min and washed twice in cold electroporation buffer (EPM, 272 mM glucose, 1 mM MgCl₂, pH 6,5/NaOH) and finally resuspended in 0,5 ml of cold EPM. 2 μg plasmid DNA was mixed with 100 μl of the cell suspension and electroporation was carried out at 1400 V, 25 μF and 200 in a Gene Pulser (Bio-Rad Laboratories). The cells were quickly transferred into 5 ml THY supplemented with 5% serum, incubated for 2 to 3 hours at 37° C. and plated onto selective agar plates.

Construction of S. pyogenes Mutant Library

The Streptococcus pyogenes mutant library was constructed by chromosomal insertion of the thermosensitive pGhost9:ISS1 vector [64]. Bacteria were transformed by electroporation with 1 μg of purified plasmid DNA. Selection of plasmid containing strains was performed on blood agar plates supplemented with erythromycin (5 mg/l) at a temperature of 30° C., allowing replication of the vector. To generate chromosomal integration of the plasmid, one of the isolates was grown overnight in THB (Todd Hewitt Broth, Oxoid) supplemented with erythromycin (5 mg/l) at 30° C. The saturated culture was diluted 1:100 in fresh THB medium without antibiotic pressure and incubated for 3 h at 30° C. To reduce the plasmid copy number per bacterial cell, the culture was transferred to a 38° C. water bath and incubated for another 2 h. Samples were diluted with fresh THB medium and plated on erythromycin containing blood agar plates (1 mg/l). Mutants were selected for further studies after overnight growth at nonpermissive temperature (above 37° C.).

### Determination of the ISS1 Insertion Site

To identify the chromosomal insertion site of the ISS1 insertion sequence, 2 μg of total genomic DNA of the pGhost9:ISS1 mutants was digested with EcoRI or HindIII, extracted with phenol/chloroform and ethanol precipitated. The digested DNA was diluted to a final concentration of 0.5 μg/ml and ligated with the T4 DNA ligase under conditions as described [84].The obtained plasmid mixture was transfected into E. coli EC101 [78]. Erythromycin-resistant E. coli clones containing pGhost9:ISS1 plasmid with genomic streptococcal DNA flanking the insertion site of the plasmid were selected on LB agar with erythromycin (150 mg/l). The genomic DNA contained in the recovered plasmids was sequenced with primers annealing to pGhost9:ISS1 vector sequences. To sequence the plasmids obtained after EcoRI digestion we used primers pGhost5SK and ISpGhost9P8. Primers pGhost5KS and ISpGhost9P7 were used to sequence plasmids obtained after HindIII digestion.

### Cloning of the hax Locus

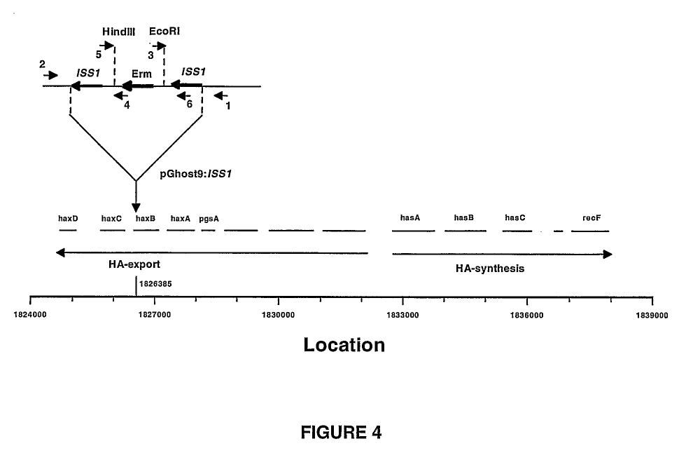

For complementation studies we cloned the hax locus into pAT28 plasmid by PCR of the genomic DNA of the wild type strain with primers haxupSacI and haxdownPstI (for primer sequences and locations see FIG. 4) and subsequent restriction digestion with Sacd and PstI and ligation into the pAT28 plasmid. After amplification in E. coli the construct was electroporated into S. pyogenes. Successful transfer of the plasmid was confirmed by plasmid preparation and subsequent PCR amplification of the cloned fragment with the primers that were used to clone the hax locus.

### Determination of Bacterial Hyaluronan Synthase Activity

Overnight cultures of streptococci were diluted at a ratio of 1:10 with fresh media and incubated at 37° C. until an exponential growth phase. The bacteria (10 ml) were sedimented at 2,000 g for 10 min, washed twice with 50 mM Tris-malonate (pH 7,0) and suspended in 2 ml 50 mM Tris-malonate (pH 7,0), 1 mM dithiothreitol, 10 mM MgCl₂, 0.15 M NaCl. The cells were lysed by ultrasonication and ultracentrifuged at 50,000 g and 4° C. for 15 min. The membrane pellet was resuspended in 250 μl of the same buffer by short ultrasonication. To 50 μl of the membrane suspension 25 μl of the substrate for hyaluronan synthesis (160_M UDP-GlcNac and 8_M UDP-[¹⁴C]GlcA (specific activity 320 mCi/mmol, 1 mM dithiothreitol, 10 mM MgCl₂, 0.15 M NaCl) and incubated for 1 h at 37° C. A solution (10 μl) of 10% SDS was added to inactivate the synthase and the mixture was applied to descending paper chromatography on Whatman MM paper for 18 h in 1 M ammonium acetate, pH 5.5/ethanol (13:7). The origin was cut out and radioactivity determined.

### Measurement of Hyaluronan Release by Intact Bacteria

The rate of hyaluronan synthesis by intact bacteria was measured by incorporation of radioactivity into hyaluronan using [³H]glucosamine in the presence of excess non-radioactive UDP-N-acetyl-glucosamine. Overnight cultures of Streptococcus pyogenes were diluted at a ratio of 1:10 with fresh media and incubated at 37° C. for three hours to reach exponential growth. Aliquots of 1 ml were sedimented for 1 min at 10,000 g, suspended in 0.5 ml of fresh medium, mixed with 10 μl of a 100 μg/μl solution of UDP-GlcNac and 25 μl of [³H]glucosamine and incubated at 37° C. After different time periods the suspension was again centrifuged for 1 min at 10,000 g. The supernatant was subjected to the descending paper chromatography as described above.

### Determination of the Hyaluronan Capsule

The capsule of the streptococcal cells was determined by the method of Schrager et al. [85] with slight modifications. Cells from exponentially growing streptococci were wasched twice with water and suspended in 0.5 ml of water. The capsule was released by shaking with 1 ml of chloroform. After centrifugation the hyaluronan content in the water phase was determined by addition of 2 ml of a solution of 20 mg of stains-all (1-ethyl-2-[1-ethylnaphto-[1,2-d]thiazolin-2-ylidene)-2-methylpropenol]naphto-[1,2-d]thiazolium bromide) and 60 μl of acetic acid in 100 ml of dimethylformide. The absorbance was read at 640 nm and compared to a standard curve of known hyaluronan concentrations.

### Bacterial Vital Stain

Dead and viable cells were differentiated by a DEAD/LIVE BacLight Bacterial Viability Kit. Shortly, 2 ml of a streptococcal culture was centrifuged and washed twice with 50 mM Tris-HCl, 0.9% NaCl pH 7.3 (TBS). The final pellet was resuspended in 3 ml TBS. The suspension (50 μl) was mixed with 1,5 p, staining solution and incubated for 15 min at room temperature in the dark. The staining solution was prepared by mixing 1 μl 1,67 mM SYTO9, 1 μl, 20 mM propidium iodide and 98 μl water. Fluorescence spectroscopy of the samples was carried out according to the manufacturer's instructions.

### Example 2

### Determination of the Effect of ABC Transport Inhibitors on Cell Proliferation and Hyaluronan Production

Cells were seeded in 24 multiwell dished with a diameter of 2 cm² at a density of 2×10⁴ cells/cm² in Dulbecco's media containing 10% foetal bovine serum and increasing concentrations of inhibitors. After incubation for 3 days at 37° C. the cells were trypsinized and counted. The concentration of hyaluronan was determined in the culture medium by an ELISA assay [87].

### Example 3

### Determination of the Effect of ABC Transport Inhibitors on the Activity of the Hyaluronan Synthase in Membranes

For determination of the concentration dependent inhibition of inhibitors on the hyaluronan synthase, cells were grown in stationary flasks (180 cm²) to near confluency. Four hours before harvest the cells were again stimulated by addition of 5% foetal bovine serum, washed with cold PBS and scrapped off with the aid of a rubber policemen. The cell suspension was collected in 30 ml of PBS and disrupted by nitrogen cavitation in a Parr bomb (15 min at 900 psi). The particular fraction was sedimented by ultracentrifugation (20 min at 100.000 g). The sediment was suspended in 1 ml of a solution of 160 μM UDP-GlcNac and 8 μM UDP-[¹⁴C]GlcA (specific activity 320 mCi/mmol, 1 mM dithiothreitol, 10 mM MgCl₂, 0.15 M NaCl. Aliquots (100 μl) were supplemented with increasing concentrations of inhibitors and incubated for 4 hours at 37° C. The hyaluronan synthase was inactivated by addition of 10 μl of 10% SDS. The suspension was subjected to descending paper chromatography for 20 hours on Whatman MM in 1 M ammonium acetate, pH 5.5/ethanol (13:7). The origin was cut out and their radioactivity radioactivity was determined.

### Example 4

### Inhibition of Hyaluronan Synthesis by Valspodar in Human Chondrocytes

Human chondrocytes were grown in RPMI media at 32° C. to near confluency and harvested by trypsinization as described [73]. The cells were suspended in an alginate solution (1.2% in 0.9% NaCl) at a cell density of 4×10⁶ cells/ml and pressed though a 22G syringe dropwise (3 drops) into a solution of 200 μl of 55 mM Na-citrate in 0.9% NaCl, pH 6.05 that had been added in the wells of a microtiter plate. This treatment leads to the formation of alginate beads containing chondrocytes. The beads in the wells of the microtiter plate were washed with PBS and incubated with RPMI-media for 5 days at 39° C. The media were than replaced with fresh media with and without interleukin-1 3 (200 μg/ml) and increasing concentrations of Valspodar and incubated for another 3 days at 39° C. The media were withdrawn, centriguded for 5 min at 2000 g and frozen until the hyaluronan assay was performed. The beads were washed with PBS and solubilzed with a solution of 125 μl 55 mM Na-citrate in 0.9% NaCl, pH 6.05 for 10 min at 37° C. The cells were sedimented at 2000 g for 5 min and suspended in 125 p, of a solution of 20 μg/ml Papain in 0.1 M Na-acetat pH 5.53, 50 mM EDTA, 0.9% NaCl, 5 mM cysteine and incubated for 20 hours at 37° C. The supernatants were supplemented with 12.5 μl of 200 μg/ml papain in 0.1 M Na-acetat pH 5.53, 50 mM EDTA, 0.9% NaCl, 5 mM cysteine and also incubated for 20 hours at 37° C. Aliquotes of these solutions were taken for determination of the hyaluronan concentration by an Elisa assay.

### Example 5

### Effect of Valspodar on proteoglycan synthesis in human chondrocytes

Human chondrocyten were grown as described above. Aliquotes were taken for the determination of the proteoglycan concentration using a colour reaction as described [75]. For measurement of the proteoglycan synthesis rate the chondrocytes in the wells of the microtiter plates were supplemented with 12.5 μl [³⁵S]SO₄²⁻ (0.5 mCi/ml) 20 hours before harvest. Aliquotes (20 μl) were used for the determination of radioactivity incorporated into [³⁵S]Proteoglycans as described [76].

### Example 6

### Determination of the Inhibitory Effects of Drugs on Proteoglycan Loss Osteoarthritic Bovine Cartilage

A bovine knee was obtained from a local slaughter and slices of cartilage (0.5 cm²) were were incubated in 1 ml of RPMI media containing 10% of foetal bovine serum in the presence and absence of interleukin-1β (5 ng/ml) and increasing concentrations of Valspodar for 3 days at 37° C. The tissues were stained histologically with fast green and safranin O. This stains proteoglycans red and other material greyish green.

### Example 7

### Treatment of Osteoarthritic Rats with VERAPAMIL

The animal trials were performed in accordance with the guidelines of the local ethical committee. Osteoarthritic damage was induced in the left knees of 6 Wistar rats by injection of a solution of 50 μl of 10 mM iodoacetate into the synovial cavity. The right knees remained untreated and served as controls. Three rates were feed with normal drinking water and three with drinking water containing 0.75 mg/ml of Verapamil. This concentration has been used previously [88]. After 17 days the rats were sacrificed and the articular cartilage was analysed histologically. Acid polysaccharides were stained with alcian blue and the preparation was counterstained with nuclear fast red.

### Example 8

### Specific Screening Assay for the Hyaluronan Transporter

The specific screening assay for the hyaluronan transporter is based on the extrusion of labelled hyaluronan oligosaccharides from intact cells in monolayer culture. For this assay the labelled oligosaccharides have to be introduced into the cytosol of cells. Because they will normally not transverse the plasma membranes, they are introduced by osmotic lysis of pinocytotic vesicles according to a method that has already successfully been applied for the introduction of periodate oxidized nucleotide sugars [25]. Alternatively, they can be introduced with the aid of cationic lipid formulations such as lipofectamine or lipofectin according to procedures that are widely used to introduce nucleic acids into living cells [116].

### Experimental

Hyaluronan oligosaccharides are prepared from commercially available hyaluronan by digestion with hyaluronidase and sized fractionation by gel filtration as described [102]. Appropriate oligosaccharide fractions having a length between 2 and 50 disaccharide units are labelled by incorporation of a biotin, radioactivity, or a fluorescent probe. These methods are routine published procedures [87,99-101,103]. The cells are seeded into multiwell microtiter plates to a density of at least 4×10⁴ cells/cm². When the cells are attached to the plastic surface after a few hours, they are washed with phosphate buffered saline and incubated with the labelled hyaluronan dissolved in medium for osmotic lysis of inocytotic vesicles (growth medium such as Dulbeccos medium containing 1 M sucrose, 50% poly(ethylene glycol)-1000) for at least 5 min up to several hours at 37° C. During this time the cells will pinocytose this hyperosmotic medium and the labelled hyaluronan. The above medium is substituted by a mixture of Dulbeccos medium and water (3:2) for 2 min. This causes the intracellular pinocytotic vesicles to lyse and to liberate the contents into the cytosol without damaging the cells. The cells can be subjected to this incubation sequence several times. The cells are washed thoroughly several times with phosphate buffered saline or growth medium to remove extracellular labelled hyaluronan and are then ready for the assay. They are incubated in growth medium containing the compound to be tested in different concentrations for several hours. During this time the labelled hyaluronan will be transported back into the medium. The amount of labelled hyaluronan oligosaccharide in the medium can be determined by a biotin-related assay, by radioactivity or be fluorescence intensity.

### Example 9

### Identification of MRP5 as a Hyaluronan Transporter

### Materials

Valspodar was a kind gift from Novartis. Other chemicals were from Sigma Chemical Co.

### Cells and Cell Culture

Human fibroblasts were grown in suspension culture in Dulbecco's modified Eagles medium supplemented with streptomycin/penicillin (100 units of each/ml), kanamycin (100 units/ml) and 10% foetal calf serum. Cell proliferation was determined by cell counting after trypsinisation three days after seeding.

### Hyaluronan Synthase Activity

Cells from five culture flasks (180 cm2 growth area) were washed with cold phosphate buffered saline (PBS), harvested with the aid of a rubber policeman, sedimented at 1500 g for 5 min and suspended in 30 ml of ice-cold PBS. The cells were transferred into a Parr-cell disruption bomb, exposed to a nitrogen pressure of 900 psi for 15 min and disrupted by nitrogen cavitation [18] and the particulate fraction was obtained by centrifugation at 40000 g for 20 min. The sediment was suspended in 50 mM TRIS-malonate pH 7.0 at a protein concentration of 200 μg/ml and were mixed with an equal volume of the substrate for hyaluronan synthesis that contained 8 μM UDP-[14C]UDP-GlcA, 166 μM UDP-GlcNac, 4 mM dithiothreitol, 20 mM MgCl₂ in 50 mM TRIS-malonate pH 7.0 and incubated at 37° C. for 4 hours in the presence of increasing concentrations of multidrug resistance inhibitors. Hyaluronan synthesis was stopped by adding a solution of 10% sodium dodecylsuifate (SDS) to a final concentration of 1%. The mixtures were applied to descending paper chromatography that was developed with ethanol/aq. 1 M ammonium acetate pH 5.5 (13:7) as solvent. After 18 h the radioactivity of [14C]hyaluronan at the origin was determined.

### Hyaluronan Production

Trypsinised fibroblasts were suspended in Dulbecco's medium at 10⁵ cells/ml and 100 μl aliquots were transferred to a 96 well microtiter plate. The first row received 200 μl of the suspension and 20 μl of the multidrug resistance inhibitors dissolved in DMSO at concentrations of 4 mM. A serial dilution to the inhibitors was established by transfer of 100 μl aliquots from the first row to the following rows. All experiments were performed in duplicates. The last row did not receive any inhibitor and served as control. Prior to these experiments it was established that the DMSO concentration used did not influence hyaluronan production or cell proliferation. The cells were incubated for 2 days at 37° C. and aliquots (5 and 20 μl) of the culture medium were used for measurement of the hyaluronan concentration in the cell culture medium by an ELISA [19]. Briefly, the wells of a 96 well Covalink-NH-microtiter plate (NUNC) were coated with 100 μl of a mixture of 100 mg/ml of hyaluronan (Healon®), 9.2 μg/ml of N-Hydroxysuccinimide-3-sulfonic acid and 615 μl/ml of 1-ethyl-3-(3-dimethylaminopropyl)-carbodiimide for 2 hours at room temperature and overnight at 4° C. The wells were washed three times with 2 M NaCl, 41 mM MgSO₄, 0.05% Tween-20 in 50 mM phosphate buffered saline pH 7.2 (buffer A) and once with 2 M NaCl, 41 mM MgSO₄, in phosphate buffered saline pH 7.2. Additional binding sites were blocked by incubation with 300 μl of 0.5% bovine serum albumin in phosphate buffered saline for 30 min at 37° C. Calibration of the assay was performed with standard concentrations of hyaluronan ranging from 15 ng/ml to 6000 ng/ml in equal volumes of culture medium as used for measurement of the cellular supernatants. A solution (50 μl) of the biotinylated hyaluronan binding fragment of aggrecan (Applied Bioligands Corporation, Winnipeg, Canada) in 1.5 M NaCl, 0.3 M guanidinium hydrochloride, 0,08% bovine serum albumin 0.02% NaN3 25 mM phosphate buffer pH 7.0 was preincubated with 50 μl of the standard hyaluronan solutions or cellular supernatants for 1 hour at 37° C. The mixtures were transferred to the hyaluronan-coated test plate and incubated for 1 hour at 37° C. The microtiter plate was washed three times with buffer A and incubated with 100 μl/well of a solution of streptavidin-horseraddish-peroxidase (Amersham) at a dilution of 1:100 in phosphate buffered saline, 0.1% Tween-20 for 30 min at room temperature. The plate was washed five times with buffer A and the colour was developed by incubation with a 100 μl/well of a solution of 5 mg o-phenylenediamine and 5 μl 30% H2O2 in 10 ml of 0.1 M citrate-phosphate buffer pH 5.3 for 25 min at room temperature. The adsorption was read at 490 nm. The concentrations in the samples were calculated from a logarithmic regression curve of the hyaluronan standard solutions.

### Results

### Homology Search for Eukaryotic Hyaluronan Transporters

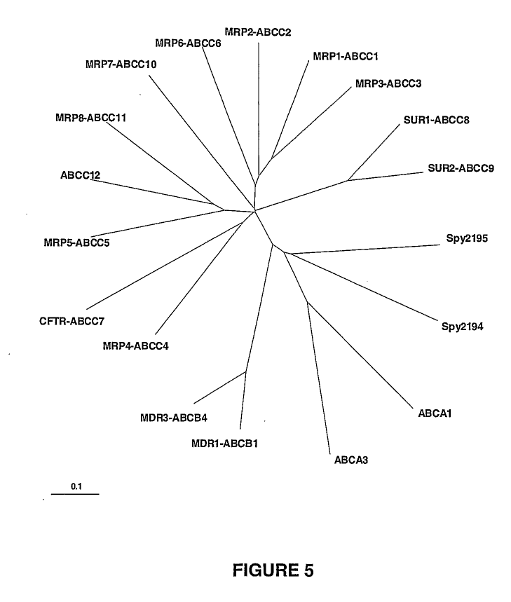

The protein sequences of the streptococcal hyaluronan ABC-transporter Spy2194 and Spy2195 were used to search for homologous human sequences. The highest homology was found with ABCA, ABCB and ABCC transporters, three protein families of the large group of ABC-transporters. The phylogenetic relationship is shown in FIG. 5.

### Effect on ABC-Transport Inhibitors on Hyaluronan Synthesis and Proliferation of Human Skin Fibroblasts

The structural homology of the streptococcal hyaluronan transporter with human multidrug resistant transporter could correspond well with identical functions. Because a wide range of multidrug transport inhibitors was available, a selected set of compounds were applied to human skin fibroblasts. The inhibitors were selected according to their discriminatory capacity to distinguish the different members multidrug resistance transporter. Three parameters were measured: 1. The amount of hyaluronan produced during growth into the culture supernatant. 2. The hyaluronan synthase activity in particulate membrane fractions to eliminate the possibility that inhibition of hyaluronan production in cell culture could be caused indirectly by cellular mediators. 3. The effect on cell proliferation, because hyaluronan synthesis is also required for detachment during mitosis and growth of the human HT1080 cell line [14].

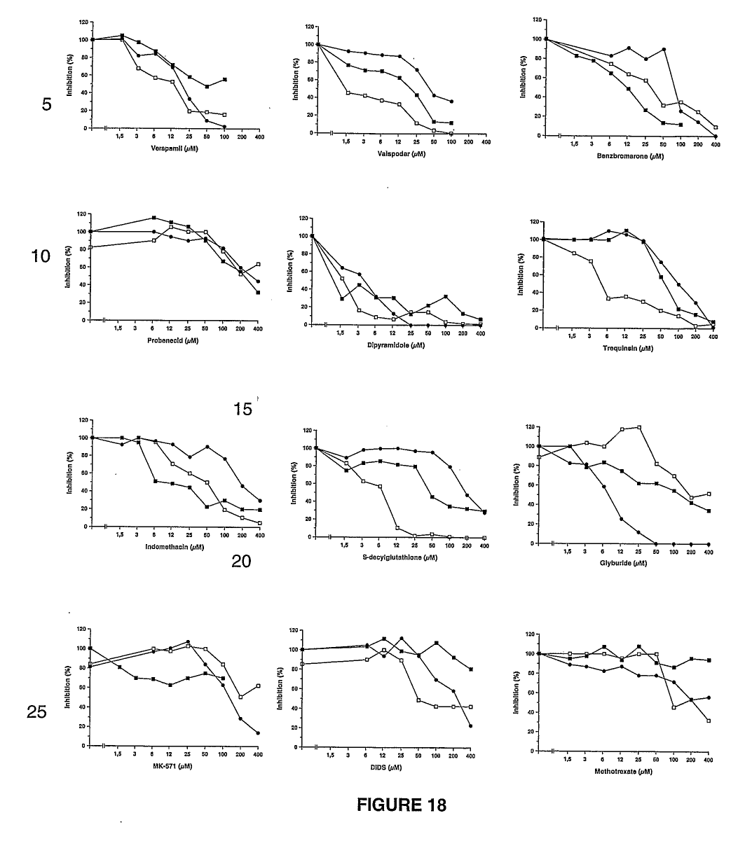

The concentration dependency for 12 different inhibitors is shown in FIG. 18 A-L and the IC50-concentrations deduced from these results are summarized in Table 1. Several conclusions can be drawn from these results. Some inhibitors decreased not only hyaluronan production but also the synthase activity at concentrations that were in the same range usual for the transport inhibition of other known multidrug resistance transporter substrates. This finding indicated that a transporter function was required for hyaluronan export from human fibroblasts and this transporter was a member of the multidrug resistance transporter family. The direct inhibition of the synthase activity eliminated any interference from intracellular metabolites and this observation indicated that the hyaluronan synthase activity and export were coordinated.

These inhibitors have a specific profile for certain members of the ABC transporter family and the published inhibitory concentrations (IC50 or Ki) are included in Table 1. A comparison of known inhibitory concentrations with that for the hyaluronan secretory activity could indicate which transporter was most likely for hyaluronan export. Thus, the inhibitors can be broadly arranged into two groups (Table 1): group 1 contains those inhibitors that decreased hyaluronan synthase activity at concentrations comparable with other secreted drugs and group 2 contains those that inhibited hyaluronan synthase activity at higher concentrations or not at all. Some inhibitors of group 1A such as verapamil and Valspodar block a broad spectrum of transporter proteins and inhibited also hyaluronan synthesis indicating that the transporter belonged to the MDR- or MRP-family, which are both blocked by these.

The inhibitors of group 1B, indomethacin and the organic anion transport inhibitors benzbromarone and probenecid, in contrast preferentially inhibit MRP- and not MDR-transporters. As the latter three inhibited hyaluronan transport, a transport across the membrane by a member of the MDR-family appears unlikely, as the members of the MDR family are not blocked by these inhibitors. The inhibitors of group 1C block MRP transporters differentially and can discriminate them. Dipyramidole is an effective inhibitor of MRP1, MRP4 and MRP5 and also of hyaluronan transport. Therefore one of these three transporters is a likely hyaluronan transporter. Trequinsin, a potent inhibitor of MRP4 and MRP5 transport, was also a good inhibitor of hyaluronan transport indicating that one or both of these transporters are likely hyaluronan transporters as well. S-decylglutathione, an inhibitor of all MRP-, but the MRP3-transporter, was a very good inhibitor of hyaluronan transport, hence the MRP3 transporter can be excluded as a hyaluronan transporter. Glyburide, an efficient inhibitor of ABC-A and MRP4 transporters, only inhibits at concentrations aboye 50 μM and the inhibition was not more than 50% even at 400 μM, thus ABCA1 and MRP4 transporter proteins can be excluded as likely candidates for transporting hyaluronan. MK-571 and methotrexate are good inhibitors for MRP1-4 and MRP7, but not for MRP5, both did not inhibit hyaluronan synthesis indicating again that the MRP1-4 and MRP7 transporters were unlikely candidates. DIDS preferentially inhibits ABC-A and the transport of inorganic anions and has low specificity for MRP transporters. It inhibited hyaluronan transport partially at relatively high concentrations, again excluding the ABC-A transporters and making the MRP transporters more likely candidates. All these data are compatible with hyaluronan transport preferentially by MRP5.

The export of hyaluronan from the synthase through the plasma membrane into the extracellular matrix by multidrug-resistance transporter explains many so far unresolved enigmas of hyaluronan synthesis. The presence of intracellular hyaluronan under certain growth conditions has so far been explained by cellular uptake [20,21]. However, this hypothesis could not satisfactorily explain how intact hyaluronan could bypass the breakdown process in lysosomes. Similarly, intracellular hyaluronan binding proteins have been found, however, and no an appropriate intracellular function had been proposed for them [22,23]. By demonstrating that membrane transporters block hyaluronan export through the plasma membrane, hyaluronan synthesis has now been assigned to take place in the cytoplasm, which would resolve both above mentioned problems.

Comparing the known IC50 or Ki-concentrations for a set of multidrug-resistance inhibitors with the inhibitory concentrations of hyaluronan synthesis suggested that MRP5 is the principle hyaluronan transporter in human fibroblasts.

The drugs verapamil and Valspodar have rather broad transporter blocking specificities, as they inhibit MDR- as well as MRP-transporters. Verapamil is a licensed calcium channel blocker clinically used to treat arhythmias, in addition to this function it also inhibits MDR1 with a Ki=120 μM [24]. The IC50 concentrations for MDR1 and MRP have been reported be 2-5 μM and 4-8 μM, respectively [8]. It also inhibited the MRP4 mediated export of methotrexate with an IC50-30 μM [25]. It inhibits the export of fluorescein diacetate at a concentration of 25 μM by MRP5 [26].

Valspodar (PSC833) is a cyclosporin derivative with low toxicity and inhibited the MDR1 mediated transport of rhodamine with a Ki=0.75 μM [27] and transport of leukotriene C4 by MRP1 with a Ki=27 μM [28] and by MRP2 with a Ki=28.9 μM [29]. It also inhibited the MRP4 mediated export of methotrexate with an IC50˜10 μM [25]. Verapamil and Valspodar inhibited with IC50 of about 50 μM and 15 μM, respectively. Thus members of the MDR and MRP family are required for hyaluronan export.

Benzbromarone and Probenecid are general inhibitors for organic anions that block reasorption of uric acid in the epithelia of kidney tubules. Benzbromarone is a general inhibitor of organic anion transporters that inhibits MRP1 with IC50=4 μM, both MRP4 and MRP5 with IC50=150 μM and MDR1 with IC50>800 μM [8]. Another study reported an IC50<5 μM for the MRP5 mediated transport of S-(2,4-dinitrophenyl)glutathione [30]. The 9-(2-phosphonomethoxyethyl)adenine (PMEA) transport by MRP4 or MRP5 was inhibited with IC50=150 μM [31]. It has no specificity for MDR transporters and was a very effective inhibitor of hyaluronan transport with an IC50-25 μM. This excluded the MDR transporter as hyaluronan transporter.

Probenecid preferentially inhibits MRP transporters at rather high concentrations of IC50=500-800 μM, whereas it has almost no inhibitory capacity towards MDR1 (IC50>2000 μM) [8]. Other reports measured its action on the MRP transporters of human erythrocytes that do not express substantial amounts of MDR1 and found an IC₅₀=100-200 μM [32,33]. It also inhibited the MRP4 mediated export of methotrexate with an IC₅₀˜300 μM [25]. It inhibited the MRP5 mediated transport of cGMP at a concentration of 50 μM by 68% [34]. The 9-(2-phosphonomethoxyethyl)adenine (PMEA, an anti-HIV-drug) transport by MRP4 was very resistant to inhibition with an IC50=2300 μM, but sensitive by MRP5 with an IC50=200 μM [31]. It inhibits the export of the fluorescent dye fluorescein diacetate at a concentration of 1 mM by MRP5 [26]. It was a weak inhibitor of the 17β-glucuronosyl-estradiol export by MRP7 [35]. Thus probenecid is a poor inhibitor for MDR1 and MRP4 and a rather good inhibitor for MRP5. Its inhibitory concentration for hyaluronan transport falls into a similar range as for MRP5.

Dipyramidol and Trequinsin are phosphodiesterase inhibitors. Dipyramidole is a vasodilator that is used to decrease the resistance coronary arteries. It inhibited the 9-(2-phosphonomethoxyethyl)adenine (PMEA) transport by MRP4 with an IC50=2 μM, and by MRP5 with an IC50=30 μM [31]. Another study reported an IC50>10 μM for the MRP5 mediated transport of S-(2,4-dinitrophenyl)glutathione [30]. Dipyramidole is an effective inhibitor of MRP1, MRP4 and MRP5 and of hyaluronan transport. Therefore one of these transporters is a likely hyaluronan transporter.

Trequinsin, a potent phosphodiesterase inhibitor, inhibited the MRP4 mediated export of methotrexate with an IC50˜10 μM [25]. It inhibits the MRP5 mediated transport of cGMP with a Ki=0.24 μM and of cAMP with a Ki=0.38 μM [34], but inhibits the export of 17β-glucuronosyl-estradiol by MRP7 only moderately at 100 μM by 44% [35]. The 9-(2-phosphonomethoxyethyl)adenine (PMEA) transport by MRP4 was inhibited with an IC50=10 μM, and by MRP5 with an IC50=30 μM [31]. Trequinsin, a potent inhibitor of MRP4 and MRP5 transport, was also a good inhibitor of hyaluronan transport. These experiments limit the likely hyaluronan transporters to MRP 4 and 5.

Indomethacin is a weak acid and is used in antirheumatic therapy as the first choice of nonsteriodal antirheumatic drugs (NSARD), because it inhibits the cyclooxygenase that is required for prostaglandin E2 synthesis, a mediator of pain and inflammation. It has already been shown that the indomethacin and its relative mefenamic acid inhibit hyaluronan synthesis in fibroblasts [36]. Indomethacin has a similar inhibitory spectrum as benzbromarone with IC50>800 μM for MDR1 and IC50=10-20 μM for the MRP transporter [8,32]. It inhibits the MRP4 mediated transport of cGMP with an IC50-20˜50 μM [33] and the transport of 17β-glucuronosyl-estradiol by MRP4 with an IC50˜5 μM and by MRP1 with an IC50˜50 μM [37]. Another study reported an IC50>100 μM for the MRP5 mediated transport of S-(2,4-dinitrophenyl)glutathione [30]. Indomethacin is an inhibitor of all members of the MRP transporter family, but it does not inhibit any member of the MDR transporter family, and as it acts on the former transporter family showed a very good inhibition of hyaluronan synthesis. This finding again excluded the MDR transporter family as a hyaluronan transporter.

S-decylglutathione inhibited the leukotriene export by MRP1 with IC50=37 μM [38]. Simultaneously it stimulated the ATPase activity of the nucleotide binding domains of MRP1 (allocrites) [39]. S-decylglutathione, an inhibitor of transport of glutathione conjugates by MRP1, MRP2, MRP4 and MRP5 with low affinity for MRP3, was a very good inhibitor of hyaluronan transport, hence excluding the MRP3 transporter for hyaluronan.

Glyburide (glibenclamide) is a typical inhibitor of ABCA1 transporter that inhibits the secretion of macrophage inhibitory factor with IC50 concentrations of about 5 μM [40]. However, it also inhibited the export calcein (a fluorescent anionic dye substrate) by MRP1 in the concentration range of 10 μM to 50 μM [41] and the MDR1 mediated export of colchicine at a concentration of about 100 μM, because it is a substrate for MDR1 itself [42]. It is a very effective inhibitor of the MRP4 mediated transport of cGMP in human erythrocytes with an IC50=1.9 μM [33]. Glyburide, an efficient inhibitor of ABC-A and MRP4, only inhibits hyaluronan secretion at concentrations above 50 μM and the inhibition did not exceed 50% even at 400 μM. From these data we excluded the ABCA1 and MRP4 transporter as likely candidates.

MK-571, a leukotriene analogue, inhibits preferentially MRP1 with Ki=0.6 □M [28] and has an IC50=0.11 μM for leukotriene export [38]. Its inhibitory activity is much lower towards MRP2 with an IC50=469 μM [43]. Another study reported a Ki=13.1 μM for the MRP2 mediated export of leukotriene C4 [29]. It inhibited the 17β-glucuronosyl-estradiol export by MRP7 at 30 μM by 42% [35]. It also inhibited the MRP4 mediated export of methotrexate with an IC50˜1 μM [25]. It has no inhibitory effect on the MRP5 mediated transport of cGMP at concentrations up to 50 μM. The 9-(2-phosphonomethoxyethyl)adenine (PMEA) transport by MRP4 was sensitive to inhibition with an IC50=10 μM, but relatively resistant by MRP5 with an IC50=40 μM [31]. MK-571 is a good inhibitor for MRP1-4 and MRP7, but not for MRP5. This drug inhibited hyaluronan production only partially at concentrations above 100 μM. If an inhibitor selected from MRP1-4 and MRP7 were responsible for hyaluronan transport, a more efficient inhibition would be expected. But this was not the case. Therefore the MRP1-4 and MRP7 transporters do not appear to be preferred hyaluronan transporters in human fibroblasts.

DIDS (4,4-diisothiocyanato-stilbene-2,2-disulphonate) is also an inhibitor of anion transport, but it prefers inorganic anion transporters such as the chloride channel with a Ki=0.84 μM [44], however it has also low inhibitor action on the MRP transporters of human erythrocytes (IC50=150-300 μM) [32]. DIDS preferentially inhibits ABC-A and inorganic anions and has low specificity for MRP transporters. It inhibited hyaluronan transport partially at relatively high concentrations, again excluding the ABC-A transporters and making MRP transporters more likely candidates for hyaluronan transport across the cell membrane.

Methotrexate is a substrate for MRP1, MRP2, MRP3 and MRP4 [45], but not for MRP5 [46]. It did not inhibit the interleukin 1 stimulated secretory activities of cultured human synovial fibroblasts [47]. It also did not inhibit the hyaluronan synthase activity making MRP1, MRP2, MRP3 and MRP4 unlikely as hyaluronan exporters.

MRP5 analysis is still in its infancy. It is an organic anion transporter and most closely related to MRP4. MRP5 transports the fluorescent dye fluorescein diacetate [34], cAMP and cGMP and of glutathione conjugated compounds such as S-(2,4-dinitrophenyl)glutathione [30], but not leukotriene C4, 17β-glucuronosyl-estradiol, calcein, GSSG, and PGE1 or PGE2 [37]. It can be expressed in several splicing variants [48]. Knock out mice do not display obvious abnormalities, despite the fact that MRP5 is expressed in all tissues analysed thus far [26,49].

From the inhibitory profile of the drugs it can be concluded that MRP5 the most likely hyaluronan transporter of human fibroblasts. This conclusion does not imply that MRP5 transports hyaluronan exclusively, as MRP5 knock out mice are viable thus indicating that alternative transport systems within cells can compensate for the lack of MRP5. These transporters could be ABCC11 or ABCC12 due to their close phylogenetic relationship [50]. Hyaluronan deficiency would be expected to be incompatible with life, because it is required for cell differentiation immediately after fertilization [51] as well as for fibroblasts proliferation [1,4], both observations highlighted by the fact that hyaluronan deficient knockout mice die at the stage 10.5 [52]. Hence other channels must mediate hyaluronan secretion in MRP5 knockout mice. In their publication Reid et al. ended their discussion on the function of MRP4 and MRP5 with the statement: “For MRP5, a high-affinity substrate remains to be found” [31], which we herewith present.

As most tissues contain hyaluronan, the ubiquitous tissue distribution of MRP5 also supports the hypothesis that it is a hyaluronan transporter, while most of the other MRP transporters such as MRP2, MRP3 and MRP4 have a much more restricted tissue distribution making them unlikely candidates for hyaluronan transport. The broad expression of MRP5 also extends to cartilage, because MRP5 mRNA could be detected in human chondrosarcoma cells (own unpublished observation). It is interesting that MRP5 is present in the brain [49] and even in various regions of the brain [26] matching the observation that hyaluronan is synthesized in the various regions of the brain as well [53]. The only other MRP family member known to reside in the brain is MRP1, which is restricted to the choroid plexus, making this member of the MRP family an unlikely transporter of hyaluronan. MRP5 is also expressed in cardiac muscle cells of human heart and is enhanced under ischemic conditions [54], which correlates well with the observation that hyaluronan synthesis is increased in myocardial infarction [55]. Thus, the tissue distribution of MRP5 and hyaluronan are in broad agreement with the proposed function of MRP5 as a hyaluronan transporter.

Our conclusion that a multidrug resistant transporter is involved in hyaluronan export is further supported by the observation that increased hyaluronan production induced resistance in drug-sensitive tumor cells [56]. The mechanism of this inhibition could simply be explained by competition of two substrates for the transporter.

Our observation that both hyaluronan synthase and transport were inhibited simultaneously implies that these activities were coordinated within the plasma membranes in such a way that growing hyaluronan chains exerted a feedback inhibition on the synthase. This phenomenon has been described previously [57,58]. The results also showed that inhibition of hyaluronan synthesis reduced cell proliferation and verified previous studies [14]. A similar coordination of synthesis and export has previously been observed for polysialic acid [59].

Finally, the our discovery of drugs that are used for other diseases for inhibition of hyaluronan synthesis may open novel ways for treatment of diseases that are characterized by HA overproduction such as oedema formation after injuries, inflammation and metastasis.

TABLE 1
Inhibitory concentrations (IC₅₀) of selected drugs for proliferation, hyaluronan synthase activity,
and hyaluronan production of human fibroblasts and comparison with published data for ABC transporter.

| | | | | HA | | | | | | | | |
|---|---|---|---|---|---|---|---|---|---|---|---|---|
| | | Prolife- | HA | synthase | | | | | | | | |
| | | ration | production | activity | published inhibitory concentrations IC₅₀ or Kᵢ (μM) | | | | | | | |
| Group | Inhibitor | (μM) | (μM) | (μM) | ABCA | MDR1 | MRP1 | MRP2 | MRP3 | MRP4 | MRP5 | MRP7 |
| 1A | Verapamil | 20 | 15 | ~50 | | 2-5 | | | | 30 | 25 | |
| 1A | Valspodar | 45 | 15 | 20 | | 0.75 | 27 | 28.9 | | 10 | | |
| 1B | Benzbromarone | 75 | 30 | 12 | | >800 | 4 | | | 150 | 150; <5 | |
| 1B | Probenecid | 200 | 200 | 200 | | >2000 | 100-200 | | | 300 | 200 | weak |
| 1B | Indomethacin | 200 | 50 | 10 | | >800 | 10-20; 50 | | | 20-50; 5 | >100 | |
| 1C | Dipyramidole | 4 | 15 | ~1 | | | | | | 2 | 10; 30 | |
| 1C | Trequinsin | 100 | 6 | 60 | | | | | | 10 | 0.24; 30 | ~100 |
| 1C | S- | 190 | 7 | 40 | | | 37 | good | low | good | good | |
| | decylglutathione | | | | | | | inhibitor | affinity | inhibitor | inhibitor | |
| 2 | Glyburide | 7 | ~400 | 120 | 5 | 100 | 10-50 | | | 1.9 | | |
| 2 | MK-571 | 150 | >400 | >100 | | 0.11; | 469; | | | 1 | no | ~30 |
| | | | | | | 0.6 | 13.1 | | | | inhibition | |
| 2 | DIDS | 250 | 50 | >400 | 0.84 | | 150-300 | | | 150-300 | | |
| 2 | methotrexate | ~200 | ~200 | >400 | | | substrate | substrate | substrate | substrate | no | |
| | | | | | | | | | | | substrate | |

### REFERENCES

### For Example 9

[1] Prehm P. Hyaluronan. In: Biopolymers (Ed. Steinbüchel A) 2002:15:379-406. Whiley-VCH-Verlag, Weinheim, Germany.
[2] Prehm P. Synthesis of hyaluronate in differentiated teratocarcinoma cells. Mechanism of chain growth. Biochem J 1983; 211:191-8.
[3] Prehm P. Synthesis of hyaluronate in differentiated teratocarcinoma cells. Characterization of the synthase. Biochem J 1983; 211:181-9.
[4] Prehm P. Hyaluronate is synthesized at plasma membranes. Biochem J 1984; 220:597-600.
[5] Tlapak-Simmons V L, Kempner E S, Baggenstoss B A, and Weigel P H. The active streptococcal hyaluronan synthases (HASs) contain a single HAS monomer and multiple cardiolipin molecules. J Biol Chem 1998;273:26100-9.
[6] Pummill P E, Kempner E S, and DeAngelis P L. Functional molecular mass of a vertebrate hyaluronan synthase as determined by radiation inactivation analysis. J Biol Chem 2001;276:39832-5.
[7] Schinkel A H and Jonker J W. Mammalian drug efflux transporters of the ATP binding cassette (ABC) family: an overview. Adv Drug Deliv Rev 2003; 55:3-29.
[8] Hollo Z, Homolya L, Hegedus T, and Sarkadi B. Transport properties of the multidrug resistance-associated protein (MRP) in human tumour cells. FEBS Lett 1996; 383:99-104.
[9] Becq F, Hamon Y, Bajetto A, Gola M, Verrier B, and Chimini G. ABC1, an ATP binding cassette transporter required for phagocytosis of apoptotic cells, generates a regulated anion flux after expression in Xenopus laevis oocytes. Biol Chem 1997; 272:2695-9.
[10] von Eckardstein A, Langer C, Engel T, Schaukal I, Cignarella A, Reinhardt J, Lorkowski S, Li Z, Zhou X, Cullen P, and Assmann G. ATP binding cassette transporter ABCA1 modulates the secretion of apolipoprotein E from human monocyte-derived macrophages. FASEB J 2001; 15:1555-61.
[11] Wiese M and Pajeva IK. Structure-activity relationships of multidrug resistance reversers. Curr Med Chem 2001; 8:685-713.
[12] Borst P, Evers R, Kool M, and Wijnholds J. A family of drug transporters: the multidrug resistance-associated proteins. J Natl Cancer Inst 2000; 92:1295-302.
[13] Kruh G D and Belinsky M G. The MRP family of drug efflux pumps. Oncogene 2003; 22:7537-52.
[14] Brecht M, Mayer U, Schlosser E, and Prehm P. Increased hyaluronate synthesis is required for fibroblast detachment and mitosis. Biochem J 1986; 239:445-50.
[15] Ueki N, Taguchi T, Takahashi M, Adachi M, Ohkawa T, Amuro Y, Hada T, and Higashino K. Inhibition of hyaluronan synthesis by vesnarinone in cultured human myofibroblasts. Biochim Biophys Acta 2000; 1495:160-7.
[16] Nakamura T, Funahashi M, Takagaki K, Munakata H, Tanaka K, Saito Y, and Endo M. Effect of 4-methylumbelliferone on cell-free synthesis of hyaluronic acid. Biochem Mol Biol Int 1997; 43:263-8.
[17] Nakamura T, Takagaki K, Shibata S, Tanaka K, Higuchi T, and Endo M. Hyaluronic-acid-deficient extracellular matrix induced by addition of 4-methylumbelliferone to the medium of cultured human skin fibroblasts. Biochem Biophys Res Commun 1995; 208:470-5.
[18] Klempner M S, Mikkelsen R B, Corfman D H, and Andre S J. Neutrophil plasma membranes. I. High-yield purification of human neutrophil plasma membrane vesicles by nitrogen cavitation and differential centrifugation. J Cell Biol 1980; 86:21-8.
[19] Stern M and Stern R. An ELISA-like assay for hyaluronidase and hyaluronidaseinhibitors. Matrix 1992; 12:397-403.
[20] Evanko S P and Wight T N. Intracellular localization of hyaluronan in proliferating cells. J Histochem Cytochem 1999; 47:1331-41.
[21] Tammi R, Rilla K, Pienimaki J P, MacCallum D K, Hogg M, Luukkonen M, Hascall V C, and Tammi M. Hyaluronan enters keratinocytes by a novel endocytic route for catabolism. J Biol Chem 2001;276:35111-22.
[22] Assmann V, Jenkinson D, Marshall J F, and Hart I R. The intracellular hyaluronan receptor RHAMM/IHABP interacts with microtubules and actin filaments [In Process Citation]. J Cell Sci 1999; 112 (Pt 22):3943-54.
[23] Huang L, Grammatikakis N, Yoneda M, Banerjee S D, and Toole B P. Molecular characterization of a novel intracellular hyaluronan-binding protein. Biol Chem 2000; 275:29829-39.
[24] Buxbaum E. Co-operative binding sites for transported substrates in the multiple drug resistance transporter Mdrl. Eur J Biochem 1999; 265:64-70.
[25] Chen Z S, Lee K, Walther S, Raftogianis R B, Kuwano M, Zeng H, and Kruh G D. Analysis of methotrexate and folate transport by multidrug resistance protein 4 (ABCC4): MRP4 is a component of the methotrexate efflux system. Cancer Res 2002; 62:3144-50.
[26] McAleer M A, Breen M A, White N L, and Matthews N. pABC11 (also known as MOAT-C and MRP5), a member of the ABC family of proteins, has anion transporter activity but does not confer multidrug resistance when overexpressed in human embryonic kidney 293 cells. J Biol Chem 1999; 274:23541-8.
[27] Robey R, Bakke S, Stein W, Meadows B, Litman T, Patil S, Smith T, Fojo T, and Bates S. Efflux of rhodamine from CD56+ cells as a surrogate marker for reversal of P-glycoprotein-mediated drug efflux by PSC 833. Blood 1999; 93:306-14.
[28] Leier I, Jedlitschky G, Buchholz U, Cole S P, Deeley R G, and Keppler D. The MRP gene encodes an ATP-dependent export pump for leukotriene C4 and structurally related conjugates. J Biol Chem 1994; 269:27807-10.
[29] Chen Z S, Kawabe T, Ono M, Aoki S, Sumizawa T, Furukawa T, Uchiumi T, Wada M, Kuwano M, and Akiyama Si. Effect of multidrug resistance-reversing agents on transporting activity of human canalicular multispecific organic anion transporter. Mol Pharmacol 1999; 56:1219-28.
[30] Wijnholds J, Mol C A, van Deemter L, De Haas M, Scheffer G L, Baas F, Beijnen J H, Scheper R J, Hatse S, De Clercq E, Balzarini J, and Borst P. Multidrug-resistance protein 5 is a multispecific organic anion transporter able to transport nucleotide analogs. Proc Natl Acad Sci USA 2000; 97:7476-81.
[31] Reid G, Wieling a P, Zelcer N, De Haas M, van Deemter L, Wijnholds J, Balzarini J, and Borst P. Characterization of the transport of nucleoside analog drugs by the human multidrug resistance proteins MRP4 and MRP5. Mol Pharmacol 2003; 63:1094-103.
[32] Bobrowska-Hagerstrand M, Wrobel A, Rychlik B, Bartosz G, Soderstrom T, Shirataki Y, Motohashi N, Molnar J, Michalak K, and Hagerstrand H. Monitoring of MRP-like activity in human erythrocytes: inhibitory effect of isoflavones. Blood Cells Mol Dis 2001; 27:894-900.
[33] Klokouzas A, Wu C P, van Veen H W, Barrand M A, and Hladky S B. cGMP and glutathione-conjugate transport in human erythrocytes. Eur J Biochem 2003; 270:3696-708.
[34] Jedlitschky G, Burchell B, and Keppler D. The multidrug resistance protein 5 functions as an ATP-dependent export pump for cyclic nucleotides. J Biol Chem 2000; 275:30069-74.
[35] Chen Z S, Hopper-Borge E, Belinsky M G, Shchaveleva I, Kotova E, and Kruh G D. Characterization of the transport properties of human multidrug resistance protein 7 (MRP7, ABCC10). Molecular Pharmacology 2003; 63:351-8.
[36] August E M, Nguyen T, Malinowski N M, and Cysyk R L. Non-steroidal anti-inflammatory drugs and tumor progression: Inhibition of fibroblast hyaluronic acid production by indomethacin and mefenamic acid. Cancer Lett 1994; 82:49-54.
[37] Reid G, Wieling a P, Zelcer N, Van D H, I, Kuil A, De Haas M, Wijnholds J, and Borst P. The human multidrug resistance protein MRP4 functions as a prostaglandin efflux transporter and is inhibited by nonsteroidal antiinflammatory drugs. Proc Natl Acad Sci USA 2003; 100:9244-9.
[38] Sundkvist E, Jaeger R, and Sager G. Leukotriene C(4) (LTC(4)) does not share a cellular efflux mechanism with cGMP: characterisation of cGMP transport by uptake to inside-out vesicles from human erythrocytes. Biochim Biophys Acta 2000; 1463:121-30.
[39] Cool R H, Veenstra M K, van Klompenburg W, Heyne R I, Muller M, de Vries E G, van Veen H W, and Konings W N. S-decyl-glutathione nonspecifically stimulates the ATPase activity of the nucleotide-binding domains of the human multidrug resistance-associated protein, MRP1 (ABCC1). Eur J Biochem 2002; 269:3470-8.
[40] Flieger O, Engling A, Bucala R. Lue H, Nickel W, and Bernhagen J. Regulated secretion of macrophage migration inhibitory factor is mediated by a non-classical pathway involving an ABC transporter. FEBS Lett 2003; 551:78-86.
[41] Payen L, Delugin L, Courtois A, Trinquart Y, Guillouzo A, and Fardel O. The sulphonylurea glibenclamide inhibits multidrug resistance protein (MRP1) activity in human lung cancer cells. Br J Pharmacol 2001; 132:778-84.
[42] Golstein P E, Boom A, van Geffel J, Jacobs P, Masereel B, and Beauwens R. P-glycoprotein inhibition by glibenclamide and related compounds. Pflugers Arch 1999; 437:652-60.
[43] Luo F R, Paranjpe P V, Guo A, Rubin E, and Sinko P. Intestinal transport of irinotecan in Caco-2 cells and MDCK II cells overexpressing efflux transporters Pgp, cMOAT, and MRP1. Drug Metab Dispos 2002; 30:763-70.
[44] Dick G M, Kong I D, and Sanders K M. Effects of anion channel antagonists in canine colonic myocytes: comparative pharmacology of Cl−, Ca2+ and K+ currents. Br J Pharmacol 1999; 127:1819-31.
[45] Assaraf Y G, Rothem L, Hooijberg J H, Stark M, Ifergan I, Kathmann I, Dijkmans B A, Peters G J, and Jansen G. Loss of multidrug resistance protein 1 expression and folate efflux activity results in a highly concentrative folate transport in human leukemia cells. J Biol Chem 2003; 278:6680-6.
[46] Stark M, Rothem L, Jansen G, Scheffer G L, Goldman I D, and Assaraf Y G. Antifolate resistance associated with loss of MRP1 expression and function in Chinese hamster ovary cells with markedly impaired export of folate and cholate. Mol Pharmacol 2003; 64:220-7.
[47] Meyer F A, Yaron I, Mashiah V, and Yaron M. Methotrexate inhibits proliferation but not interleukin I stimulated secretory activities of cultured human synovial fibroblasts. J Rheumatol 1993; 20:238-42.
[48] Suzuki T, Sasaki H, Kuh H J, Agui M, Tatsumi Y, Tanabe S, Terada M, Saijo N, and Nishio K. Detailed structural analysis on both human MRP5 and mouse mrp5 transcripts. Gene 2000; 242:167-73.
[49] Kool M, De Haas M, Scheffer G L, Scheper R J, van Eijk M J, Juijn J A, Baas F, and Borst P. Analysis of expression of CMOAT (MRP2), MRP3, MRP4, and MRP5, homologues of the multidrug resistance-associated protein gene (MRP1), in human cancer cell lines. Cancer Res 1997; 57:3537-47.
[50] Tammur J, Prades C, Arnould I, Rzhetsky A, Hutchinson A, Adachi M, Schuetz J D, Swoboda K J, Ptacek L J, Rosier M, Dean M, and Allikmets R. Two new genes from the human ATP-binding cassette transporter superfamily, ABCC11 and ABCC12, tandemly duplicated on chromosome 16q12. Gene 2001; 273:89-96.
[51] Prehm P. Induction of hyaluronic acid synthesis in teratocarcinoma stem cells by retinoic acid. FEBS Lett 1980; 111:295-8.
[52] Fulop C, Salustri A, and Hascall V C. Coding sequence of a hyaluronan synthase homologue expressed during expansion of the mouse cumulus-oocyte complex. Archives of Biochemistry and Biophysics 1997; 337:261-6.
[53] Bignami A and Asher R. Some observations on the localization of hyaluronic acid inadult, newborn and embryonal rat brain. Int J Dev Neurosci 1992; 10:45-57.
[54] Dazert P, Meissner K, Vogelgesang S, Heydrich B, Eckel L, Bohm M, Warzok R, Kerb R, Brinkmann U, Schaeffeler E, Schwab M, Cascorbi I, Jedlitschky G, and Kroemer H K. Expression and localization of the multidrug resistance protein 5 (MRP5/ABCC5), a cellular export pump for cyclic nucleotides, in human heart. Am J Pathol 2003; 163:1567-77.
[55] Waldenstrom A, Martinussen H J, Gerdin B, and Hatllgren R. Accumulation of hyaluronan and tissue edema in experimental myocardial infarction. J Clin Invest 1991; 88:1622-8.
[56] Misra S, Ghatak S, Zoltan-Jones A, and Toole B P. Regulation of multi-drug resistance in cancer cells by hyaluronan. J Biol Chem 2003; 278:25285-8.
[57] Nickel V, Prehm S, Lansing M, Mausolf A, Podbielski A, Deutscher J, and Prehm P. An ectoprotein kinase of group C streptococci binds hyaluronan and regulates capsule formation. J Biol Chem 1998; 273:23668-73.
[58] Luke H J and Prehm P. Synthesis and shedding of hyaluronan from plasma membranes of human fibroblasts and metastatic and non-metastatic melanoma cells. Biochem J 1999; 343:71-5.
[59] Bliss J M and Silver R P. Coating the surface: a model for expression of capsular polysialic acid in Escherichia coli K1. Mol Microbiol 1996; 21:221-31.

### Example 10

### Inhibition of MRP5 by RNAi

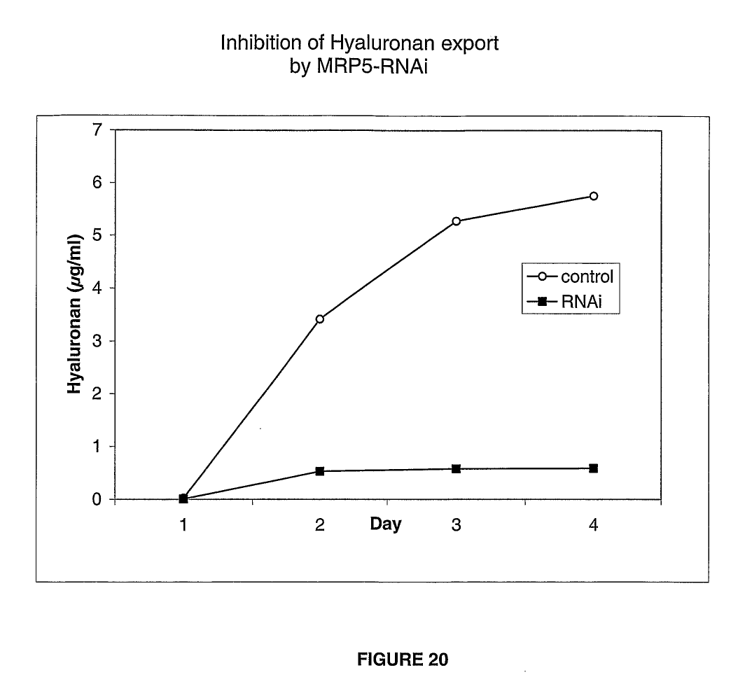

We conducted a specific inhibition of the MRP5 transporter in human fibroblasts by the method of RNA interference (RNAi). A human fibroblast culture was transfected with MRP5-RNAi and after various time periods the amount of hyaluronan in the culture medium was determined by an ELISA method as described in the previous examples. FIG. 20 shows the amount of normal human fibroblast culture as compared to the MRP5—RNAi transfected culture. It is evident that MRP5-RNAi strongly inhibited hyaluronan production.

### Method

The pcDNA3.1/HygroMRP5 Clone containing the human MRP5 gene was obtained from Dr. Jedlitschky [1]. The MRP5 gene was amplified by PCR using the primer pairs listed in Table 1.

TABLE 1

Sequence

| | |
|---|---|
| 5-MRP5-RNAi | 5-ATG ACT TTT TCG TGG CTT TCT TCT-3 |
| 3-MRP5-RNAi | 5-GCT TTG ACC CAG GCA TAC ATT TT-3 |
| 5-MRP5- | 5-GAC TTG GGC ATT GAA TTA CCG AAC-3 |
| RNAi/713 | |
| 3-MRP5- | 5-GTG AGG ACT GGC TGG TTT GTT CTT-3 |
| RNAi/1460 | |
| 5-MRP5- | 5-GGG AAA GTA CCA TCA TGG CTT GAG-3 |
| RNAi/233 | |
| 3-MRP5- | 5-GAA AAT GCT TTG ACC CAG GCA TAC-3 |
| RNAi/1132 | |
| 5-humABCC5 | 5-GCC CTG CTG CGC CAC TGT AAG ATT-3 |
| 3-humABCC5 | 5-TCC GGA ACT GCT GTG CGA AAG ATA AA-3 |

Double stranded RNAi was obtained using the Block-iT Complete Dicer RNAi-kit from Invitrogen. Briefly, the MRP5-DNA sense and antisense templates were used to produce single stranded RNAs (ssRNA) using the BLOCK-iT T7 Enzyme Mix. The ssRNA were combined to dsRNAi. DsRNAi were digested into smaller fragments with dicer enzyme. Human fibroblast cultures were transfected with the dsRNAi fragments with the lipofectamine method. After the days indicated in FIG. 20 the media were harvested and the concentration of hyaluronan was determined by the ELISA reaction.

### Reversal of Osteoarthritic Proteoglycan Loss After Injury

In the following experiment verapamil was given at various time periods after injury to assess, whether the treatment allows rescue of pathological insults.

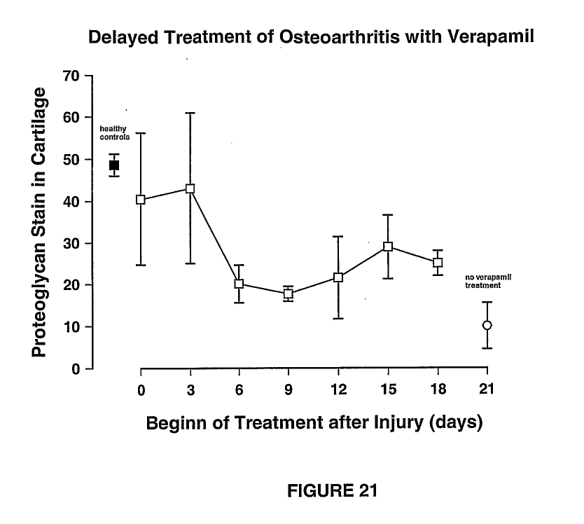

Male Wistar rats (Charles River, Sulzfeld, Germany) having a body weight of 150 g were fed on ordinary laboratory diet and were housed in cages with a floor area of 800 cm². Light-dark cycles were maintained at 12 h/12 h. Room temperature was 21° C. All operations and handling procedures were approved by the district veterinary administration of Muenster and complied with the Animal Protection Act of Germany. The operations were performed under anaesthetic with isofluran/laughing gas. Osteoarthritic damage was induced in the left knees of 6 Wistar rats by injection of a solution of 50 μl of 100 mM iodoacetate into the synovial cavity. The right knees received 50 μl of saline and served as controls. Three rats were feed with normal drinking water and three with drinking water containing 0.75 mg/ml of verapamil. This concentration has been used previously [2]. The rats were given verapamil treatment either immediately or at the times indicated in FIG. 21. Controls were untreated knees and rats that were not treated with verapamil at all. After 21 days, the rats were sacrificed and the articular cartilage was analysed by histology. The kness were fixed in 4% phosphate buffered formalin, decalcified in D-Calcifer (Shandon, Life Sciences International GmbG, Frankfurt, Germany) and embedded in paraffin. Longitudinal sections, 6 μm thick, were cut through the long axis of the tibia in the sagittal plane. Acid polysaccharides were stained with alcian blue and the preparation was counterstained with nuclear fast red. Digital photos were taken and the blue colour of the proteoglycans was quantitatively evaluated by the free Scion Image Software. To eliminate staining artefacts, the staining of proteoglycans on the joint surface was related to the staining density at the growth cone and expressed as percent. Each time point represents the mean of colour reactions obtained from three rats. FIG. 21 shows that verapamil completely protected from proteoglycan loss when administered at the day of injury or three days later. Thereafter verapamil could partially prevent proteoglycan loss up to 21 days after injury (end of the experiments).

### Inhibition of MRP5-Mediated Export by Hyaluronan-Oligosaccharides

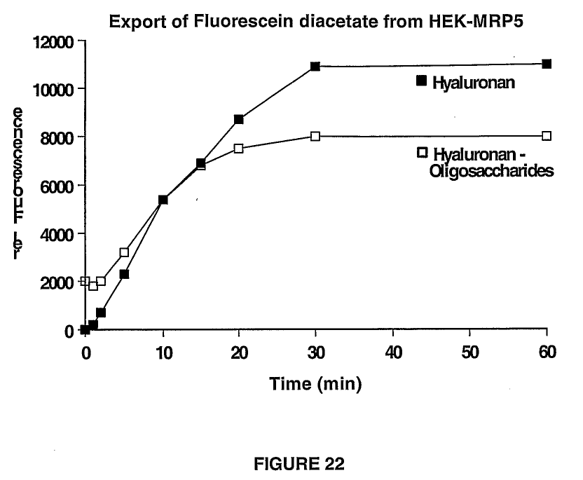

Fluorescein diacetate is a substrate for MRP5-mediated export. Human embryo kidney cells (HEK) overexpressing MRP5 were obtained from Prof. Udo Schurmacher, Universitättsklinik Eppendorf, Hamburg [3]. The cells (2×10⁶/ml) were incubated for 10 min at 4° C. with an equal volume of 4 μM fluorescein diacetate in PBS. Hyaluronan oligosaccharides were prepared by digestion of hyaluronan (Healon) as described [4]. Hyaluronan oligosaccharides (100 μg, mean chain length 6 to 8 disaccharide units as determined by mass spectrometry) or high molecular weight hyaluronan (100 μg) were introduced into the cells by osmotic lysis of pinocytotic vesicles as described previously [5]. The kinetics of export of fluorescein from the cells into the culture medium was measured by the fluorescence spectrometer. FIG. 22 shows that hyaluronan oligosaccharides decreased the export of the MRP5 substrate as compared to high molecular weight hyaluronan, indicating that fluorescein and hyaluronan were exported by the same transporter.

### Inhibition of Endogenous Synthesized Hyaluronan Export by Hyaluronan Oligosaccharides

This experiment was performed to investigate, whether an excess of hyaluronan oligosaccharides introduced into the cytosol interfere with the export of hyaluronan synthesized by the human fibroblasts. Fibroblasts (4×10⁴ cells) were seeded into culture flasks of 25 cm² growth areas and incubated for 24 hours. High molecular weight hyaluronan, hyaluronan oligosaccharides, a mixture of N-acetyl-glucosamine and glucuronic acid or control buffer were introduced into the cytosol by the method of osmotic lysis of pinocytotic vesicles. The concentrations of the added components in the pinocytotic media were 50 μg/ml. The cells were incubated for 40 hours in the presence of [3H]GlcN (8 μCi/ml, specific activity 29 Ci/mmole) and the amount of radioactive hyaluronan released into the culture medium was determined as described [6]. Table 2 shows that hyaluronan oligosaccharides blocked the export of endogenous labelled hyaluronan. This result indicated that excess of cytosolic hyaluronan oligosaccharides blocked the export of endogenously synthesized hyaluronan.

TABLE 2

| | [³H]hyaluronan |
|---|---|
| | cpm |
| buffer | 9.436 |
| Hyaluronan | 10.723 |
| Hyaluronan- | 981 |
| Oligosaccharides | |
| GlcNac/GlcA | 11.881 |

### REFERENCES FOR EXAMPLE 10

[1] Jedlitschky, G., Burchell, B., & Keppler, D. (2000) The multidrug resistance protein 5 functions as an ATP-dependent export pump for cyclic nucleotides. J. Biol. Chem., 275, 30069-30074.
[2] Samnegard, E., Cullen, D. M., Akhter, M. P., & Kimmel, D. B. (2001) No effect of verapamil on the local bone response to in vivo mechanical loading. J. Orthop. Res., 19, 328-336.
[3] Wielinga, P. R., Reid, G., Challa, E. E., Van, D. H., I, van Deemter, L., De Haas, M., Mol, C., Kuil, A. J., Groeneveld, E., Schuetz, J. D., Brouwer, C., De Abreu, R. A., Wijnholds, J., Beijnen, J. H., & Borst, P. (2002) Thiopurine metabolism and identification of the thiopurine metabolites transported by MRP4 and MRP5 overexpressed in human embryonic kidney cells. Mol. Pharmacol., 62, 1321-1331.
[4] Mahoney, D. J., Aplin, R. T., Calabro, A., Hascall, V. C., & Day, A. J. (2001) Novel methods for the preparation and characterization of hyaluronan oligosaccharides of defined length. Glycobiology, 11, 1025-1033.
[5] Prehm, P. (1985) Inhibition of hyaluronate synthesis. Biochem. J., 225, 699-705.
[6] Brecht, M., Mayer, U., Schlosser, E., & Prehm, P. (1986) Increased hyaluronate synthesis is required for fibroblast detachment and mitosis. Biochem. J., 239, 445-450.

### Example 11

### Materials

Hyaluronan was a gift from Genzyme, Cambridge, Mass. Other chemicals were from Sigma Chemical Co, St. Louis, Mo. Hyaluronan oligosaccharides of defined length were prepared be the methods of [1]. Fluorescent hyaluronan was prepared by the Ugi reaction [2]. Briefly, hyaluronan or hyaluronan oligosaccharides (30 mg) were dissolved in 6 ml of a 2:1 mixture of water and dimethylsulfoxide and mixed with a 2 ml solution of 5 mg 5′-aminofluorescein, 10 μl of acetaldehyde and 10 μl cyclohexylisocyanide in dimethylsulfoxide. The suspension was incubated for 3 hours at room temperature. Three volumes of a solution 1.3% potassium acetate in ethanol were added and the precipitate was washed repeatedly with ethanol and dried under vacuum.

### Cells and Cell Culture

Human fibroblasts were grown in suspension culture in Dulbecco's modified Eagles medium supplemented with streptomycin/penicillin (100 units of each/ml) and 10% foetal calf serum. Human embryo kidney cells (HEK) were grown in RPMI-1640 medium supplemented with streptomycin/penicillin (100 units of each/ml) and 10% foetal calf serum. Adherent cells were determined by cell counting after trypsinisation.

### Measurement of Fluorescent Hyaluronan Export

Hyaluronan and other compounds were introduced into the cytosol by osmotic lysis of pinocytotic vesicles [3,4]. The cells were trypsinized and incubated at a cell density of 10⁶ cells/ml with pinocytotic media containing Dulbecco's modified Eagles medium supplemented with streptomycin/penicillin (100 units of each/ml), 10% foetal calf serum, 0.5 M sucrose, 10% polyethylene glycol-6000 and 1 mg/ml of fluorescent hyaluronan or hyaluronan oligosaccharides for 15 min at 37° C. The media were withdrawn and the cells were incubated at 37° C. with DMEM/water (3:2, v/v) for 2 min. The cells were subjected twice to this incubation sequence and then grown further in DMEM. The cells were washed twice with phosphate buffered saline, resuspended in an equal volume of cold phosphate buffered saline containing 5 mM KCl, 1 mM MgCl₂, 5.6 mM glucose and the export of fluorescence was measured in the KC4 fluorescent ELISA reader Synergy HT from BIO-TEK with an excitation at 485 nm and an emission at 528 nm.

### Measurement of Fluorescein Diacetate Export

The export of fluorescein diacetate was monitored by a modified procedure of Rychlik et al. [5]. Non-fluorescent hyaluronan oligosaccharides were introduced into the cytosol of human embryonic kidney cells as described above at a concentration of 1 mg/ml. The cells were then incubated at 4° C. with medium containing 2 mM fluorescein diacetate for 10 min, washed and incubated at 37° C. as described above.

### Inhibition of Endogenous Synthesized Hyaluronan Export by Hyaluronan Oligosaccharides

This experiment was performed to investigate, whether an excess of hyaluronan oligosaccharides introduced into the cytosol interfere with the export of hyaluronan synthesized by the human fibroblasts. Fibroblasts (4×10⁴ cells) were seeded into culture flasks of 25 cm² growth areas and incubated for 24 hours. High molecular weight hyaluronan, hyaluronan oligosaccharides, a mixture of N-acetyl-glucosamine and glucuronic acid or control buffer were introduced into the cytosol by the method of osmotic lysis of pinocytotic vesicles. The concentrations of the added components in the pinocytotic media were 50 μg/ml. The cells were incubated for 40 hours in the presence of [3H]GlcN (8 Ci/ml, specific activity 29 μCi/mmole) and the amount of radioactive hyaluronan released into the culture medium was determined as described [6]. Table 1 shows that hyaluronan oligosaccharides blocked the export of endogenous labelled hyaluronan. This result indicated that excess of cytosolic hyaluronan oligosaccharides blocked the export of endogenously synthesized hyaluronan.

### Determination of Collagen and Proteoglycan Content in Bovine Cartilage Slices

The tissue concentration of collagen was determined by a colour reaction for hydroxyproline after acid hydrolysis as described [7]. Proteoglycans were determined by the method of Bjornsson [8].

### Results

### Export of Fluorescent Hyaluronan Oligosaccharides

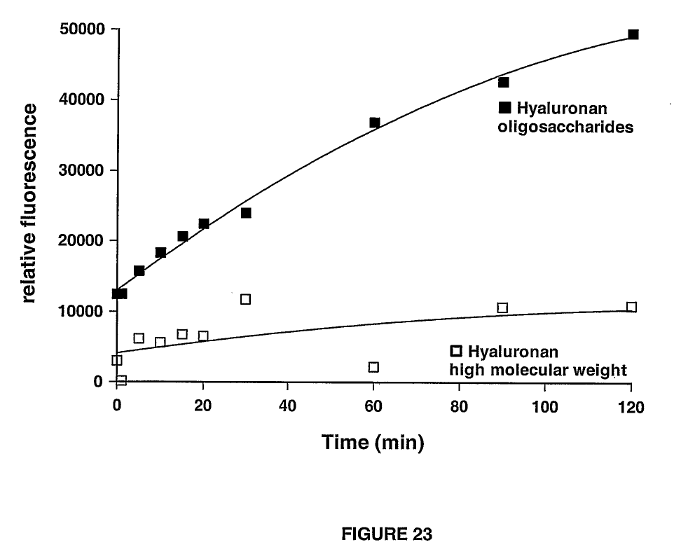

Fluorescent high molecular weight hyaluronan and fluorescent oligosaccharides were introduced into the cytosol by osmotic lysis of pinocytotic vesicles [4]. The kinetics of export was measured in a fluorescent ELISA reader in the presence and absence of the hyaluronan transport inhibitor benzbromaron. FIG. 23 shows that fluorescence appeared in the culture medium only with fluorescent hyaluronan oligosaccharides but not with high molecular weight hyaluronan. This difference suggested that recognition of hyaluronan by the transporter was dependent on the abundant availability of chain termini.

### Intracellular Hyaluronan Oligosaccharides Inhibit the Export of the MRP Substrate Fluorescein Diacetate

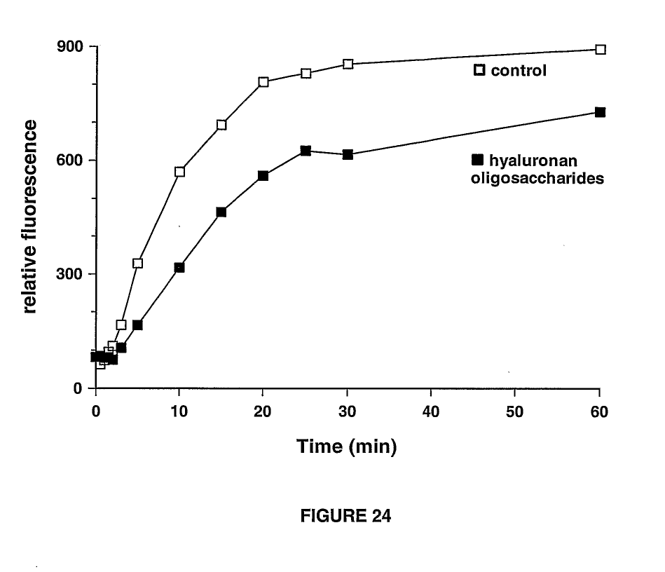

Hyaluronan is exported by MRP5 [9] that also transports fluorescein diacetate [10]. Therefore we analyzed, whether the export of hyaluronan oligosaccharides or high molecular weight hyaluronan interferes with dye transport. FIG. 24 shows that hyaluronan oligosaccharides inhibited dye export. This result suggested that both substrates compete for the same transporter.

### Intracellular Hyaluronan Oligosaccharides Inhibit Endogenous Synthesized Hyaluronan Export

Previous results indicated that hyaluronan synthesis and export was closely coordinated. This experiment was performed to investigate, whether both processes could be uncoupled. Fibroblasts were labelled with [3H]GlcN and hyaluronan oligosaccharides, high molecular weight hyaluronan or an equal mixture of GlcNac and GlcA were introduced into the cytosol. [3H]hyaluronan secreted into the medium was isolated by ion exchange chromatography and the radioactivity was determined. Table 1 shows that only hyaluronan oligosaccharides inhibited cellular hyaluronan production, but not high molecular weight hyaluronan nor a mixture of the monosaccharides. The result suggested that hyaluronan synthesis and export were two independent reactions.

TABLE 1
Inhibition of endogenous synthesized hyaluronan
export by hyaluronan oligosaccharides

| | [³H]hyaluronan |
|---|---|
| | cpm |
| buffer | 9436 |
| Hyaluronan | 10723 |
| Hyaluronan- | 981 |
| Oligosaccharides | |
| GlcNac/GlcA | 11881 |

### Benzbromaron Inhibits Hyaluronan Export

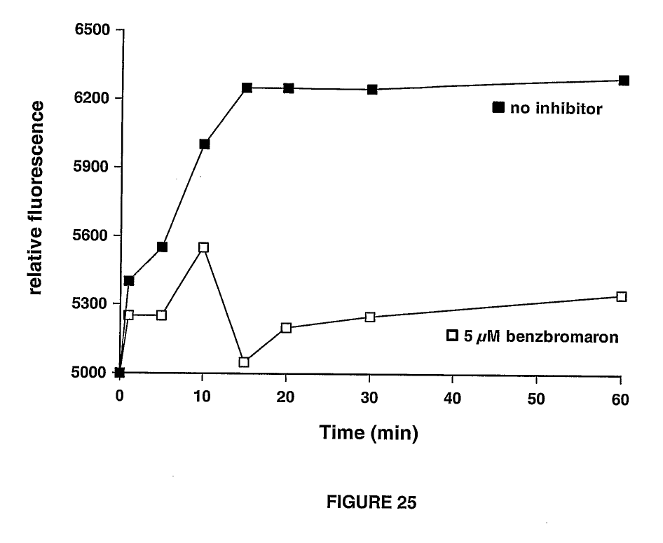

Fluorescent high molecular weight hyaluronan and fluorescent oligosaccharides were introduced into the cytosol by osmotic lysis of pinocytotic vesicles [4]. The kinetics of export was measured in a fluorescent ELISA reader in the presence and absence of the hyaluronan transport inhibitor benzbromaron. FIG. 25 shows that the multidrug resistance transport inhibitor benzbromaron inhibits the export of fluorescent hyaluronan oligosaccharides from the cytosol of human fibroblasts

### Concentration Dependent Inhibition of Proteoglycan Loss

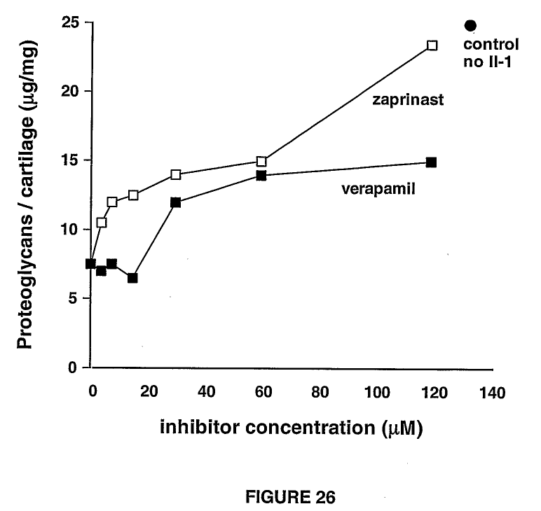

We showed previously that inhibitors of hyaluronan export prevent proteoglycan loss from bovine cartilage. Here we extended these studies to determine the effective concentrations of the inhibitors verapamil and zaprinast. Bovine cartilage was incubated in the absence and presence of 3 ng interleukine-1/ml of media to induce osteoarthritic changes and in the presence of increasing concentrations of verapamil or zaprinast. After 5 days the amount of proteoglycans was determined as described before. FIG. 26 shows the effective concentration range.

### Inhibition of Collagen Degradation by Multidrug Resistance Transport Inhibitor Zaprinast in Bovine Cartilage Slices

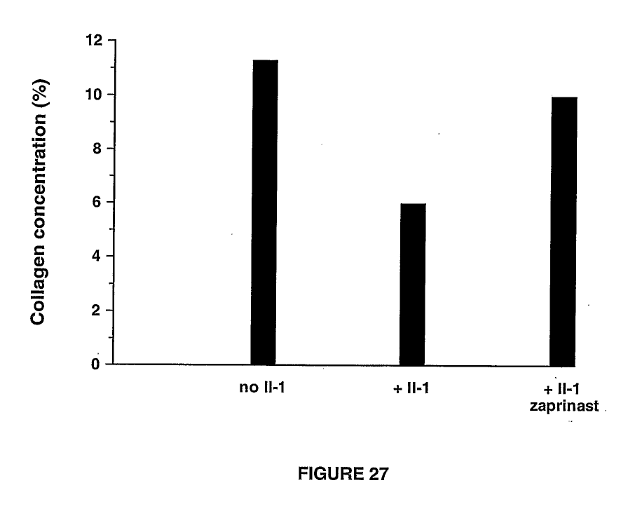

As mentioned earlier, collagen loss from arthritic cartilage is a secondary event after stimulation of hyaluronan production. Therefore we analyzed, whether the loss of collagen can be prevented by inhibitors of hyaluronan export. Bovine cartilage slices were incubated in culture medium in the absence and presence of interleukine-1 to induce arthritic reactions. Hyaluronan export was inhibited by zaprinast. After 21 days the amount of hydroxyproline in acid hydrolysates was determined and the concentration of collagen was calculated from these data. FIG. 27 shows that zaprinast almost reversed the collagen loss induced by II-1.

### REFERENCES FOR EXAMPLE 11

[1] Mahoney, D. J., Aplin, R. T., Calabro, A., Hascall, V. C., & Day, A. J. (2001) Novel methods for the preparation and characterization of hyaluronan oligosaccharides of defined length. Glycobiology, 11, 1025-1033.
[2] Ogamo, A., Matsuzaki, K., Uchiyama, H., & Nagasawa, K. (1982) Preparation and Properties of Fluorescent Glycosaminoglycuronans Labeled with 5-Aminofluorescein. Carbohydrate Research, 105, 69-85.
[3] Okada, C. Y. & Rechsteiner, M. (1982) Introduction of Macromolecules Into Cultured Mammalian-Cells by Osmotic Lysis of Pinocytic Vesicles. Cell, 29, 33-41.
[4] Prehm, P. (1985) Inhibition of hyaluronate synthesis. Biochem. J., 225, 699-705.
[5] Rychlik, B., Balcerczyk, A., Klimczak, A., & Bartosz, G. (2003) The role of multidrug resistance protein 1 (MRP1) in transport of fluorescent anions across the human erythrocyte membrane. J. Membr. Biol., 193, 79-90.
[6] Brecht, M., Mayer, U., Schlosser, E., & Prehm, P. (1986) Increased hyaluronate synthesis is required for fibroblast detachment and mitosis. Biochem. J., 239, 445-450.
[7] STEGEMAN, H. & Staider, K. (1967) Determination of Hydroxyproline. Clinica Chimica Acta, 18, 267-&.
[8] Bjornsson, S. (1993) Simultaneous Preparation and Quantitation of Proteoglycans by Precipitation with Alcian Blue. Anal. Biochem., 210, 282-291.
[9] Prehm, P. & Schumacher, U. (2004) Inhibition of hyaluronan export from human fibroblasts by inhibitors of multidrug resistance transporters. Biochem. Pharmacol., in press.
[10] McAleer, M. A., Breen, M. A., White, N. L., & Matthews, N. (1999) pABC11 (also known as MOAT-C and MRP5), a member of the ABC family of proteins, has anion transporter activity but does not confer multidrug resistance when overexpressed in human embryonic kidney 293 cells. J. Biol. Chem., 274, 23541-23548.
[11] Dreier, R., Grassel, S., Fuchs, S., Schaumburger, J., & Bruckner, P. (2004) Pro-MMP-9 is a specific macrophage product and is activated by osteoarthritic chondrocytes via MMP-3 or a MT1-MMP/MMP-13 cascade. Exp. Cell Res., 297, 303-312.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference.

### REFERENCES

[1] Felson, D. T. & Anderson, J. J. (2002) Hyaluronate sodium injections for osteoarthritis: hope, hype, and hard truths. Arch. Intern. Med., 162, 245-247.
[2] Prehm, P. (1988) Biosynthesis of hyaluronate. Agents Actions, 23, 36-37.
[3] Prehm, P. (1984) Hyaluronate is synthesized at plasma membranes. Biochem. J., 220, 597-600.
[4] Poole, C. A., Matsuoka, A., & Schofield, J. R. (1991) Chondrons from articular cartilage. III. Morphologic changes in the cellular microenvironment of chondrons isolated from osteoarthritic cartilage. Arthritis Rheum., 34, 22-35.
[5] Hamerman, D., Sasse, J., & Klagsbrun, M. (1986) A cartilage-derived growth factor enhances hyaluronate synthesis and diminishes sulfated glycosaminoglycan synthesis in chondrocytes. J. Cell Physiol., 127, 317-322.
[6] Bourguignon, L. Y., Singleton, P. A., Zhu, H., & Zhou, B. (2002) Hyaluronan Promotes Signaling Interaction between CD44 and the Transforming Growth Factor beta Receptor I in Metastatic Breast Tumor Cells. J. Biol. Chem., 277, 39703-39712.
[7] Fujita, Y., Kitagawa, M., Nakamura, S., Azuma, K., Ishii, G., Higashi, M., Kishi, H., Hiwasa, T., Koda, K., Nakajima, N., & Harigaya, K. (2002) CD44 signaling through focal adhesion kinase and its anti-apoptotic effect. FEBS Lett., 528, 101.
[8] Suzuki, M., Kobayashi, H., Kanayama, N., Nishida, T., Takigawa, M., & Terao, T. (2002) CD44 stimulation by fragmented hyaluronic acid induces upregulation and tyrosine phosphorylation of c-Met receptor protein in human chondrosarcoma cells. Biochim. Biophys. Acta, 1591, 37.
[9] Knudson, C. B., Nofal, G. A., Pamintuan, L., & Aguiar, D. J. (1999) The chondrocyte pericellular matrix: a model for hyaluronan-mediated cell-matrix interactions. Biochem. Soc. Trans., 27, 142-147.
[10] Chow, G., Nieffeld, J. J., Knudson, C. B., & Knudson, W. (1998) Antisense inhibition of chondrocyte CD44 expression leading to cartilage chondrolysis. Arthritis Rheum., 41, 1411-1419.
[11] Ng, C. K., Handley, C. J., Preston, B. N., & Robinson, H. C. (1992) The extracellular processing and catabolism of hyaluronan incultured adult articular cartilage explants. Arch. Biochem. Biophys., 298, 70-79.
[12] Morales, T. I. & Hascall, V. C. (1988) Correlated metabolism of proteoglycans and hyaluronic acidin bovine cartilage organ cultures. J. Biol. Chem., 263, 3632-3638.
[13] Jiang, H., Peterson, R. S., Wang, W., Bartnik, E., Knudson, C. B., & Knudson, W. (2002) Requirement for the CD44 cytoplasmic domain for Hyaluronan binding, pericellularmatrix assembly, and receptor-mediated endocytosis in COS-7 cells. J. Biol. Chem.
[14] D'Souza, A. L., Masuda, K., Otten, L. M., Nishida, Y., Knudson, W., & Thonar, E. J. (2000) Differential Effects of Interleukin-1 on Hyaluronan and Proteoglycan Metabolism in Two Compartments of the Matrix Formed by Articular Chondrocytes Maintained in Alginate. Arch. Biochem. Biophys., 374, 59-65.
[15] Nishida, Y., D'Souza, A. L., Thonar, E. J., & Knudson, W. (2000) Stimulation of hyaluronan metabolism by interleukin-1 alpha in human articular cartilage. Arthritis Rheum., 43, 1315-1326.
[16] Kozaci, L. D., Buttle, D. J., & Hollander, A. P. (1997) Degradation of type II collagen, but not proteoglycan, correlates with matrix metalloproteinase activity in cartilage explant cultures. Arthritis Rheum., 40, 164-174.
[17] Billinghurst, R. C., Wu, W., Ionescu, M., Reiner, A., Dahlberg, L., Chen, J., van Wart, H., & Poole, A. R. (2000) Comparison of the degradation of type II collagen and proteoglycan in nasal and articular cartilages induced by interleukin-1 and the selective inhibition of type II collagen cleavage by collagenase. Arthritis Rheum., 43, 664-672.
[18] Kozaci, L. D., Brown, C. J., Adcocks, C., Galloway, A., Hollander, A. P., & Buttle, D. J. (1998) Stromelysin 1, neutrophil collagenase, and collagenase 3 do not play major roles in a model of chondrocyte mediated cartilage breakdown. Mol. Pathol., 51, 282-286.
[19] Knudson, W., Casey, B., Nishida, Y., Eger, W., Kuettner, K. E., & Knudson, C. B. (2000) Hyaluronan oligosaccharides perturb cartilage matrix homeostasis and induce chondrocytic chondrolysis. Arthritis Rheum., 43, 1165-1174.
[20] Janusz, M. J., Bendele, A. M., Brown, K. K., Taiwo, Y. O., Hsieh, L., & Heitmeyer, S. A. (2002) Induction of osteoarthritis in the rat by surgical tear of the meniscus: Inhibition of joint damage by a matrix metalloproteinase inhibitor. Osteoarthritis. Cartilage., 10, 785-791.
[21] Greenwald, R. A. (1999) Thirty-six years in the clinic without an MMP inhibitor. What hath collagenase wrought? Ann. N.Y. Acad. Sci., 878, 413-419.
[22] Prehm, P. (1983) Synthesis of hyaluronate in differentiated teratocarcinoma cells. Mechanism of chain growth. Biochem. J., 211, 191-198.
[23] Prehm, P. (1983) Synthesis of hyaluronate in differentiated teratocarcinoma cells. Characterization of the synthase. Biochem. J., 211, 181-189.
[24] Prehm, P. Novel nucleotide-sugar derivatives. DE19843428976 19840806-[DE3428976]. 1986. Ref Type: Patent
[25] Prehm, P. (1985) Inhibition of hyaluronate synthesis. Biochem. J., 225, 699-705.
[26] Ueki, N., Taguchi, T., Takahashi, M., Adachi, M., Ohkawa, T., Amuro, Y., Hada, T., & Higashino, K. (2000) Inhibition of hyaluronan synthesis by vesnarinone in cultured human myofibroblasts. Biochim. Biophys. Acta, 1495, 160-167.
[27] Nakamura, T., Funahashi, M., Takagaki, K., Munakata, H., Tanaka, K., Saito, Y., & Endo, M. (1997) Effect of 4-methylumbelliferone on cell-free synthesis of hyaluronic acid. Biochem. Mol. Biol. Int., 43, 263-268.
[28] Nakamura, T., Takagaki, K., Shibata, S., Tanaka, K., Higuchi, T., & Endo, M. (1995) Hyaluronic-acid-deficient extracellular matrix induced by addition of 4-methylumbelliferone to the medium of cultured human skin fibroblasts. Biochem. Biophys. Res. Commun., 208, 470-475.
[29] Kakizaki, I., Takagaki, K., Endo, Y., Kudo, D., Ikeya, H., Miyoshi, T., Baggenstoss, B. A., Tlapak-Simmons, V. L., Kumari, K., Nakane, A., Weigel, P. H., & Endo, M. (2002) Inhibition of hyaluronan synthesis in Streptococcus equi FM100 by 4-methylumbelliferone. Eur. J. Biochem., 269, 5066-5075.
[30] Klewes, L. & Prehm, P. (1994) Intracellular signal transduction for serum activation of the hyaluronan synthase in eukaryotic cell lines. J. Cell Physiol., 160, 539-544.
[31] Prehm, P. (2002) Hyaluronan. In Biopolymers (Steinbüchel, A., ed), pp. 379-406. Whiley-VCH-Verlag, Weinheim, Germany.
[32] Altman, R. D. & Moskowitz, R. (1998) Intraarticular sodium hyaluronate (Hyalgan(R)) in the treatment of patients with osteoarthritis of the knee: A randomized clinical trial. J. Rheumatol., 25, 2203-2212.
[33] Adams, M. E., Lussier, A. J., & Peyron, J. G. (2000) A risk-benefit assessment of injections of hyaluronan and its derivatives in the treatment of osteoarthritis of the knee. Drug Saf, 23, 115-130.
[34] Wobig, M., Bach, G., Beks, P., Dickhut, A., Runzheimer, J., Schwieger, G., Vetter, G., & Balazs, E. A. (1999) The role of elastoviscosity in the efficacy of viscosupplementation for osteoarthritis of the knee: A comparison of hylan G-F 20 and a lower-molecular-weight hyaluronan. Clin. Ther., 21, 1549-1562.
[35] Scott, J. E., Thomlinson, A. M., & Prehm, P. (2003) Supramolecular organization in streptococcal pericellular capsules is based on hyaluronan tertiary structures. Exp. Cell Res., 285, 1-8.
[36] Kass, E. H. & Seastone, C. V. (1944) The role of the mucoid polysaccharide hyaluronic acid in the virulence of group A hemolytic streptococci. J. Exp. Med., 70, 319-330.
[37] Wessels, M. R., Moses, A. E., Goldberg, J. B., & DiCesare, T. J. (1991) Hyaluronic acid capsule is a virulence factor for mucoid group A streptococci. Proc. Natl. Acad. Sci. U.S.A., 88, 8317-8321.
[38] Wessels, M. R., Goldberg, J. B., Moses, A. E., & DiCesare, T. J. (1994) Effects on virulence of mutations in a locus essential for hyaluronic acid capsule expression in group A streptococci. Infect Immun., 62, 433-441.
[39] Whitnack, E., Bisno, A. L., & Beachey, E. H. (1981) Hyaluronate capsule prevents attachment of group A streptococci to mouse peritoneal macrophages. Infect. Immun., 31, 985-991.
[40] Cleary, P. P. & Larkin, A. (1979) Hyaluronic acid capsule: strategyforoxygen resistance in group A streptococci. J. Bacteriol., 140, 1090-1097.
[41] Nickel, V., Prehm, S., Lansing, M., Mausolf, A., Podbielski, A., Deutscher, J., & Prehm, P. (1998) An ectoprotein kinase of group C streptococci binds hyaluronan and regulates capsule formation. J. Biol. Chem., 273, 23668-23673.
[42] Prehm, S., Nickel, V., & Prehm, P. (1996) A mild purification method for polysaccharide binding membrane proteins: phase separation of digitonin extracts to isolate the hyaluronate synthase from Streptococcus sp. in active form. Protein Expression and Purification, 7, 343-346.
[43] Ashbaugh, C. D. & Wessels, M. R. (1995) Identification of a gene similar to ABC transporters near the capsule synthesis region of the group A streptococcal chromosome. Dev. Biol. Stand., 85, 231-235.
[44] Ashbaugh, C. D., Alberti, S., & Wessels, M. R. (1998) Molecular analysis of the capsule gene region of group A Streptococcus: the hasAB genes are sufficient for capsule expression. J. Bacteriol., 180, 4955-4959.
[45] Tlapak-Simmons, V. L., Kempner, E. S., Baggenstoss, B. A., & Weigel, P. H. (1998) The active streptococcal hyaluronan synthases (HASs) contain a single HAS monomer and multiple cardiolipin molecules. J. Biol. Chem., 273, 26100-26109.
[46] DeAngelis, P. L., Papaconstantinou, J., & Weigel, P. H. (1993) Molecular cloning, identification, and sequence of the hyaluronan synthase gene from group A Streptococcus pyogenes. J. Biol. Chem., 268, 19181-19184.
[47] Paulsen, I. T., Benness, A. M., & Saier, M. H. J. (1997) Computer-based analysis of protein constitutents of transport systems catalysing export of complex carbohydrates in bacteria. Microbiology., 143, 2685-2699.
[48] Robert, J. (1999) Multidrug resistance in oncology: diagnostic and therapeutic approaches. Eur. J. Clin. Invest, 29, 536-545.
[49] Krishna, R. & Mayer, L. D. (2000) Multidrug resistance (MDR) in cancer. Mechanisms, reversal using modulators of MDR and the role of MDR modulators in influencing the pharmacokinetics of anticancer drugs. Eur. J. Pharm. Sci., 11, 265-283.
[50] Hollo, Z., Homolya, L., Hegedus, T., & Sarkadi, B. (1996) Transport properties of the multidrug resistance-associated protein (MRP) in human tumour cells. FEBS Lett., 383, 99-104.
[51] Becq, F., Hamon, Y., Bajetto, A., Gola, M., Verrier, B., & Chimini, G. (1997) ABC1, an ATP binding cassette transporter required for phagocytosis of apoptotic cells, generates a regulated anion flux after expression in Xenopus laevis oocytes. J. Biol. Chem., 272, 2695-2699.
[52] von Eckardstein, A., Langer, C., Engel, T., Schaukal, I., Cignarella, A., Reinhardt, J., Lorkowski, S., Li, Z., Zhou, X., Cullen, P., & Assmann, G. (2001) ATP binding cassette transporter ABCA1 modulates the secretion of apolipoprotein E from human monocyte-derived macrophages. FASEB J., 15, 1555-1561.
[53] Wiese, M. & Pajeva, I. K. (2001) Structure-activity relationships of multidrug resistance reversers. Curr. Med. Chem., 8, 685-713.
[54] Schinkel, A. H. & Jonker, J. W. (2003) Mammalian drug efflux transporters of the ATP binding cassette (ABC) family: an overview. Adv. Drug Deliv. Rev., 55, 3-29.
[55] Filep, J. G., Skrobik, Y., Fournier, A., & Foldes-Filep, E. (1996) Effects of calcium antagonists on endothelin-1-induced myocardial ischaemia and oedema in the rat. Br. J. Pharmacol., 118, 893-900.
[56] Taherzadeh, M. & Warren, J. B. (1997) Comparison of diltiazem and verapamil on rat microvascular permeability. Microvasc. Res., 54, 206-213.
[57] Taherzadeh, M., Das, A. K., & Warren, J. B. (1998) Nifedipine increases microvascular permeability via a direct local effect on postcapillary venules. Am. J. Physiol, 275, H1388-H1394.
[58] van Zuylen, L., Nooter, K., Sparreboom, A., & Verweij, J. (2000) Development of multidrug-resistance convertors: sense or nonsense? Invest New Drugs, 18, 205-220.
[59] Oyanagui, Y. (1998) Immunosuppressants enhance superoxide radical/nitric oxide-dependent dexamethasone suppression of ischemic paw edema in mice. Eur. J. Pharmacol., 344, 241-249.
[60] Munkonge, F. M., Osborne, L. R., Geddes, D. M., & Alton, E. W. (1994) Lack of inhibition by dideoxy-forskolin and verapamil of DIDS-sensitive volume-activated Cl-secretion in human squamous lung carcinoma epithelial cells. Biochim. Biophys. Acta, 1224, 342-348.
[61] Pohl, A., Lage, H., Muller, P., Pomorski, T., & Herrmann, A. (2002) Transport of phosphatidylserine via MDR1 (multidrug resistance 1) P-glycoprotein in a human gastric carcinoma cell line. Biochem. J, 365, 259-268.
[62] Berger, W., Hauptmann, E., Elbling, L., Vetterlein, M., Kokoschka, E. M., & Micksche, M. (1997) Possible role of the multidrug resistance-associated protein (MRP) in chemoresistance of human melanoma cells. Int J. Cancer, 71, 108-115.
[63] Misra, S., Ghatak, S., Zoltan-Jones, A., & Toole, B. P. (2003) Regulation of multi-drug resistance in cancer cells by hyaluronan. J. Biol. Chem.
[64] Maguin, E., Prevost, H., Ehrlich, S. D., & Gruss, A. (1996) Efficient insertional mutagenesis in lactococci and other gram-positive bacteria. J. Bacteriol., 178, 931-935.
[65] Wilson, A. T. (1959) The relative importance of the capsule and the M-antigen in determining colony form of group A streptococci. J. Exp. Med., 109, 257-269.
[66] Levin, J. C. & Wessels, M. R. (1998) Identification of csrR/csrS, a genetic locus that regulates hyaluronic acid capsule synthesis in group A Streptococcus. Mol. Microbiol., 30, 209-219.
[67] Bernish, B. & van de Rijn, I. (1999) Characterization of a two-component system in Streptococcus pyogenes which is involved in regulation of hyaluronic acid production. J. Biol. Chem., 274, 4786-4793.
[68] Gryllos, I., Levin, J. C., & Wessels, M. R. (2003) The CsrR/CsrS two-component system of group A Streptococcus responds to environmental Mg2+. Proc. Natl. Acad. Sci. U.S.A.
[69] Brecht, M., Mayer, U., Schlosser, E., & Prehm, P. (1986) Increased hyaluronate synthesis is required for fibroblast detachment and mitosis. Biochem. J., 239, 445-450.
[70] Rao, V. V., Anthony, D. C., & Piwnica-Worms, D. (1994) MDR1 gene-specific monoclonal antibody C494 cross-reacts with pyruvate carboxylase. Cancer Res., 54, 1536-1541.
[71] Sela, S., Husain, S. R., Pearson, J. W., Longo, D. L., & Rahman, A. (1995) Reversal of multidrug resistance in human colon cancer cells expressing the human MDR1 gene by liposomes in combination with monoclonal antibody or verapamil. J. Natl. Cancer Inst., 87, 123-128.
[72] Lorke, D. E., Kruger, M., Buchert, R., Bohuslavizki, K. H., Clausen, M., & Schumacher, U. (2001) In vitro and in vivo tracer characteristics of an established multidrug-resistant human colon cancer cell line. J. Nucl. Med., 42, 646-654.
[73] Robbins, J. R., Thomas, B., Tan, L., Choy, B., Arbiser, J. L., Berenbaum, F., & Goldring, M. B. (2000) Immortalized human adult articular chondrocytes maintain cartilage-specific phenotype and responses to interleukin-1 beta. Arthritis Rheum., 43, 2189-2201.
[74] Fosang, A. J., Hey, N. J., Carney, S. L., & Hardingham, T. E. (1990) An ELISA plate-based assay for hyaluronan using biotinylated proteoglycan G1 domain (HA-binding region). Matrix, 10, 306-313.
[75] Chandrasekhar, S., Esterman, M. A., & Hoffman, H. A. (1987) Microdetermination of proteoglycans and glycosaminoglycans in the presence of guanidine hydrochloride. Anal. Biochem., 161, 103-108.
[76] Terry, D. E., Chopra, R. K., Ovenden, J., & Anastassiades, T. P. (2000) Differential use of Alcian blue and toluidine blue dyes for the quantification and isolation of anionic glycoconjugates from cell cultures: application to proteoglycans and a high-molecular-weight glycoprotein synthesized by articular chondrocytes. Anal. Biochem., 285, 211-219.
[77] Guingamp, C., Gegout-Pottie, P., Philippe, L., Terlain, B., Netter, P., & Gillet, P. (1997) Mono-iodoacetate-induced experimental osteoarthritis: a dose-response study of loss of mobility, morphology, and biochemistry. Arthritis Rheum., 40, 1670-1679.
[78] Duwat, P., Cochu, A., Ehrlich, S. D., & Gruss, A. (1997) Characterization of Lactococcus lactis UV-sensitive mutants obtained by ISS1 transposition. J. Bacteriol., 179, 4473-4479.
[79] Trieu-Cuot, P., Carlier, C., Poyart-Salmeron, C., & Courvalin, P. (1990) A pair of mobilizable shuttle vectors conferring resistance to spectinomycin for molecular cloning in Escherichia coli and in gram-positive bacteria. Nucleic Acids Res., 18, 4296.
[80] Sambrook, J., Fritsch, E. F., & Maniatis, T. (1989) Molecular cloning—a laboratory manual, 2 edn. Cold Spring Harbor Laboratory Press, Cold Spring Harbor.
[81] Dower, W. J., Miller, J. F., & Ragsdale, C. W. (1988) High efficiency transformation of E. coli by high voltage electroporation. Nucleic Acids Res., 16, 6127-6145.
[82] O'Connor, S. P. & Cleary, P. P. (1987) In vivo Streptococcus pyogenes C5a peptidase activity: analysis using transposon- and nitrosoguanidine-induced mutants. J. Infect. Dis., 156, 495-504.
[83] Simon, D. & Ferretti, J. J. (1991) Electrotransformation of Streptococcus pyogenes with plasmid and linear DNA. FEMS Microbiol. Lett., 66, 219-224.
[84] Ochman, H., Medhora, M. M., Garza, D., & Hartl, D. L. (1989) Amplification of flanking sequences by inverse PCR. In PCR protocols. A guide to methods and applications (Innis, M. A., Gelfand, D. H., Sninsky, J. J., & White, T. J., eds), pp. 219-227. Academic Press, New York.
[85] Schrager, H. M., Rheinwald, J. G., &Wessels, M. R. (1996) Hyaluronicacid capsule and the role of streptococcal entry into keratinocytes in invasive skin infection. J. Clin. Invest, 98, 1954-1958.
[86] Dube, B., Luke, H. J., Aumailley, M., & Prehm, P. (2001) Hyaluronan reduces migration and proliferation in CHO cells. Biochim. Biophys. Acta, 1538, 283-289.
[87] Stern, M. & Stern, R. (1992) An ELISA-like assay for hyaluronidase and hyaluronidaseinhibitors. Matrix, 12, 397-403.
[88] Samnegard, E., Cullen, D. M., Akhter, M. P., & Kimmel, D. B. (2001) No effect of verapamil on the local bone response to in vivo mechanical loading. J. Orthop. Res., 19, 328-336.
[89] Lüke, H. J. & Prehm, P. (1999) Synthesis and shedding of hyaluronan from plasma membranes of human fibroblasts and metastatic and non-metastatic melanoma cells. Biochem. J., 343, 71-75.
[90] Dieppe, P. & Lim, K. (1998) Osteoarthritis and related disorders. Clinical features and diagnostic problems. In Rheumatology (Klippel, J. H. & Dieppe, P. A., eds), pp. 3.1-3.16. Mosby, London.
[91] Alberts, B., Bray, D., Lewis, J., Raff, M., Roberts, K., & Watson, J. D. (2002) Molecular Biology of the Cell, 3 edn. Garland, N.Y.
[92] Stein, W. D. (1997) Kinetics of the multidrug transporter (P-glycoprotein) and its reversal. Physiol Rev., 77, 545-590.
[93] Twentyman, P. R., Rhodes, T., & Rayner, S. (1994) A comparison of rhodamine 123 accumulation and efflux in cells with P-glycoprotein-mediated and MRP-associated multidrug resistance phenotypes. Eur. J. Cancer, 30A, 1360-1369.
[94] Mangham, D. C., Cannon, A., Komiya, S., Gendron, R. L., Dunussi, K., Gebhardt, M. C., Mankin, H. J., & Arceci, R. J. (1996) P-glycoprotein is expressed in the mineralizing regions of the skeleton. Calcif Tissue Int., 58, 186-191.
[95] Rosier, R. N., O'Keefe, R. J., Teot, L. A., Fox, E. J., Nester, T. A., Puzas, J. E., Reynolds, P. R., & Hicks, D. G. (1997) P-glycoprotein expression in cartilaginous tumors. J. Surg. Oncol., 65, 95-105.
[96] Cooper, C. (1998) Osteoarthritis and related disorders. Epidemiology. In Rheumatology (Klippel, J. H. & Dieppe, P. A., eds), pp. 2.1-2.8. Mosby, London.
[97] Brecht, M., Mayer, U., Schlosser, E., & Prehm, P. (1986) Increased hyaluronate synthesis is required for fibroblast detachment and mitosis. Biochem. J., 239, 445-450.
[98] Bitter, T. & Muir, H. M. (1960) A modified uronic acid carbazol reaction. Anal. Biochem., 4, 330-334.
[99] Stern, M. & Stern, R. (1992) An ELISA-like assay for hyaluronidase and hyaluronidaseinhibitors. Matrix, 12, 397-403.
[100] de Belder, A. N. & Wik, K. O. (1975) Preparation and properties of fluorescein-labelled hyaluronate. Carbohydr. Res., 44, 251-257.
[101] Rao, C. M., Jilani, A., Swarnakar, S., Deb, T. B., & Datta, K. (1996) A method to radioiodinate hyaluronic acid and its use as a probe to detect hyaluronic acid-binding proteins. J. Biol. Chem., 255, 7218-7224.
[102] Termeer, C. C., Hennies, J., Voith, U., Ahrens, T., Weiss, M., Prehm, P., & Simon, J. C. (2000) Oligosaccharides of hyaluronan are potent activators of dendritic cells. J. Immunol., 165, 1863-1870.
[103] Prehm, P. & Scheid, A. (1978) Sensitive method for the analysis of carbohydrates by gas chromatography of 3H-labeled alditol acetates. J. Chromatogr., 166, 461-467.
[104] Beck, J. F., Buchholz, F., Ulrich, W. R., Boer, R., Sanders, K. H., Niethammer, D., & Gekeler, V. (1998) Rhodamine 123 efflux modulation in the presence of low or high serum from CD56+ hematopoietic cells or CD34+ leukemic blasts by B9309-068, a newly designed pyridine derivative. Cancer Lett., 129, 157-163.
[105] Naito H, Ziegler M M, Miyakawa A, Tokunaga O, & Sasaki M (1996) Establishment of animal liver metastatic model for C-1300 murine neuroblastoma and immunotherapy for it using OK-432, streptococcus preparation. J-Surg-Res. 1992 January; 52(1): 79-84, -84.
[106] Felgner, P. L., Tsai, Y. J., Sukhu, L., Wheeler, C. J., Manthorpe, M., Marshall, J., & Cheng, S. H. (1995) Improved cationic lipid formulations for in vivo gene therapy. Ann. N.Y. Acad. Sci., 772, 126-139.
[107] McCarthy, M. T. & Toole, B. P. (1989) Membrane-associated hyaluronate-binding activity of chondrosarcoma chondrocytes. J. Cell Physiol., 141, 191-202.
[108] Bansal, M. K. & Mason, R. M. (1986) Evidence for rapid metabolic turnover of hyaluronate synthetase in Swarm rat chondrosarcoma chondrocytes. Biochem. J., 236, 515-519.
[109] Mason, R. M., Kimura, J. H., & Hascall, V. C. (1982) Biosynthesis of hyaluronic acid in cultures of chondrocytes from the Swarm rat chondrosarcoma. J. Biol. Chem., 257, 2236-2245.
[110] Wyman, J. J., Hornstein, A. M., Meitner, P. A., Mak, S., Verdier, P., Block, J. A., Pan, J., & Terek, R. M. (1999) Multidrug resistance-1 and p-glycoprotein in human chondrosarcoma cell lines: expression correlates with decreased intracellular doxorubicin and in vitro chemoresistance. J. Orthop. Res., 17, 935-940.
[111] Terek, R. M., Schwartz, G. K., Devaney, K., Glantz, L., Mak, S., Healey, J. H., & Albino, A. P. (1998) Chemotherapy and P-glycoprotein expression in chondrosarcoma. J. Orthop. Res., 16, 585-590.
[112] Rosier, R. N., O'Keefe, R. J., Teot, L. A., Fox, E. J., Nester, T. A., Puzas, J. E., Reynolds, P. R., & Hicks, D. G. (1997) P-glycoprotein expression in cartilaginous tumors. J. Surg. Oncol., 65, 95-105.
[113] Beck, J. F., Buchholz, F., Ulrich, W. R., Boer, R., Sanders, K. H., Niethammer, D., & Gekeler, V. (1998) Rhodamine 123 efflux modulation in the presence of low or high serum from CD56+ hematopoietic cells or CD34+ leukemic blasts by B9309-068, a newly designed pyridine derivative. Cancer Lett., 129, 157-163.
[114] Naito, S., Koike, K., Ono, M., Machida, T., Tasaka, S., Kiue, A., Koga, H., & Kumazawa, J. Development of novel reversal agents, imidazothiazole derivatives, targeting MDR1- and MRP-mediated multidrug resistance. Oncol. Res. 10, 123-132. 1996.
[115] Collis, L., Hall, C., Lange, L., Ziebell, M., Prestwich, R., & Turley, E. A. (1998) Rapid hyaluronan uptake is associated with enhanced motility: implications for an intracellular mode of action. FEBS LETTERS., 440, 444-449.
[116] Felgner, P. L., Tsai, Y. J., Sukhu, L., Wheeler, C. J., Manthorpe, M., Marshall, J., & Cheng, S. H. (1995) Improved cationic lipid formulations for in vivo gene therapy. Ann. N.Y. Acad. Sci., 772, 126-139.
[117] Asplund, T., Versnel, M. A., Laurent, T. C., & Heldin, P. (1993) Human mesothelioma cells produce factors that stimulate the production of hyaluronan by mesothelial cells and fibroblasts. Cancer Res., 53, 388-392.
[118] Jacobson, A., Rahmanian, M., Rubin, K., & Heldin, P. (2002) Expression of hyaluronan synthase 2 or hyaluronidase 1 differentially affect the growth rate of transplantable colon carcinoma cell tumors. Int. J. Cancer, 102, 212-219.
[119] Tufveson, G., Haligren, R., Johnsson, C., & Wahlberg, J. Use of hyaluronidase in treatment of interstitial edema from organ grafts. Tufveson, Gunnar, Hallgren, Roger, Johnsson, Cecilia, and Wahlberg, Jan. [VO 9808538],
[120] Engstrom Laurent, A., Feltelius, N., Haligren, R., &Wasteson, A. (1985) Raised serum hyaluronate levels in scleroderma: an effectof growth factor induced activation of connective tissuecells? Ann. Rheum. Dis., 44, 614-620.
[121] Lundin, A., Engstrom Laurent, A., Haligren, R., & Michaelsson, G. (1985) Circulating hyaluronate in psoriasis. Br. J. Dermatol., 112, 663-671.
[122] Haligren, R., Eklund, A., Engstrom Laurent, A., & Schmekel, B. (1985) Hyaluronate in bronchoalveolar lavage fluid: a new markerin sarcoidosis reflecting pulmonary disease. Br. Med. J. Clin. Res. Ed., 290, 1778-1781.
[123] Eklund, A., Hallgren, R., Blaschke, E., Engstrom Laurent, A. P.-U., & Svane, B. (1987) Hyaluronate in bronchoalveolar lavage fluid in sarcoidosisand its relationship to alveolar cell populations. Eur. J. Respir. Dis., 71, 30-36.
[124] Nettelbladt, O. & Haligren, R. (1989) Hyaluronan (hyaluronic acid) in bronchoalveolar lavage fluid during the development of bleomycin-induced alveolitis in therat. Am. Rev. Respir. Dis., 140, 1028-1032.
[125] Nettelbladt, O., Lundberg, K., Tengblad, A., & Hallgren, R. (1990) Accumulation of hyaluronan in bronchoalveolar lavage fluidis independent of iron-, complement- and granulocyte-depletionin bleomycin-induced alveolitis in the rat. Eur. Respir. J., 3, 765-771.
[126] Haligren, R., Gerdin, B., Tengblad, A., & Tufveson, G. (1990) Accumulation of hyaluronan (hyaluronic acid) in myocardial interstitial tissue parallels development of transplantation edema in heart allografts in rats. J. Clin. Invest, 85, 668-673.
[127] Nettelbladt, O., Scheynius, A., Bergh, J., Tengblad, A., & Haligren, R. (1991) Alveolar accumulation of hyaluronan and alveolar cellularresponse in bleomycin-induced alveolitis. Eur. Respir. J., 4, 407-414.
[128] Bjermer, L., Haligren, R., Nilsson, K., Franzen, L., Sandstrom, T. S.-B., & Henriksson, R. (1992) Radiation-induced increase in hyaluronan and fibronectin inbronchoalveolar lavage fluid from breast cancer patients issuppressed by smoking. Eur. Respir. J., 5, 785-790.
[129] Ahrenstedt, O., Knutson, L., Haligren, R., & Gerdin, B. (1992) Increased luminal release of hyaluronan in uninvolvedjejunum in active Crohn's disease but not in inactive disease orin relatives. Digestion, 52, 6-12.
[130] Johnsson, C., Haligren, R., & Tufveson, G. (1993) Recovery of hyaluronan during perfusion of small bowel transplantation reflects rejection. Transplantation, 55, 477-479.
[131] Waldenstrom, A., Fohiman, J., Iback, N. G., Ronquist, G., & Haligren, R. G.-B.

(1993) Coxsackie B3 myocarditis induces a decrease in energy charge and accumulation of hyaluronan in the mouse heart. Eur. J. Clin. Invest., 23, 277-282.
[132] Wells, A., Larsson, E., Hanas, E., Laurent, T., & Hallgren, R. T.-G. (1993) Increased hyaluronan in acutely rejecting human kidney grafts. Transplantation, 55, 1346-1349.
[133] Tufveson, G., Hallgren, R., Johnsson, C., & Wahlberg, J. Use ofhyaluronidase in treatment of interstitial edema from organ grafts. Tufveson, Gunnar, Haligren, Roger, Johnsson, Cecilia, and Wahlberg, Jan. WO 97-SE1313-[WO 9808538 A1]. 1998. PCT Int. Appl., 18 pp. CODEN: PIXXD2. 24-7-1997. Ref Type: Patent
[134] Johnsson, C., Haligren, R., Elvin, A., Gerdin, B., & Tufveson, G. (1999) Hyaluronidase ameliorates rejection-induced edema. TRANSPLANT. INTERNATIONAL., 12, 235-243.
[135] Johnsson, C., Hallgren, R., & Tufveson, G. (2000) Role of hyaluronan in acute pancreatitis. Surgery, 127, 650-658.
[136] Kimata, K., Honma, Y., Okayama, M., Oguri, K., Hozumi, M., & Suzuki, S. (1983) Increased synthesis of hyaluronic acid by mouse mammarycarcinoma cell variants with high metastatic potential. Cancer Res., 43, 1347-1354.
[137] Zhang, L., Underhill, C. B., & Chen, L. (1995) Hyaluronan on the surface of tumor cells is correlated with metastatic behavior. Cancer Res., 55, 428-433.
[138] West, D. C. & Shaw, D. M. (1998) Tumour hyaluronan in relation to angiogenesis and metastasis. In The chemistry, biology and medical applications of hyaluronan and its derivatives (Laurent, T. C., ed), pp. 227-233. Portland Press, London.
[139] Simpson, M. A., Reiland, J., Burger, S. R., Furcht, L. T., Spicer, A. P., Oegema, T. R., Jr., & McCarthy, J. B. (2001) Hyaluronan Synthase Elevation in Metastatic Prostate Carcinoma Cells Correlates with Hyaluronan Surface Retention, a Prerequisite for Rapid Adhesion to Bone Marrow Endothelial Cells. J. Biol. Chem., 276, 17949-17957.
[140] Knudson, W. (1996) Tumor-associated hyaluronan—Providing an extracellular matrix that facilitates invasion. Am. J. Pathol., 148, 1721-1726.
[141] McAleer, M. A., Breen, M. A., White, N. L., & Matthews, N. (1999) pABC11 (also known as MOAT-C and MRP5), a member of the ABC family of proteins, has anion transporter activity but does not confer multidrug resistance when overexpressed in human embryonic kidney 293 cells. J. Biol. Chem., 274, 23541-23548.
[142] Kool, M., De Haas, M., Scheffer, G. L., Scheper, R. J., van Eijk, M. J., Juijn, J. A., Baas, F., & Borst, P. (1997) Analysis of expression of cMOAT (MRP2), MRP3, MRP4, and MRP5, homologues of the multidrug resistance-associated protein gene (MRP1), in human cancer cell lines. Cancer Res., 57, 3537-3547.
[143] Schinkel, A. H. & Jonker, J. W. (2003) Mammalian drug efflux transporters of the ATP binding cassette (ABC) family: an overview. Adv. Drug Deliv. Rev., 55, 3-29.
[144] Lin, Z. P., Johnson, D. R., Finch, R. A., Belinsky, M. G., Kruh, G. D., & Sartorelli, A. C. (2002) Comparative study of the importance of multidrug resistance-associated protein 1 and P-glycoprotein to drug sensitivity in immortalized mouse embryonicfibroblasts. Mol. Cancer. Ther., 1, 1105-1114.
[145] Jedlitschky, G., Burchell, B., & Keppler, D. (2000) The multidrug resistance protein 5 functions as an ATP-dependent export pump for cyclic nucleotides. J. Biol. Chem., 275, 30069-30074.
[146] Wijnholds, J., Mol, C. A., van Deemter, L., De Haas, M., Scheffer, G. L., Baas, F., Beijnen, J. H., Scheper, R. J., Hatse, S., De Clercq, E., Balzarini, J., & Borst, P. (2000) Multidrug-resistance protein 5 is a multispecific organic anion transporter able to transport nucleotide analogs. Proc. Natl. Acad. Sci. U.S. A, 97, 7476-7481.
[147] Reid, G., Wielinga, P., Zelcer, N., Van, D. H., I, Kuil, A., De Haas, M., Wijnholds, J., & Borst, P. (2003) The human multidrug resistance protein MRP4 functions as a prostaglandin efflux transporter and is inhibited by nonsteroidal antiinflammatory drugs. Proc. Natl. Acad. Sci. U.S. A, 100, 9244-9249.
[148] Suzuki, T., Sasaki, H., Kuh, H. J., Agui, M., Tatsumi, Y., Tanabe, S., Terada, M., Saijo, N., & Nishio, K. (2000) Detailed structural analysis on both human MRP5 and mouse mrp5 transcripts. Gene, 242, 167-173.
[149] Flieger, O., Engling, A., Bucala, R., Lue, H., Nickel, W., & Bernhagen, J. (2003) Regulated secretion of macrophage migration inhibitory factor is mediated by a non-classical pathway involving an ABC transporter. FEBS Lett., 551, 78-86.
[150] Payen, L., Delugin, L., Courtois, A., Trinquart, Y., Guillouzo, A., & Fardel, O. (2001) The sulphonylurea glibenclamide inhibits multidrug resistance protein (MRP1) activity in human lung cancer cells. Br. J. Pharmacol., 132, 778-784.
[151] Golstein, P. E., Boom, A., van Geffel, J., Jacobs, P., Masereel, B., & Beauwens, R. (1999) P-glycoprotein inhibition by glibenclamide and related compounds. Pflugers Arch., 437, 652-660.
[152] Klokbuzas, A., Wu, C. P., van Veen, H. W., Barrand, M. A., & Hladky, S. B. (2003) cGMP and glutathione-conjugate transport in human erythrocytes. Eur. J. Biochem., 270, 3696-3708.
[153] Hollo, Z., Homolya, L., Hegedus, T., & Sarkadi, B. (1996) Transport properties of the multidrug resistance-associated protein (MRP) in human tumour cells. FEBS Lett., 383, 99-104.
[154] Bobrowska-Hagerstrand, M., Wrobel, A., Rychlik, B., Bartosz, G., Soderstrom, T., Shirataki, Y., Motohashi, N., Molnar, J., Michalak, K., & Hagerstrand, H. (2001) Monitoring of MRP-like activity in human erythrocytes: inhibitory effect of isoflavones. Blood Cells Mol. Dis., 27, 894-900.
[155] Chen, Z. S., Lee, K., Walther, S., Raftogianis, R. B., Kuwano, M., Zeng, H., & Kruh, G. D. (2002) Analysis of methotrexate and folate transport by multidrug resistance protein 4 (ABCC4): MRP4 is a component of the methotrexate efflux system. Cancer Res., 62, 3144-3150.
[156] Reid, G., Wielinga, P., Zelcer, N., De Haas, M., van Deemter, L., Wijnholds, J., Balzarini, J., & Borst, P. (2003) Characterization of the transport of nucleoside analog drugs by the human multidrug resistance proteins MRP4 and MRP5. Mol. Pharmacol., 63, 1094-1103.
[157] Buxbaum, E. (1999) Co-operative binding sites for transported substrates in the multiple drug resistance transporter Mdr1. Eur. J. Biochem., 265, 64-70.
[158] Robey, R., Bakke, S., Stein, W., Meadows, B., Litman, T., Patil, S., Smith, T., Fojo, T., & Bates, S. (1999) Efflux of rhodamine from CD56+ cells as a surrogate marker for reversal of P-glycoprotein-mediated drug efflux by PSC 833. Blood, 93, 306-314.
[159] Leier, I., Jedlitschky, G., Buchholz, U., Cole, S. P., Deeley, R. G., & Keppler, D. (1994) The MRP gene encodes an ATP-dependent export pump for leukotriene C4 and structurally related conjugates. J. Biol. Chem., 269, 27807-27810.
[160] Chen, Z. S., Kawabe, T., Ono, M., Aoki, S., Sumizawa, T., Furukawa, T., Uchiumi, T., Wada, M., Kuwano, M., & Akiyama, S. I. (1999) Effect of multidrug resistance-reversing agents on transporting activity of human canalicular multispecific organic anion transporter. Mol. Pharmacol., 56, 1219-1228.
[161] Sundkvist, E., Jaeger, R., & Sager, G. (2000) Leukotriene C(4) (LTC(4)) does not share a cellular efflux mechanism with cGMP: characterisation of cGMP transport by uptake to inside-out vesicles from human erythrocytes. Biochim. Biophys. Acta, 1463, 121-130.
[162] Cool, R. H., Veenstra, M. K., van Klompenburg, W., Heyne, R. I., Muller, M., de Vries, E. G., van Veen, H. W., & Konings, W. N. (2002) S-decyl-glutathione nonspecifically stimulates the ATPase activity of the nucleotide-binding domains of the human multidrug resistance-associated protein, MRP1 (ABCC1). Eur. J. Biochem., 269, 3470-3478.
[163] Luo, F. R., Paranjpe, P. V., Guo, A., Rubin, E., & Sinko, P. (2002) Intestinal transport of irinotecan in Caco-2 cells and MDCK II cells overexpressing efflux transporters Pgp, cMOAT, and MRP1. Drug Metab Dispos., 30, 763-770.
[164] Dick, G. M., Kong, I. D., & Sanders, K. M. (1999) Effects of anion channel antagonists in canine colonic myocytes: comparative pharmacology of Cl−, Ca2+ and K+ currents. Br. J. Pharmacol., 127, 1819-1831.
[165] Assaraf, Y. G., Rothem, L., Hooijberg, J. H., Stark, M., Ifergan, I., Kathmann, I., Dijkmans, B. A., Peters, G. J., & Jansen, G. (2003) Loss of multidrug resistance protein 1 expression and folate efflux activity results in a highly concentrative folate transport in human leukemia cells. J. Biol. Chem., 278, 6680-6686.
[166] Stark, M., Rothem, L., Jansen, G., Scheffer, G. L., Goldman, I. D., & Assaraf, Y. G. (2003) Antifolate resistance associated with loss of MRP1 expression and function in Chinese hamster ovary cells with markedly impaired export of folate and cholate. Mol. Pharmacol., 64, 220-227.
[167] Meyer, F. A., Yaron, I., Mashiah, V., & Yaron, M. (1993) Methotrexate inhibits proliferation but not interleukin 1 stimulated secretory activities of cultured human synovial fibroblasts. J. Rheumatol., 20, 238-242.
[168] Bignami, A. & Asher, R. (1992) Some observations on the localization of hyaluronic acid in adult, newborn and embryonal rat brain. Int J. Dev. Neurosci., 10, 45-57.
[169] Prehm, P. (1980) Induction of hyaluronic acid synthesis in teratocarcinoma stem cells by retinoic acid. FEBS Lett., 111, 295-298.
[170] Brecht, M., Mayer, U., Schlosser, E., & Prehm, P. (1986) Increased hyaluronate synthesis is required for fibroblast detachment and mitosis. Biochem. J., 239, 445-450.
[171] August, E. M., Nguyen, T., Malinowski, N. M., & Cysyk, R. L. (1994) Non-steroidal anti-inflammatory drugs and tumor progression: Inhibition of fibroblast hyaluronic acid production by indomethacin and mefenamic acid. Cancer Lett., 82, 49-54.

1. A method of treating a disease which is associated with an excess transport of hyaluronan across a lipid bilayer, comprising:
administering a pharmaceutical composition comprising at least one inhibitor of at least one ABC transporter capable of transporting hyaluronan across a lipid bilayer.

2. The method of claim 1, wherein said inhibitor specifically reduces the transport of hyaluronan across a lipid bilayer mediated by at least one of said ABC-transporter.

3. The method of claim 1, wherein said ABC-transporter is a mammalian ABC-transporter.

4. The method of claim 1, wherein said ABC-transporter is a human ABC-transporter.

5. The method of claim 1, wherein said human ABC-transporter is a member of the subfamily selected from the group consisting of the human ABCB (MDR)-subfamily, the ABCA subfamily and the human ABC-C (MRP)-subfamily.

6. The method of claim 1, wherein said ABC-transporter(s) is comprised in a chondrocyte cell.

7. The method of claim 1, wherein said inhibitor is selected from the group consisting of:
(a) an inhibitor of a member of the ABCB (MDR)-subfamily selected from Verapamil, Valspodar (PSC833), Elacridar (GF-120918), Bericodar (VX-710), Tariquidar (XR-9576), XR-9051, S-9788, LY-335979, MS 209, R101933; OC-144-093; Quinidine, Chloripramine, Nicardipine, Nifedipine, Amlodipine, Felodipine, Manidipine, Flunarizine, Nimodipine, Pimozide, Lomerizine, Bepridil, Amiloride, Almitrine, Amiodarone, Imipramine, Clomiphene, Tamoxifen, Toremifene, Ketocanazole, Terfenadine, Chloroquine, Mepacrin, Diltiazem, Niguldipine, Prenylamine, Gallopamil, Tiapamil, Dex-Verapamil, Dipyridamole, Pimozide, Haloperidol, Chlorpromazine, Trifluoperazine, Fluphenazine, Reserpin, Clopenthixol, Flupentixol, N-acetyldaunorubicin, Vindoline, N2762-14, N276-14, N276-17, B9309-068, BIBW-22, Carvedilol, Clofazimine, Ketoconazole, Lovastatin, N-Norgallopamil, Simvastatin, Troleandomycin, Vinblastin, Itraconazole, Econazole, Oligomycine, Cyclosporin and Rapamycin; and
(b) an inhibitor of a member of the ABCA subfamily selected from Glyburide, DIDS (4,4-diisothiocyanatostilbene-2,2-disulfonic acid), Bumetanide, Furosemide, Sulfobromophthalein, Diphenylamine-2-carboxylic acid and Flufenamic acid; and
(c) an inhibitor of a member of the human ABC-C (MRP)-subfamily selected from MK-571, Benzbromaron, PAK-104P, Probenecid, Sulfinpyrazone, Indomethacin, Merthiolate and Ethacrynic acid; and
(d) an antibody or functional fragments thereof which is specifically recognizing one or more ABC-transporter capable of transporting hyaluronan across a lipid bilayer; and
(e) an antisense oligomere, iRNA and/or siRNA directed against one or more ABC-transporter capable of transporting hyaluronan across a lipid bilayer; and
(f) an aptamer directed against one or more ABC-transporter capable of transporting hyaluronan across a lipid bilayer.

8. The method of claim 1, wherein said disease which is associated with an excess transport of hyaluronan across a lipid bilayer is arthritis.

9. The method of claim 8, wherein said arthritis is characterized by at least one of a degeneration and a destruction of cartilage.

10. The method of claim 8, wherein said arthritis is selected from the grout consisting of osteoarthritis, (juvenile) chronic arthritis, rheumatoid arthritis, psoriatic arthritis, A. mutilans, septic arthritis, infectious arthritis and reactive arthritis.

11. The method of claim 1, wherein said inhibitor is administered prophylactically.

12. The method of claim 1, wherein said inhibitor is to be administered therapeutically.

13. A method of screening for a compound which is suitable for the treatment of a disease which is associated with an excess transport of hyaluronan across a lipid bilayer said method comprising:
(a) contacting an isolated lipid bilayer comprising at least one ABC-transporter which is capable of transporting hyaluronan with a test compound and an indicator compound;
(b) measuring the effect of the test compound on the transport of the indicator compound across the lipid bilayer; and
(c) identifying test compounds which reduce the transport of the indicator compound.

14. A method of screening for a compound which reduces the transport of hyaluronan mediated by at least one ABC-transporter, said method comprising:
(a) contacting an isolated lipid bilayer comprising at least one ABC-transporter which is capable of transporting hyaluronan with a test compound and an indicator compound;
(b) measuring the effect of the test compound on the transport of the indicator compound across the lipid bilayer; and
(c) identifying test compounds which reduce the transport of the indicator compound.

15. A method of screening for a compound which is suitable for the treatment of a disease which is associated with an excess transport of hyaluronan across a lipid bilayer said method comprising:
(a) contacting a cell comprising at least one ABC-transporter which is capable of transporting hyaluronan with a test compound and an indicator compound;
(b) measuring the effect of the test compound on the transport of the indicator compound across a lipid bilayer of the cell; and
(c) identifying compounds which reduce the transport of the indicator compound.

16. A method of screening for a compound which reduces the transport of hyaluronan mediated by at least one ABC-transporter, said method comprising:
(a) contacting a cell comprising at least one ABC-transporter which is capable of transporting hyaluronan with a test compound and an indicator compound;
(b) measuring the effect of the test compound on the transport of the indicator compound across a lipid bilayer of the cell; and
(c) identifying compounds which reduce the transport of the indicator compound.

17. The method of claim 13 comprising screening for a compound which specifically reduces the transport of hyaluronan mediated by said ABC-transporter.

18. The method of claim 15, wherein the cell is a bacterial, an insect, a fungal or an animal cell.

19. The method of claim 18, wherein said animal cell is a mammalian cell or a mammalian cell line.

20. The method of claim 19, wherein said mammalian cell or mammalian cell line is derived from human, horse, swine, goat, cattle, mouse or rat.

21. The method of claim 19, wherein the cell or cell line is a chondrocyte, a fibroblast, a synovial cell, an endothelial cell, a macrophage, a tumour cell, a smooth muscle cell, a melanoma cell or a mesothelioma cell.

22. The method of claim 21, wherein said cell is comprised in a tissue.

23. The method of claim 22, wherein said tissue is cartilage tissue.

24. The method of claim 19, wherein said cell or said tissue is derived from a mammalian subject preferably a human subject which suffers from a disease which is associated with an excess transport of hyaluronan across a lipid bilayer.

25. The method of claim 19, wherein the cell comprises at least one heterologous ABC-transporter.

26. The method of claim 19, wherein said cell and/or said tissue is comprised in a non-human animal.

27. The method of claim 15 which is ex vivo.

28. A method of screening for a compound which is suitable for the treatment of a disease which is associated with an excess transport of hyaluronan across a lipid bilayer said method comprising:
(a) contacting a cell derived from said subject which comprises at least one ABC-transporter with a test compound to be tested;
(b) measuring the effect of the test compound on the transport of an indicator compound across a lipid bilayer of said cell; and
(c) identifying compounds which reduce the transport of hyaluronan across the lipid bilayer of said cell.

29. The method of claim 28, wherein said cell is comprised in a tissue.

30. The method of claim 28, wherein said cell is a chondrocyte.

31. The method of claim 28, wherein said subject is a mammalian subject.

32. The method of claim 31, wherein said mammalian subject is selected from a human, a horse, a camel, a dog, a cat, a pig, a cow and a goat.

33. The method of any one of claims 28, wherein said cell is contacted with a compound selected from the group consisting of:
(a) an inhibitor of a member of the ABCB (MDR)-subfamily selected from Verapamil, Valspodar (PSC833), Elacridar (GF-120918), Bericodar (VX-710), Tariquidar (XR-9576), XR-9051, S-9788, LY-335979, MS 209, R101933; OC-144-093; Quinidine, Chloripramine, Nicardipine, Nifedipine, Amlodipine, Felodipine, Manidipine, Flunarizine, Nimodipine, Pimozide, Lomerizine, Bepridil, Amiloride, Almitrine, Amiodarone, Imipramine, Clomiphene, Tamoxifen, Toremifene, Ketocanazole, Terfenadine, Chloroquine, Mepacrin, Diltiazem, Niguldipine, Prenylamine, Gallopamil, Tiapamil, Dex-Verapamil, Dipyridamole, Pimozide, Haloperidol, Chlorpromazine, Trifluoperazine, Fluphenazine, Reserpin, Clopenthixol, Flupentixol, N-acetyldaunorubicin, Vindoline, N2762-14, N276-14, N276-17, B9309-068, BIBW-22, Carvedilol, Clofazimine, Ketoconazole, Lovastatin, N-Norgallopamil, Simvastatin, Troleandomycin, Vinblastin, Itraconazole, Econazole, Oligomycine, Cyclosporin and Rapamycin; and
(b) an inhibitor of a member of the ABCA subfamily selected from Glyburide, DIDS (4,4-diisothiocyanatostilbene-2,2-disulfonic acid), Bumetanide, Furosemide, Sulfobromophthalein, Diphenylamine-2-carboxylic acid and Flufenamic acid; and
(c) an inhibitor of a member of the human ABC-C (MRP)-subfamily selected from MK-571, Benzbromaron, PAK-104P, Probenecid, Sulfinpyrazone, Indomethacin, Merthiolate and Ethacrynic acid; and
(d) (an) antibody or functional fragments thereof which is specifically recognizing one or more ABC-transporter capable of transporting hyaluronan across a lipid bilayer; and
(e) an antisense oligomere, iRNA and/or siRNA directed against one or more ABC-transporter capable of transporting hyaluronan across a lipid bilayer; and
(f) an aptamer directed against one or more ABC-transporter capable of transporting hyaluronan across a lipid bilayer.

34. The method of claim 13 further comprising a step of refining the compound identified, said method comprising the steps of:
(a) identification of the binding sites of the compound and the ABC-transporter;
(b) molecular modelling of the binding site of the compound; and
(c) modification of the compound to improve its binding specificity for the ABC-transporter.

35. The method of claim 13, further comprising the step of formulating the compound identified, refined or modified with at least one of a pharmaceutically active carrier and/w a diluent.

36. A method for manufacturing a pharmaceutical composition comprising the steps of claim 13 and the step of formulating the compound screened in a pharmaceutically acceptable form.

37. A method of preventing, ameliorating and/or treating the symptoms of a disease which is associated with an excess transport of hyaluronan across a lipid bilayer, e.g. arthritis in a subject comprising administering at least one inhibitor of at least one ABC-transporter capable of transporting hyaluronan across a lipid bilayer to the subject, preferably an mammalian subject, such that the a disease which is associated with an excess transport of hyaluronan across a lipid bilayer.

38. The method of claim 37, wherein said arthritis is characterized by at least one of degeneration and a destruction of cartilage.

39. The method of claim 37, wherein said arthritis is selected from the group consisting of osteoarthritis, (juvenile) chronic arthritis, rheumatoid arthritis, psoriatic arthritis, A. mutilans, septic arthritis, infectious arthritis and reactive arthritis.

40. The method of claim 39 wherein said arthritis is osteoarthritis.

41. The method of claim 37, wherein said mammalian subject is selected from the group consisting of a human, a horse, a camel, a dog, a cat, a pig, a cow and a goat.

42. The method of claim 1, wherein said ABC-transporter is selected from the group consisting of MRP5 (ABCC5), ABCC11 and ABCC12.

43. A method of treating osteoarthritis comprising administering a pharmaceutical composition comprising at least one ABC-transporter capable of transporting hyaluronan across a lipid-bilayer, wherein said at least one ABC-transporter is selected from the group consisting of MRP5 (ABCC5), ABCC11 and ABCC12.

44. The method of claim 43, wherein said at least one ABC-transporter is MRP5 (ABCC5).

45. A method of treating arthritis comprising administering a Pharmaceutical composition comprising Zaprinast®.

46. A method of treating osteoarthritis comprising administering a pharmaceutical composition comprising Elacridar (GF-120918), Valspodar (PSC-833), Bericodar (VX-710), Tariquidar (XR-9576), S-9788, Ly-335979, OC-144-093 or Lysodren®.

47. The method of claim 14 comprising screening for a compound which specifically reduces the transport of hyaluronan mediated by said ABC-transporter.

48. The method of claim 15 comprising screening for a compound which specifically reduces the transport of hyaluronan mediated by said ABC-transporter.

49. The method of claim 16 comprising screening for a compound which specifically reduces the transport of hyaluronan mediated by said ABC-transporter.

50. The method of claim 16, wherein the cell is a bacterial, an insect, a fungal or an animal cell.

51. The method of claim 14, further comprising the step of formulating the compound identified, refined or modified with at least one of a pharmaceutically active carrier and a diluent.

52. The method of claim 15, further comprising the step of formulating the compound identified, refined or modified with at least one of a pharmaceutically active carrier and a diluent.

53. The method of claim 16, further comprising the step of formulating the compound identified, refined or modified with at least one of a pharmaceutically active carrier and a diluent.

54. The method of claim 45 wherein said arthritis is selected from rheumatoid arthritis and osteoarthritis.

55. The method of claim 16 which is ex vivo.

56. The method of claim 13, wherein said ABC-transporter is selected from the group consisting of MRP5 (ABCC5), ABCC11 and ABCC12.

57. The method of claim 14, wherein said ABC-transporter is selected from the group consisting of MRP5 (ABCC5), ABCC11 and ABCC12.

58. The method of claim 15, wherein said ABC-transporter is selected from the group consisting of MRP5 (ABCC5), ABCC11 and ABCC12.

59. The method of claim 16, wherein said ABC-transporter is selected from the group consisting of MRP5 (ABCC5), ABCC11 and ABCC12.

60. The method of claim 28, wherein said ABC-transporter is selected from the group consisting of MRP5 (ABCC5), ABCC11 and ABCC12.

61. The method of claim 37, wherein said ABC-transporter is selected from the group consisting of MRP5 (ABCC5), ABCC11 and ABCC12.

62. The method of claim 14 further comprising a step of refining the compound identified, said method comprising the steps of:
(a) identification of the binding sites of the compound and the ABC-transporter;
(b) molecular modelling of the binding site of the compound; and
(c) modification of the compound to improve its binding specificity for the ABC-transporter.

63. The method of claim 15 further comprising a step of refining the compound identified, said method comprising the steps of:
(a) identification of the binding sites of the compound and the ABC-transporter;
(b) molecular modelling of the binding site of the compound; and
(c) modification of the compound to improve its binding specificity for the ABC-transporter.

64. The method of claim 16 further comprising a step of refining the compound identified, said method comprising the steps of:
(a) identification of the binding sites of the compound and the ABC-transporter;
(b) molecular modelling of the binding site of the compound; and
(c) modification of the compound to improve its binding specificity for the ABC-transporter.

65. The method of claim 128 further comprising a step of refining the compound identified, said method comprising the steps of:
(a) identification of the binding sites of the compound and the ABC-transporter;
(b) molecular modelling of the binding site of the compound; and
(c) modification of the compound to improve its binding specificity for the ABC-transporter.

66. A method for manufacturing a pharmaceutical composition comprising the steps of claims 14 and the step of formulating the compound screened in a pharmaceutically acceptable form.

67. A method for manufacturing a pharmaceutical composition comprising the steps of claims 15 and the step of formulating the compound screened in a pharmaceutically acceptable form.

68. A method for manufacturing a pharmaceutical composition comprising the steps of claims 16 and the step of formulating the compound screened in a pharmaceutically acceptable form.

69. A method for manufacturing a pharmaceutical composition comprising the steps of claims 28 and the step of formulating the compound screened in a pharmaceutically acceptable form.


SEQUENCE LISTING (8 sequences)

SEQ ID NO: 1 - DNA, 24 bp, Artificial Sequence
  primer
atgactttttcgtggctttcttct

SEQ ID NO: 2 - DNA, 23 bp, Artificial Sequence
  primer
gctttgacccaggcatacatttt

SEQ ID NO: 3 - DNA, 24 bp, Artificial Sequence
  primer
gacttgggcattgaattaccgaac

SEQ ID NO: 4 - DNA, 24 bp, Artificial Sequence
  primer
gtgaggactggctggtttgttctt

SEQ ID NO: 5 - DNA, 24 bp, Artificial Sequence
  primer
gggaaagtaccatcatggcttgag

SEQ ID NO: 6 - DNA, 24 bp, Artificial Sequence
  primer
gaaaatgctttgacccaggcatac

SEQ ID NO: 7 - DNA, 24 bp, Artificial Sequence
  primer
gccctgctgcgccactgtaagatt

SEQ ID NO: 8 - DNA, 26 bp, Artificial Sequence
  primer
tccggaactgctgtgcgaaagataaa